SEQUENCE LISTING (12 sequences)

SEQ ID NO: 1 - DNA, 21 bp, Artificial Sequence
  ORF1ab-F3
tccagatgaggatgaagaaga

SEQ ID NO: 2 - DNA, 21 bp, Artificial Sequence
  ORF1ab-B3
agtctgaacaactggtgtaag

SEQ ID NO: 3 - DNA, 41 bp, Artificial Sequence
  ORF1ab-FIP
agagcagcagaagtggcacaggtgattgtgaagaagaagag

SEQ ID NO: 4 - DNA, 40 bp, Artificial Sequence
  ORF1ab-BIP
tcaacctgaagaagagcaagaactgattgtcctcactgcc

SEQ ID NO: 5 - DNA, 22 bp, Artificial Sequence
  ORF1ab-LF
ctcatattgagttgatggctca

SEQ ID NO: 6 - DNA, 22 bp, Artificial Sequence
  ORF1ab-LB
acaaactgttggtcaacaagac

SEQ ID NO: 7 - DNA, 19 bp, Artificial Sequence
  GeneN-A-F3
tggctactaccgaagagct

SEQ ID NO: 8 - DNA, 20 bp, Artificial Sequence
  GeneN-A-B3
tgcagcattgttagcaggat

SEQ ID NO: 9 - DNA, 41 bp, Artificial Sequence
  GeneN-A-FIP
tctggcccagttcctaggtagtccagacgaattcgtggtgg

SEQ ID NO: 10 - DNA, 40 bp, Artificial Sequence
  GeneN-A-BIP
agacggcatcatatgggttgcacgggtgccaatgtgatct

SEQ ID NO: 11 - DNA, 25 bp, Artificial Sequence
  GeneN-A-LF
ggactgagatctttcattttaccgt

SEQ ID NO: 12 - DNA, 21 bp, Artificial Sequence
  GeneN-A-LB
actgagggagccttgaataca

(19) United States
(12) Patent Application Publication
(10) Pub. No.: US 2023/0234050 A1
(43) Pub. Date: Jul. 27, 2023

(54) MICROFLUIDIC CHIP AND SYSTEM

(71) Applicant: National University of Singapore, Singapore (SG)

(72) Inventors: Chwee Teck LIM, Singapore (SG); Quoc Mai Phuong NGUYEN, Singapore (SG); Yongling Adelicia LI, Singapore (SG); Ming WANG, Singapore (SG); Nishanth VENUGOPAL MENON, Singapore (SG); Minru Gordon XIONG, Singapore (SG); Ali Asgar SALEEM BHAGAT, Singapore (SG); Jee Chin TEOH, Singapore (SG)

(21) Appl. No.: 18/000,053

(22) PCT Filed: May 27, 2021
(86) PCT No.: PCT/SG2021/050294

§ 371 Date: Nov. 28, 2022

(30) Foreign Application Priority Data
May 28, 2020 (SG) 10202005023R
Aug. 31, 2020 (SG) 10202008413X

(51) Int. Cl. B01L 3/00

(52) U.S. Cl. CPC B01L 3/502715; B01L 2300/0816; B01L 2300/18; B01L 2300/0861; B01L 2200/0689

(57) ABSTRACT

A microfluidic chip is disclosed herein. In a specific embodiment, the microfluidic chip comprises at least one microfluidic reservoir having a wall portion and a heat transfer sealing layer cooperating with the wall portion for receiving a sample to be tested. The heat transfer sealing layer is arranged to be contiguous with the sample to be tested. The microfluidic chip further comprises an active temperature control device arranged to provide structural support to the heat transfer sealing layer and operable to control a temperature of the sample via transmission of heat through the heat transfer sealing layer. A detection module is also disclosed.

### CROSS REFERENCE TO RELATED APPLICATION(S)

The present application is a filing under 35 U.S.C. 371 as the National Stage of International Application No. PCT/SG2021/050294, filed May 27, 2021, entitled “MICROFLUIDIC CHIP AND SYSTEM,” which claims priority to Singapore Application No. SG 10202008413X filed with the Intellectual Property Office of Singapore on Aug. 31, 2020 and claims priority to Singapore Application No. SG 10202005023R filed with the Intellectual Property Office of Singapore on May 28, 2020, all of which are incorporated herein by reference in their entirety for all purposes.

### INCORPORATION BY REFERENCE OF MATERIAL IN ASCII TEXT FILE

This application incorporates by reference the Sequence Listing contained in the following ASCII text file being submitted concurrently herewith:

File name: 4373-18100_SP102702USZSO_SP102702WO_ST25; created on Nov. 18, 2022; and having a file size of 3 KB.

The information in the Sequence Listing is incorporated herein in its entirety for all purposes.

### TECHNICAL FIELD

This invention relates to a microfluidic chip and system, particularly the thermal monitoring and control of a sample/specimen on a microfluidic chip.

### BACKGROUND

Effective containment of infectious disease requires rapid and accurate detection of the relevant pathogens. The SARS-Cov-2 genome was published on 24 Jan. 2020 (Zhu et al. (2020), A Novel Coronavirus from Patients with Pneumonia in China, NEJM). This enabled laboratories around the world to develop rapid detection of the Covid-19 virus. However, most tests are carried out in laboratories with multiple specialized equipment. This delays detection and hinders containment efforts. Point of care diagnosis is therefore preferable.

Ideally, diagnostic systems enable temperature control of the samples (as may be required according to the diagnostic protocol employed) and are capable of detecting multiple samples simultaneously, enabling rapid throughput.

Microfluidics concerns the behaviour of tiny quantity of liquids on the μl scale, typically contained within small channels on so-called microfluidic chips. The use of microfluidic samples for diagnostic testing, particularly point of care diagnostic testing is advantageous because it enables testing using minute amounts of sample, potentially enabling quicker detection and deployment with less reagent.

However, existing microfluidic chips are typically made of glass and/or PDMS and/or plastic. These materials have very low thermal conductivity and are unsuitable for applications requiring fast and precise temperature control within the microfluidic channel.

Existing solutions to this problem include using electrical energy to heat fluids within microfluidic channels, i.e. electric current is applied through the fluids themselves. (e.g. Joule heating, or resistive heating on PDMS chip.) However, not all samples can be electrically heated because the electrical properties might affect the flow or because an additional solute needs to be added to alter the electrical properties of the reaction medium, giving rise to unintended side-effects. Furthermore, the sample/specimen/process may respond to the external electric field that is being applied.

Point of care diagnosis often requires a detection system that is portable and provides a quick read-out. Portable fluorescence detectors known in the art typically contain a light source, optical filters, focusing lens, and one or an array of photodiode(s). The excitation light is generally focused at a point (point source) on a sample to obtain a clear fluorescence signal. Existing systems are typically bulky or inefficient.

There is a continuing need to develop a cost effective, portable and highly sensitive detector systems.

### SUMMARY

In a first aspect, there is provided a microfluidic chip. The microfluidic chip comprises: at least one microfluidic reservoir having a wall portion and a heat transfer sealing layer cooperating with the wall portion for receiving a sample to be tested, the heat transfer sealing layer being arranged to be contiguous with the sample to be tested; and an active temperature control device arranged to provide structural support to the heat transfer sealing layer and operable to control a temperature of the sample via transmission of heat through the heat transfer sealing layer.

By having a heat transfer sealing layer structurally supported by an active temperature control device and cooperating with a wall portion of the microfluidic reservoir, precise control of the temperature of the microfluidic reservoir may be achieved using a device which has a simple design and is straightforward to manufacture using existing techniques for producing microfluidic chips. Indeed, with the active temperature control device, this means that the temperature control device is a heat generation source or the source for increasing or reducing temperature.

As used herein, a microfluidic reservoir, is a reservoir for receiving fluid, the reservoir having at least one dimension in the range 0.1 μm to 2 mm, for example a depth in the range 0.1 μm to 2 mm.

The transmission of heat through the heat transfer sealing layer may include transmission of heat from the sample to the active temperature control device. Additionally, or alternatively, the transmission of heat through the heat transfer sealing layer may include transmission of heat to the sample from the temperature control device, i.e. the temperature control device may cause heating or cooling of a sample received in the at least one microfluidic reservoir.

The heat transfer sealing layer may include an adhesive layer. The heat transfer sealing layer may be integral with the active temperature control device. The active temperature control device may be a thermoelectric heat pump (i.e. a solid-state thermoelectric device, or equivalently Peltier device) or a fluidic heat exchanger. The heat transfer sealing layer may comprise a metallic film.

The wall portion may define a first microfluidic reservoir profile and wherein the microfluidic reservoir profile comprises a first periodically oscillating section. Inclusion of an oscillating section the microfluidic reservoir profile may prevent a sample from leaking from the microfluidic reservoir under temperature changes. An oscillating section may further enhance mixing of the sample.

The first microfluidic reservoir profile may further comprise two or more substantially linear sections and a junction fluidically connecting the two or more substantially linear sections to the first periodically oscillating section.

The wall portion may define a second microfluidic reservoir profile and the second microfluidic reservoir profile may comprise a first periodically oscillating section; a second periodically oscillating section; a third section arranged between the first and second periodically oscillating sections, the third section having a non-oscillating configuration and comprising a first chamber region; first and second tapering portions which fluidically connect the first and second periodically oscillating sections, respectively, to the first chamber region; and a width, the width being greater at the first chamber region than in the first and second oscillating sections. The first and second tapering portions may be curved. The curved tapering portions may prevent a sample received in the reservoir from breaking up into smaller portions following temperature cycling. Alternatively, the first and second tapering portions is substantially straight. Straight tapering portions may enhance ease of manufacturing.

The third section may further comprise a second chamber region, and the microfluidic reservoir profile may further comprise third and fourth tapering portions which fluidically connect the first and second periodically oscillating sections to the second chamber region, respectively, wherein the width is greater at the second chamber region than in the first and second periodically oscillating sections.

The wall portion may define a fourth microfluidic reservoir profile and the microfluidic chip may further comprise an elastomeric seal arranged to surround the fourth microfluidic reservoir profile. The heat transfer sealing layer may cooperate with the wall portion via the elastomeric seal.

The at least one microfluidic reservoir may further comprise at least one of an inlet and an outlet operable to receive a plug. The microfluidic chip may further comprise the plug.

The at least one microfluidic reservoir may comprise at least one of an inlet and an outlet, the microfluidic chip further comprising an adhesive sealing layer arranged to seal the at least one of the inlet and the outlet.

The microfluidic chip may further include a reservoir plate comprising the wall portion and an edge surface, and wherein the at least one microfluidic reservoir comprises a third inlet comprising an aperture in the edge surface of the reservoir plate. The at least one microfluidic reservoir may comprise a microcuvette.

The microfluidic chip may comprise a plurality of the microfluidic reservoirs, each microfluidic reservoir having a respective wall portion, wherein the heat transfer sealing layer cooperates with each of the respective wall portions for receiving a respective sample to be tested, the heat transfer sealing layer being arranged to be contiguous with each respective sample to be tested, the active temperature control device operable to control a temperature of each of the respective samples via transmission of heat through the heat transfer sealing layer. Each respective wall portion may comprise a through hole. Each respective wall portion may define a respective microfluidic reservoir profile, and at least two of the respective reservoir profiles may differ in at least one of shape and sample capacity.

In a second aspect, there is provided a microfluidic chip comprising at least one microfluidic reservoir having a wall portion and a heat transfer sealing layer cooperating with the wall portion for receiving a sample to be tested, the heat transfer sealing layer being arranged to be contiguous with the sample to be tested. The microfluidic chip further comprises a fluidic heat exchanger operable to control a temperature of the sample via transmission of heat through the heat transfer sealing layer. The heat transfer sealing layer may be a metal plate.

In a third aspect, there is provided a microfluidic system comprising a microfluidic chip. The microfluidic chip includes at least one microfluidic reservoir having a wall portion and a heat transfer sealing layer cooperating with the wall portion for receiving a sample to be tested, the heat transfer sealing layer being arranged to be contiguous with the sample to be tested; and an active temperature control device arranged to provide structural support to the heat transfer sealing layer and operable to control a temperature of the sample via transmission of heat through the heat transfer sealing layer. The microfluidic system further comprises a further wall portion operable to replace the wall portion to cooperate with the heat transfer sealing layer to form a further at least one microfluidic reservoir for receiving the sample to be tested

In a fourth aspect, there is provided a microfluidic platform comprising a plurality of fluidically connected microfluidic chips, the plurality of microfluidic chips comprising one or more microfluidic chips comprising: at least one microfluidic reservoir having a wall portion and a heat transfer sealing layer cooperating with the wall portion for receiving a sample to be tested, the heat transfer sealing layer being arranged to be contiguous with the sample to be tested; and an active temperature control device arranged to provide structural support to the heat transfer sealing layer and operable to control a temperature of the sample via transmission of heat through the heat transfer sealing layer. The plurality of microfluidic chips may additionally comprise one or more microfluidic chips comprising at least one microfluidic reservoir having a wall portion and a heat transfer sealing layer cooperating with the wall portion for receiving a sample to be tested, the heat transfer sealing layer being arranged to be contiguous with the sample to be tested; and a fluidic heat exchanger operable to control a temperature of the sample via transmission of heat through the heat transfer sealing layer.

In a fifth aspect, there is provided a detection module for detecting optical signals from a plurality of samples. The detection module comprises: an ambient light shielding cassette; and a slot configured to receive the samples; the ambient light shielding cassette comprising: a first light source operable to illuminate the plurality of samples to produce respective optical signals; and a first camera operable to receive the respective optical signals simultaneously and directly from each of the samples.

By “directly” it is intended to mean that the optical signals are neither focused nor dispersed between the samples and the camera, i.e. that they do not pass through a lens. It does not preclude the use of a polariser or filter in the optical path. The first camera may or may not comprise a lens.

Each of the samples may be received in a microfluidic reservoir of a microfluidic chip comprising a plurality of microfluidic reservoirs. The microfluidic chip may comprise at least one microfluidic reservoir having a wall portion and a heat transfer sealing layer cooperating with the wall portion for receiving a sample to be tested, the heat transfer sealing layer being arranged to be contiguous with the sample to be tested; and an active temperature control device arranged to provide structural support to the heat transfer sealing layer and operable to control a temperature of the sample via transmission of heat through the heat transfer sealing layer. The microfluidic chip may comprise at least one microfluidic reservoir having a wall portion and a heat transfer sealing layer cooperating with the wall portion for receiving a sample to be tested, the heat transfer sealing layer being arranged to be contiguous with the sample to be tested; and a fluidic heat exchanger operable to control a temperature of the sample via transmission of heat through the heat transfer sealing layer.

The detection module may comprise one or more filters and/or polarisers in an optical path between the plurality of samples and the first camera. A first filter in the optical path may be operable to be removed from the optical path and replaced with a second filter. The detection module may comprise a disk in which the first and second filters are mounted, and wherein the first filter is operable to be removed from the optical path and replaced with the second filter by the rotation of the disk. The detection module may comprise a first cartridge in which the first filter is mounted, the first cartridge being mounted within the ambient light shielding cassette; and a second cartridge in which the second filter is mounted, and the first filter may be operable to be removed from the optical path and replaced with a second filter by means of the removal of the first cartridge from the ambient light shielding cassette and replacement of the first cartridge within the ambient light shielding cassette by the second cartridge.

The detection module may further comprise a second camera operable to receive the respective optical signals simultaneously and directly from each of the samples. The detection module may further comprise a second light source, wherein the first light source and the second light source are operable to produce light with different wavelengths. The first light source may be a multi-channel light source.

The first camera and slot may be arranged such that an optical signal from four or more of the plurality of samples is received simultaneously at the first camera. A field of view of the first camera may be at least 40×40 mm².

In a sixth aspect, there is provided a method of detecting a signal from a plurality of samples, the samples being received in one of: a plurality microfluidic reservoirs of a microfluidic chip, the method comprising: illuminating the plurality of samples using a light source to produce respective optical signals; and simultaneously and directly receiving each of the respective optical signals at a camera. The samples may be received in a microfluidic chip comprising: a plurality of microfluidic reservoirs, each microfluidic reservoir having a respective wall portion, wherein a heat transfer sealing layer cooperates with each of the respective wall portions for receiving a respective sample to be tested, the heat transfer sealing layer being arranged to be contiguous with each respective sample to be tested, and an active temperature control device arranged to provide structural support to the heat transfer sealing layer and operable to control a temperature of each of the respective samples via transmission of heat through the heat transfer sealing layer. Simultaneously and directly receiving each of the respective optical signals at a camera may comprise capturing all of the respective optical signals as a single image. The method may consequently further comprise: processing the respective optical signals using a processor by dividing the single image into a plurality of cropped images, such that each of the cropped images corresponds to a single sample.

In a seventh aspect, there is provided a microfluidic platform, the microfluidic platform comprising: a microfluidic chip comprising a plurality of microfluidic reservoirs; a detection module for detecting optical signals from a plurality of samples, the detection module comprising: an ambient light shielding cassette; and a slot configured to receive the samples; the ambient light shielding cassette comprising: a first light source operable to illuminate the plurality of samples to produce respective optical signals; and a first camera operable to receive the respective optical signals simultaneously and directly from each of the samples; and a system comprising: an input for receiving an image from the detection module; a processor configured to divide the image into a plurality of cropped images, such that each of the cropped images corresponds to a single sample; and an output configured to output the plurality of cropped images. The microfluidic chip may comprise: at least one microfluidic reservoir having a wall portion and a heat transfer sealing layer cooperating with the wall portion for receiving a sample to be tested, the heat transfer sealing layer being arranged to be contiguous with the sample to be tested; and an active temperature control device arranged to provide structural support to the heat transfer sealing layer and operable to control a temperature of the sample via transmission of heat through the heat transfer sealing layer.

In an eighth aspect, there is provided a system for performing one or more microfluidic temperature-controlled processes concurrently, the system comprising a plurality of microfluidic chips each comprising: at least one microfluidic reservoir having a wall portion and a heat transfer sealing layer cooperating with the wall portion for receiving a sample to be tested, the heat transfer sealing layer being arranged to be contiguous with the sample to be tested; and an active temperature control device arranged to provide structural support to the heat transfer sealing layer and operable to control a temperature of the sample via transmission of heat through the heat transfer sealing layer. The system may further comprise a plurality of detector modules, one for each microfluidic chip, each detector module suitable for detecting optical signals from a plurality of samples, the detection module comprising: an ambient light shielding cassette; and a slot configured to receive the samples;

the ambient light shielding cassette comprising: a first light source operable to illuminate the plurality of samples to produce respective optical signals; and a first camera operable to receive the respective optical signals simultaneously and directly from each of the samples.

In a ninth aspect, there is provided a method of detecting a signal from a plurality of samples, the samples being received in one of: a plurality microfluidic reservoirs of a microfluidic chip and a paper-based platform. The method comprises: illuminating the plurality of samples using a light source to produce respective optical signals; and simultaneously and directly receiving each of the respective optical signals at a camera.

In a tenth aspect, there is provided a device, the device comprising: a microfluidic chip comprising one or more microfluidic channels; and a thermal control element, wherein at least a portion of a wall of each of the one or more microfluidic channels comprises a heat transfer sealing layer and wherein the heat transfer sealing layer is arranged to transmit heat energy between the thermal control element and the microfluidic channel. The device may be fluidically connected to one or more other microfluidic devices.

In an eleventh aspect, there is provided a method of detecting a signal from a plurality of samples the samples being received in one of: a plurality microfluidic channels of a microfluidic chip, a multiwell plate and a paper-based platform, the method comprising: illuminating the plurality of samples using a light source to produce respective optical signals; and simultaneously and directly receiving each of the respective optical signals at a camera.

In a twelfth aspect, there is provided a detection module for detecting a signal from a microfluidic device, the microfluidic device comprising: a microfluidic chip comprising a plurality of microfluidic channels; and a thermal control element, wherein at least a portion of a wall of each of the plurality of microfluidic channels comprises a heat transfer layer and wherein the heat transfer layer is arranged to transmit heat energy between the thermal control element and the microfluidic channel, the detection module comprising: an ambient light shielding cassette; and a slot configured to receive the microfluidic device, the ambient light shielding cassette housing: a light source operable to illuminate the plurality of microfluidic channels to produce respective optical signals; and a camera operable to receive the respective optical signal simultaneously and directly from each of the microfluidic channels.

In a thirteenth aspect, there is provided a method of detecting a signal from a microfluidic device, the microfluidic device comprising: a microfluidic chip comprising a plurality of microfluidic channels; and a thermal control element, wherein at least a portion of a wall of each of the plurality of microfluidic channels comprises a heat transfer layer and wherein the heat transfer layer is arranged to transmit heat energy between the thermal control element and the microfluidic channel, the method comprising: illuminating the microfluidic device using a light source to produce respective optical signals; and simultaneously and directly receiving each of the respective optical signals at a camera.

In a fourteenth aspect, there is provided a method of processing an image received by a detection module for detecting optical signals from a plurality of samples, the detection module comprising: an ambient light shielding cassette; and a slot configured to receive the samples; the ambient light shielding cassette comprising: a light source operable to illuminate the plurality of samples to produce respective optical signals; and a first camera operable to receive the respective optical signals simultaneously and directly from each of the samples, the method comprising dividing the image into a plurality of cropped images, such that each of the cropped images corresponds to a single sample.

In a fifteenth aspect, there is provided a method of processing an image received by a detection module for detecting a signal from a microfluidic device, the microfluidic device comprising: a microfluidic chip comprising a plurality of microfluidic channels; and a thermal control element, wherein at least a portion of a wall of each of the plurality of microfluidic channels comprises a heat transfer layer and wherein the heat transfer layer is arranged to transmit heat energy between the thermal control element and the microfluidic channel, the detection module comprising: an ambient light shielding cassette; and a slot configured to receive the microfluidic device, the ambient light shielding cassette housing: a light source operable to illuminate the plurality of microfluidic channels to produce respective optical signals; and a camera operable to receive the respective optical signal simultaneously and directly from each of the microfluidic channels, the method comprising: dividing the image into a plurality of cropped images, such that each of the cropped images corresponds to a single sample.

In a sixteenth aspect, there is provided a system for processing an image received by a detection module for detecting optical signals from a plurality of samples, the detection module comprising: an ambient light shielding cassette; and a slot configured to receive the samples; the ambient light shielding cassette comprising: a light source operable to illuminate the plurality of samples to produce respective optical signals; and a first camera operable to receive the respective optical signals simultaneously and directly from each of the samples, the system comprising: an input for receiving an image from the detection module; processor configured to perform a method of processing an image received by the detection module, the method comprising dividing the image into a plurality of cropped images, such that each of the cropped images corresponds to a single sample; and an output configured to output the plurality of cropped images.

In an seventeenth aspect, there is provided a microfluidic platform, the platform comprising: a detection module for detecting a signal from a microfluidic device, the microfluidic device comprising: a microfluidic chip comprising a plurality of microfluidic channels; and a thermal control element, wherein at least a portion of a wall of each of the plurality of microfluidic channels comprises a heat transfer layer and wherein the heat transfer layer is arranged to transmit heat energy between the thermal control element and the microfluidic channel, the detection module comprising: an ambient light shielding cassette; and a slot configured to receive the microfluidic device, the ambient light shielding cassette housing: a light source operable to illuminate the plurality of microfluidic channels to produce respective optical signals; and a camera operable to receive the respective optical signal simultaneously and directly from each of the microfluidic channels; and a system for processing an image received by the detection module, the system comprising: an input for receiving an image from the detection module; a processor configured to perform a method of processing an image received by the detection module, the method comprising: dividing the image into a plurality of cropped images, such that each of the cropped images corresponds to a single sample; and an output configured to output the plurality of cropped images.

In an eighteenth aspect, there is provided a system for performing one or more microfluidic temperature-controlled processes concurrently, the system comprising a plurality of microfluidic devices, each device comprising: a microfluidic chip comprising one or more microfluidic channels; and a thermal control element, wherein at least a portion of a wall of each of the one or more microfluidic channels comprises a heat transfer layer and wherein the heat transfer layer is arranged to transmit heat energy between the thermal control element and the microfluidic channel.

In a nineteenth aspect, there is provided a system for performing one or more microfluidic temperature-controlled processes concurrently, the system comprising a plurality of microfluidic devices, each device comprising: a microfluidic chip comprising one or more microfluidic channels; and a thermal control element, wherein at least a portion of a wall of each of the one or more microfluidic channels comprises a heat transfer layer and wherein the heat transfer layer is arranged to transmit heat energy between the thermal control element and the microfluidic channel, the system further comprising a plurality of detector modules, one for each microfluidic chip, each detector module comprising: a light source operable to illuminate the plurality of microfluidic channels on the respective microfluidic chip to produce respective optical signals; and a camera operable to receive the respective optical signals simultaneously and directly from each of the microfluidic channels.

In a twentieth aspect, there is provided a computer readable medium configured to cause a processor to perform a method of processing an image received by a detection module for detecting a signal from a microfluidic device, the microfluidic device comprising: a microfluidic chip comprising a plurality of microfluidic channels; and a thermal control element, wherein at least a portion of a wall of each of the plurality of microfluidic channels comprises a heat transfer layer and wherein the heat transfer layer is arranged to transmit heat energy between the thermal control element and the microfluidic channel, the detection module comprising: an ambient light shielding cassette; and a slot configured to receive the microfluidic device, the ambient light shielding cassette housing: a light source operable to illuminate the plurality of microfluidic channels to produce respective optical signals; and a camera operable to receive the respective optical signal simultaneously and directly from each of the microfluidic channels, the method comprising: dividing the image into a plurality of cropped images, such that each of the cropped images corresponds to a single sample.

The computer readable medium may be transitory or non-transitory.

It is envisaged that features relating to one aspect may be applicable to the other aspects.

### BRIEF DESCRIPTION OF FIGURES

Features and non-limiting embodiments of the present invention are described in association with the figures, in which:

FIGS. 1(a) and (b) illustrate a cross-sectional view of a microfluidic chip according to an embodiment;

FIGS. 1(c) and (d) illustrate a microfluidic chip according to another embodiment;

FIGS. 2(a) and (b) illustrate a microfluidic chip according to another embodiment;

FIG. 3 shows a microfluidic platform including a microfluidic chip according to the embodiments of FIGS. 1(a) and (b) comprising a thermoelectric heat pump;

FIG. 4 illustrates a platform having a similar configuration to that of FIG. 3;

FIG. 5 schematically illustrates the platform of FIG. 4;

FIGS. 6(a)-(c) illustrate the microfluidic chip of the platform of FIG. 4;

FIG. 6(d) illustrates an alternative channel profile for the microfluidic chip of FIGS. 6(a)-(c);

FIGS. 7(a) to (e) illustrate two alternative microfluidic chips for use with the platform of FIG. 4.

FIG. 8 shows a further alternative microfluidic chip for use with the platform of FIG. 4;

FIGS. 9(a) to (e) show microfluidic channel profiles for use with the microfluidic chips of FIGS. 7 and 8;

FIGS. 10(a)-(c) illustrate microfluidic chips according to the embodiments of FIGS. 2, 1(a) and (b) and 1(c) and (d), respectively comprising fluidic heat exchangers;

FIGS. 11(a) and (b) show channel plates for use with the microfluidic chips of FIGS. 10(a)-(c);

FIGS. 12(a)-(c) show a microfluidic chip according to the embodiment of FIG. 10(a);

FIGS. 13(a)-(c) show a microfluidic chip having a smaller heat transfer sealing layer than that of FIGS. 12(a) to (c);

FIGS. 14(a) and (b) show an assembly for use in a microfluidic chip according to the embodiment of FIG. 10(a);

FIGS. 15(a) and (b) show an assembly for use in a microfluidic chip according to the embodiment of FIG. 10(a);

FIGS. 16(a)-(f) illustrate channel plates for use in microfluidic chips according to embodiments;

FIGS. 17(a) to (f) illustrate further channel plates for use in microfluidic chips according to embodiments;

FIGS. 18(a)-(c) show microfluidic platforms according to embodiments;

FIG. 19 shows a microfluidic platform according to an embodiment;

FIGS. 20(a) and (b) show microfluidic platforms according to embodiments;

FIGS. 21(a)-(f) show detector devices according to embodiments;

FIG. 22 shows a detector according to an embodiment;

FIGS. 23(a) and (b) show LED arrays according to embodiments;

FIG. 24 shows an image processing method according to an embodiment;

FIG. 25 shows an image processing method according to an embodiment;

FIG. 26 shows a method of operating a microfluidic platform according to an embodiment;

FIG. 27 shows an example temperature profile for operating a microfluidic device;

FIG. 28 shows an example fluorescence signal and Ct determination;

FIG. 29 shows a schematic of a microfluidic platform according to an embodiment;

FIG. 30 shows a schematic of a microfluidic platform according to an embodiment;

FIG. 31 shows a schematic of a microfluidic platform according to an embodiment;

FIG. 32 shows results for temperature control performed by a device according to an embodiment;

FIG. 33 shows results for temperature control performed by a device according to an embodiment;

FIG. 34 shows results for temperature control performed by a device according to an embodiment;

FIG. 35 shows results for temperature control performed by a device according to an embodiment;

FIG. 36(a)-(c) show a process for channel loading of a device according to an embodiment;

FIG. 36(d) shows results for temperature control performed by the device of FIGS. 36(a)-(c);

FIG. 37 shows results for transformation DNA efficiency using a device according to an embodiment;

FIG. 38 shows results for detection intensity using a detector according to an embodiment;

FIGS. 39(a) and (b) show detection signals for a prior-art detector and a detector according to an embodiment, respectively;

FIGS. 40(a)-(d) show benchmarking results for nucleic acid amplification using a device according to an embodiment;

FIG. 41 shows the amplification plot during corresponding to the nucleic acid amplification of FIG. 40;

FIG. 42 shows a comparison of fluorescence intensity with different substrate materials;

FIG. 43 shows results for fluorescence detection with a detector according to an embodiment; and

FIGS. 44(a) and (b) show amplification plots determined using an image processing daemon according to an embodiment.

### DETAILED DESCRIPTION

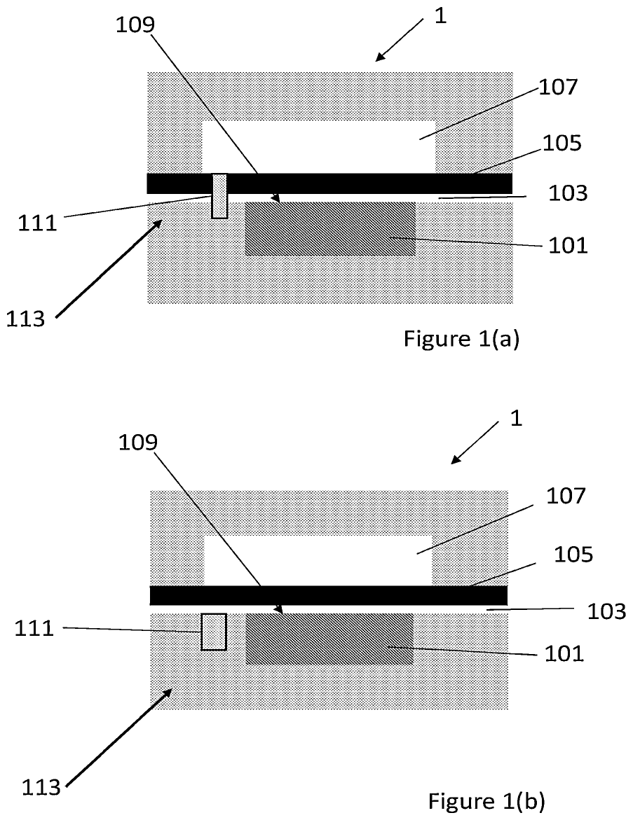

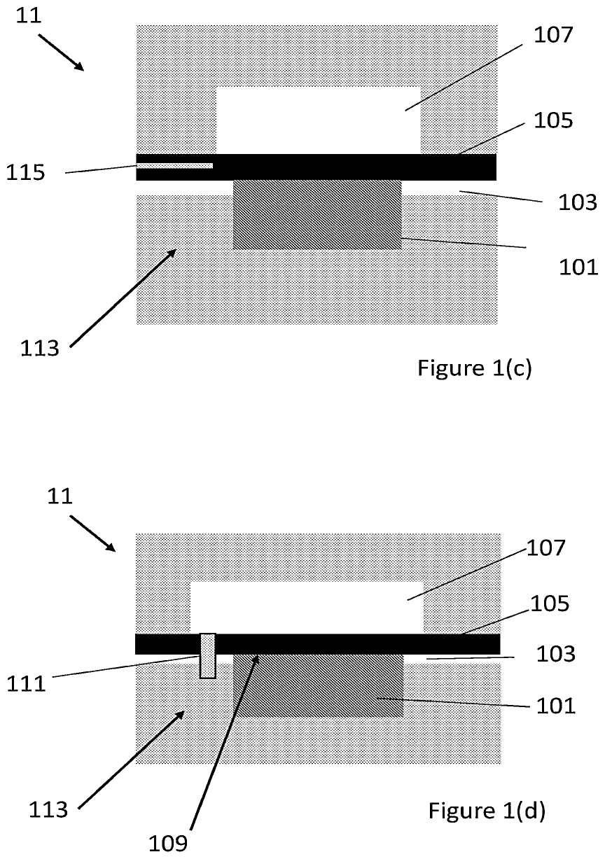

FIG. 1(a) shows a simplified schematic diagram of a microfluidic chip 1 according to an embodiment. The microfluidic chip 1 is shown in a cross-sectional profile. In this embodiment, the microfluidic chip 1 comprises a microfluidic reservoir in the form of microchannel 101. The microfluidic chip 1 comprises a main channel plate 113, equivalently reservoir plate, carrying a microchannel profile; a heat transfer sealing layer 105 including an adhesive layer 103 on one side of the heat transfer sealing layer 105 facing the main channel plate 113; and an active temperature control device, or equivalently, thermally active element 107.

The microchannel 101 includes a wall portion and the heat transfer sealing layer 105 cooperates with the wall portion to receive a sample in the microchannel 101, and thus, the heat transfer sealing layer 105 forms an enclosing member 109 for the microchannel 101. It should be appreciated that the adhesive layer 103 comprised within the heat transfer sealing layer 105 is used to adhere the heat transfer sealing layer 105 to the wall portion (as to form the microfluidic reservoir), although the adhesive layer 103 may be considered optional and other ways may be used.

The thermally active element 107 is operable to actively heat or cool the microchannel 101 via the transmission of heat through the heat transfer sealing layer 105 in order to enable temperature control of the sample in the microchannel 101. By actively heating or cooling it is intended to mean that the thermally active element 107 itself generates the temperature gradient required for heat transfer in or out of the microchannel 101, without reliance on an external device or heat source. Thus, in the embodiment of FIG. 1(a), all of the components required for temperature control of the microchannel 101 are integrated into the microfluidic chip 1. Examples of devices which may be employed as the thermally active element 107 include (but are not limited to) thermoelectric heat pumps (i.e., a solid-state thermoelectric device, or equivalently a Peltier device) and fluidic heat exchanges. Embodiments employing both types of devices will be described in detail below.

In common with the channels of the other embodiments described herein, the dimensions of the microchannel 101 are not particularly limited, however at least one dimension of the microchannel 101 is particularly in the range from about 0.1 μm to about 2 mm to ensure microfluidic behaviour of a fluid received within the microchannel 101. For example, the depth of the microchannel 101 may be in the range from about 0.1 μm to about 2 mm.

In this embodiment, the thermally active element 107 is in direct thermal contact, such as in direct physical contact or via a sandwiched layer of thermal compound or thermal paste, with the heat transfer sealing layer 105 for direct heating/cooling. In either case, the thermally active element 107 provides structural support to the heat transfer sealing layer 105, with the heat transfer sealing layer 105 being backed by the thermally active element 107, without which the heat transfer sealing layer 105 may not be able to hold the sample to be tested. In this embodiment, the heat transfer sealing layer 105 (optionally including the adhesive layer 103), which has higher thermal conductivity relative to the wall portion, is arranged to be contiguous with the sample to be tested and thereby enables both efficient and rapid heating/cooling of the sample/specimen.

In an embodiment the heat transfer sealing layer 105 may be less than about 2000 μm in thickness, excluding the optional adhesive layer 103. In another embodiment, the heat transfer sealing layer 105 (excluding the optional adhesive layer 103) may be less than about 200 μm in thickness, particularly less than about 50 μm in thickness. In yet another embodiment, the heat transfer sealing layer 105 (excluding the optional adhesive layer 103) may be about 36 μm or less. Specifically, the heat transfer sealing layer 105 (excluding the optional adhesive layer 103) may be as thin as about 0.001 μm and this may be the lower limit for all possible embodiments discussed above

In an embodiment, the heat transfer sealing layer 105 may comprise a film, i.e. a thin, flexible, or equivalently pliable sheet of material. In an embodiment, the film may be a metallic film. In an embodiment, the film may be a copper or aluminium film. Advantageously metallic films provide high thermal conductance.

In another embodiment, the film may be a plastic film. Advantageously, plastic films may provide straightforward manufacturing by enabling the use of lamination to apply the film to the main channel plate 113.

By using only a very thin piece of material in the heat transfer sealing layer 105, such as a film, the temperature control response can be fast. This enables minimal change to the profile of the microfluidic chip 1 relative to existing designs and therefore enables easy integration of the microfluidic chip 1 into existing microfluidic platforms without requiring adaptation such as special considerations for channel design and operation to increase efficiency of heat transfer. Further, as the film forms one of the walls of the micro channel 101 thereby sealing the open micro channel 101, integration of the film into the microfluidic chip 1 can be achieved easily using existing microfluidic chip manufacturing techniques such as bonding, lamination, etc. Further, providing the thermally conductive material as a film means that its size and shape can be easily adapted according to the requirements of the temperature control for the microfluidic chip 1 enabling both localised and whole-channel temperature control as well as being adaptable to any chip design. This will become evident from the discussion below. Further, by providing structural support to the film via the thermally active element 107 improved sealing of the microchannel 101 for receiving a sample may be achieved.

In other embodiments, particularly those in which the thermally active element 107 is a fluidic heat exchanger, the heat transfer sealing layer 105 may alternatively comprise a thin plate, i.e. a thin rigid sheet of material, for example a metallic plate. The thin plate may be at least 0.1 mm in thickness and comprise copper shim/plate, aluminium shim/plate. The thin plate may be formed using wire cut Electrical Discharge Machining (EDM), die cutting, or manual cutting with a scissor. Advantageously, wire cut EDM provides high accuracy.

In an embodiment the thermal conductivity of the heat transfer sealing layer 105 (excluding the optional adhesive layer 103), whether comprising a film or a plate or any other material, is greater than about 1 W/mK. In particular, it may be greater than about 100 W/mK, further particularly greater than about 200 W/mK. Advantageously, in this way, the heat transfer sealing layer 105 has a higher thermal conductivity and may enable efficient heat transfer and precise control of the temperature profile of the sample in the microchannel 101.

In an embodiment, the heat transfer sealing layer 105 comprises a metallic material. The metallic material advantageously provides high thermal conductivity thereby advantageously providing a wider range of temperature control and fast heating. For example, copper has a thermal conductivity of about 385-400 W/mK, aluminium has a thermal conductivity of about 200-237 W/mK. This is a few orders of magnitude higher than glass, PDMS and plastic (Borosilicate Glass about 1.14 W/mK, PDMS about 0.15 W/mK, plastic <1 W/mK).

Furthermore, metals are reflective. The heat transfer sealing layer 105 may therefore reflect fluorescence light emitted by a sample and enable more light to be collected by the detection module.

In an embodiment, the adhesive layer 103 is less than about 200 μm in thickness. In another embodiment, the adhesive layer 103 may be about 100 μm or less. In yet another embodiment, the adhesive layer 103 may be about 50 μm or less. In a further embodiment, the adhesive layer 103 may be about 28 μm or less. Specifically, the adhesive layer 103 may be as thin as about 0.1 μm and this may be the lower limit for all possible embodiments discussed above. In particular, the adhesive layer 103 may be as thin as possible while ensuring adequate bonding and ensuring that the surface is smooth.

Examples of commercially available materials suitable for forming the adhesive layer 103 include, but are not limited to acrylic, silicone, rubber, polyester, etc. In particular, the adhesive may be chosen to be compatible with the liquid inside the microchannel 101 and the heat conditions according to application.

For example, for use in PCR, the adhesive layer 103 may be DNAse/RNAse/nucleic acid free and heat resistant (in the range of at least from about 25° C. to about 100° C.). Use of the embodiment shown in FIG. 1(a) in PCR reactions is discussed further below. Thermally conductive films fabricated with adhesive layers suitable for PCR reactions are commercially available.

For droplet-based DNA transformation (discussed further below), as the reagents and bacterial cells are encapsulated in a drop, the nature of adhesive material is not particularly limited. However, the heat cycle according to literature protocols includes cooling the sample to 4 degrees centigrade, so the adhesive may be chosen to function adequately under cold conditions.

For some applications, the channel walls may particularly be hydrophilic or hydrophobic and therefore the adhesive chosen may be correspondingly hydrophilic or hydrophobic. Suitable adhesive materials are commercially available.

Advantageously, inclusion of an adhesive layer 103 in the heat transfer sealing layer 105 according to this embodiment enables straightforward manufacture. In embodiments, other bonding methods (such as chemical bonding) may be employed instead of using the adhesive layer 103 i.e. the heat transfer sealing layer 105 may not include an adhesive layer 103. Advantageously, these other methods of bonding may produce thin bonding layers, thereby enabling precise temperature control by minimizing the thickness of material between the thermally active element 107 and the microchannel 101.

The microfluidic chip 1 may also comprise (not shown) a spacer plate carrying or housing the thermally active element 107; a support plate to provide support for the microfluidic chip 1; a fastening element to attach the main channel plate 113 to the spacer plate and the support plate with the thermally active element 107 sandwiched between the main channel plate 113 and the support plate. The fastening element may be a bolt and a nut, or a snap lock pin and a spring. In particular, the fastening element may be a magnet to allow for self-alignment and quick attachment and release of the channel plate with the spacer plate. Employing magnets as the fastening element is advantageous in the case the main channel plate 113 and heat transfer sealing layer 105 are required to be quickly detached from the spacer plate so that a new main channel plate 113 and heat transfer sealing layer 105 for use with a different liquid or sample such as a clinical sample from a different patient can be attached and used on the microfluidic chip 1, in exactly the same way as the original main channel plate 113 and heat transfer sealing layer 105.

In an embodiment, the main channel plate 113 may further comprise one or more sensor slots 111 for installing a temperature sensor for temperature feedback control to the thermally active element. In the embodiment of FIG. 1(a), the sensor slot passes through both the heat transfer sealing layer 105 and such that a sensor received in the sensor slot 111 is in direct contact with the surface of the thermally active element 107. For example, if the heat transfer sealing layer 105 includes a film, the sensor may be in direct contact with the surface of the thermally active element via a hole through the film and the adhesive layer 103. The portion of the adhesive layer 103 at the position of the temperature feedback sensor can be removed from the heat transfer sealing layer 105 so that the temperature feedback sensor can be placed directly in contact with the surface of the thermally active element 107.

Advantageously, the direct contact between the sensor and the thermally active element 107 enables precise control of the thermally active element 107 without a lag in the temperature response due to a heat gradient caused by intervening materials between the sensor and thermally active element 107.

In other embodiments, the sensor slot 111 is positioned such that a sensor received within the sensor slot 111 is in direct contact with the adhesive layer 103 of the heat transfer sealing layer 105, as shown in FIG. 1(b). In embodiments in which a thin film is employed in the heat transfer sealing layer 105, particularly a metallic film, the thinness of the materials ensures that the temperature lag between the thermally active element 107 and the opposing side of the heat transfer sealing layer 105 will be minimal. As the surface of the heat transfer sealing layer 105 (optionally including the adhesive layer 103) is in direct contact, i.e. contiguous, with the sample received in the microchannel 101, a sensor received in the sensor slot 111 in this configuration will give an accurate indication of the temperature at the surface of the sample.

In some embodiments, for example, those employing a thin plate as the heat transfer sealing layer 105, one or more sensor slots 115 may be situated instead or in addition to the sensor slot 111 within the heat transfer sealing layer 105 itself enabling a sensor to be embedded in the heat transfer sealing layer 105 for measurement (see for example the embodiment of FIG. 1(c)).

FIG. 1(c) shows a microfluidic chip 11 according to another embodiment. In this embodiment, the adhesive layer 103 is selectively removed from the heat transfer sealing layer 105 over the channel area in the region where the transfer sealing layer 105 is in direct contact with the liquid in the microchannel 101. This can be achieved by pre-processing the adhesive film, such as by selective etching of the adhesive following the profile of the microchannel 101 or post-processing the assembled microfluidic chip 11, such as by flushing a suitable solvent for the adhesive through the microchannel 101.

Advantageously, in embodiments in which the heat transfer sealing layer 105 is 0.04 mm or greater selective removal of the adhesive layer 103 over the channel area enables improved temperature control of the sample and reduces the temperature gradient between the thermally active element 107 and the sample, due to a reduction in the thickness of intervening materials. In certain cases (but not all cases), for example when a thin plate is employed as the heat transfer sealing layer 105, the thin plate may contain at least one sensor slot 115 to embed a sensor for measurement, such as for temperature feedback. The adhesive layer 103 may comprise double-sided tape in which adhesive at the channel area is removed by pre-processing, for example by laser cutting, or die cutting, or by post-processing after being attached to the microfluidic chip 11, for example by passing solvent for the adhesive through the microchannel 101.

FIG. 1(d) shows a variation on the embodiment of FIG. 1(c). The arrangement of the microfluidic chip 11 is identical to that of FIG. 1(c) but with a sensor slot 111 passing through the heat transfer 105 layer including the adhesive layer 103 such that a sensor (not shown) received in the sensor slot 111 is positioned on the surface of the thermally active element 107. As the sensor does not need to be embedded in the heat transfer sealing layer 105, this arrangement enables a thinner heat transfer sealing layer 105 to be employed than in the arrangement of FIG. 1(c), thereby enabling improved heat transfer efficiency to the sample. In particular, the heat transfer sealing layer 105 may include a metal film as discussed above in accordance with the embodiment of FIGS. 1(a) and 1(b). Advantageously, this arrangement provides efficient transfer of heat from the thermally active element 107 to a sample received within the microchannel 101.

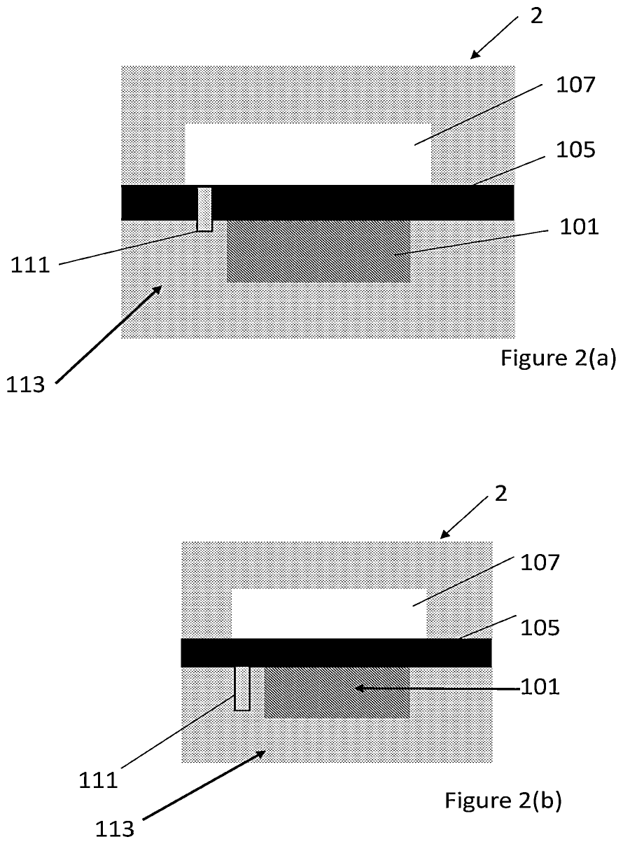

FIG. 2(a) shows a microfluidic chip 2 according to another embodiment. In contrast to the embodiments of FIGS. 1(a)-(d), in this embodiment, the adhesive layer 103 is not employed in the heat transfer sealing layer 105 of microfluidic chip 2. In this embodiment, the microfluidic chip 2 includes: a main channel plate 113 carrying a microchannel profile; a heat transfer sealing layer 105; a thermally active element 107; and a sensor slot 111.

In addition, the device may include a seal, for example an elastomeric seal arranged to surround the channel profile (not shown), similar to an O-ring. The elastomeric seal may be received in a seal groove (not shown) and shaped to match the channel profile. Thus, in this case the heat transfer sealing layer 105 cooperates with the wall of the microchannel profile in the main channel plate 113 via the elastomeric seal.

In an embodiment, the channel plate may further comprise one sensor slot or more 111 for installing a temperature sensor for temperature feedback control to the thermally active element. In the embodiment of FIG. 2(a), the sensor slot 111 passes through the heat transfer sealing layer 105 and is arranged such that a sensor received in the slot is in direct contact with the surface of the thermally active element. For example, if the heat transfer sealing layer 105 is a film, the sensor may be in direct contact with the surface of the thermally active element via a hole through the film.

Advantageously, the direct contact between the sensor and the thermally active element 107 enables precise control of the thermally active element 107 without a lag in the temperature response due to a heat gradient caused by intervening materials between the sensor and the thermally active element.

In another embodiment, as shown in FIG. 2(b), the sensor slot does not pass through the heat transfer sealing layer 105 but is instead positioned on it so that a sensor received within the slot measures the temperature of the surface of the heat transfer sealing layer 105. As the surface of the heat transfer sealing layer 105 is configured to be in direct contact with a sample in the microfluidic channel, the sensor in this configuration will measure the temperature at the surface of the sample.

The requirements and particular features of the heat transfer sealing layer 105 and thermally active element 107 discussed above in relation to FIG. 1(a) are equally applicable to the variations and embodiments of FIGS. 1(b), 1(c), 1(d), 2(a) and 2(b).

In variations of any the embodiments described in FIGS. 1(a), 1(c), 1(d), 2(a) and 2(b) above, for example, those in which a thin plate is employed as the heat transfer sealing layer 105, one or more sensor slots 115 may be situated instead or in addition to the sensor slot 111 within the thin plate enabling a sensor to be embedded for measurement, such as in the sensor slot arrangement shown in FIG. 1(c).

Likewise, as in the embodiment of FIG. 1(a), the microfluidic chips 11, 2 may also comprise (not shown) a spacer plate carrying or housing the thermally active element 107; a support plate to provide support for the microfluidic chips 11, 2; and a fastening element to attach the main channel plate 113 to the spacer plate and the support plate with the thermally active element 107 sandwiched between the main channel plate 113 and the support plate. The fastening element may be a bolt and a nut, or a magnet. In particular, the fastening element is a snap lock pin and a spring. The main channel plate 113 may be disposable while the spacer plate, the thermally active element 107, and the support plate may be reusable, i.e. the main channel plate 113 and heat transfer sealing layer 105 may be replaced with a new channel plate 113 and heat transfer sealing layer 105, for example for use with a different sample to be tested, or for use with an alternative channel profile.

It is envisaged that features relating to one of the embodiments described above may be employed in another embodiment. For example, sealing of the channel using an elastomeric seal, such as or similar to an O-ring may be employed in addition to or instead of employing adhesive in any of the described embodiments. In general, elastomeric sealing such as O-ring sealing is stronger than adhesive bonding. The bonding strength of the adhesive layer depends on the adhesive material and its thickness. For applications with high flow, or with flow over a period of time (such as culture of cells, biofilm etc.), the pressure build-up is high, so an elastomeric seal such as an O-ring may be particularly employed in these embodiments. For static application (such as PCR, incubation) or with low flow and/or over a short time (DNA transformation in a few minutes to hours), adhesive, or the simple fastening mechanism of the microfluidic chip provides enough bonding to prevent leakage and an elastomeric seal such as an O-ring is not required.

Examples of arrangements employing elastomeric seals, such as O-rings according to embodiments will be discussed further below.

Thus, in the embodiments of FIGS. 1(a)-(d) and 2(a) and (b), a heat transfer sealing layer 105 structurally supported by a thermally active element 107 cooperates with a wall of the microchannel 101 in order to enable efficient and uniform heating during thermal cycling of a sample contained therein. In an embodiment, the heat transfer sealing layer may act as the enclosing member 109 of the microchannel 101 itself, thereby enabling direct contact of heat transfer sealing layer 105 to a sample/specimen. This enables efficient and rapid temperature control as a temperature gradient forms across the thin heat transfer sealing layer 105 from thermally active element 107, without any mediation from other materials. Coupled with the high thermal conductivity of the heat transfer sealing layer 105, this enables rapid attainment of a setpoint temperature when isothermal conditions are desired and enables a high heating/cooling rate in applications where cyclic thermal control is employed.

Advantageously, the microfluidic chips 1, 11, 2 of FIGS. 1(a)-(d) and 2(a) and (b) may provide active heating without degradation of sample/specimen. This is because the thermally active element 107 is itself not in contact with the fluid/sample. Furthermore, heat alone is conducted in or removed from the micro channel 101 via the heat transfer sealing layer 105; no other physical effects are applied to the sample material. This means that temperature control according to embodiments may be applied to any sample material and thus applicability of the process is not limited.

Thus, the integration of a heat transfer sealing layer 105 into the wall of the microchannel 101 may enable improved thermal control over the reagents in the microchannel 101.

The temperature control area, temperature control range and heating/cooling rate can be easily changed by changing the specification (including the size and capacity) of the thermally active element 107 and/or heat transfer sealing layer 105. This will become apparent from the detailed embodiments discussed below.

In an embodiment, the microfluidic chips 1, 11, 2 according to FIGS. 1(a)-(d) and 2(a) and (b) are configured to perform isothermal temperature control. In another embodiment, the chips are configured to perform cyclic thermal control. In an embodiment, the chips may perform either.

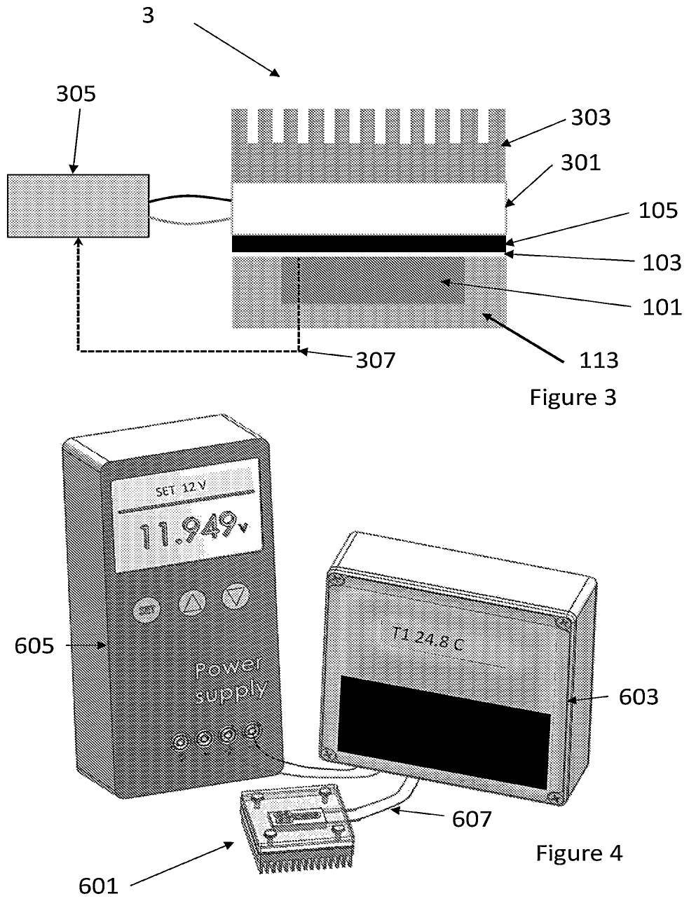

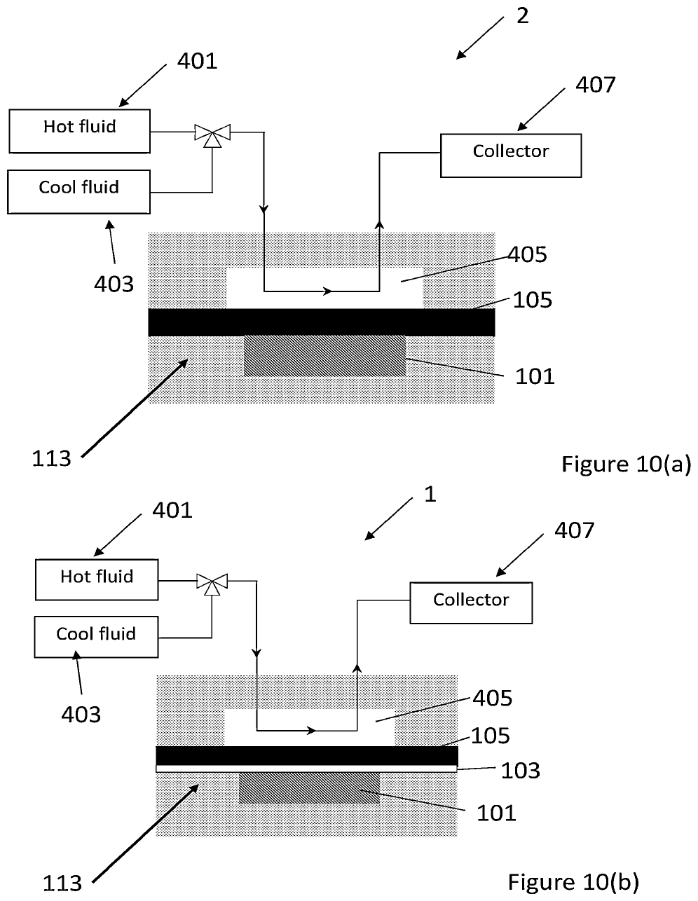

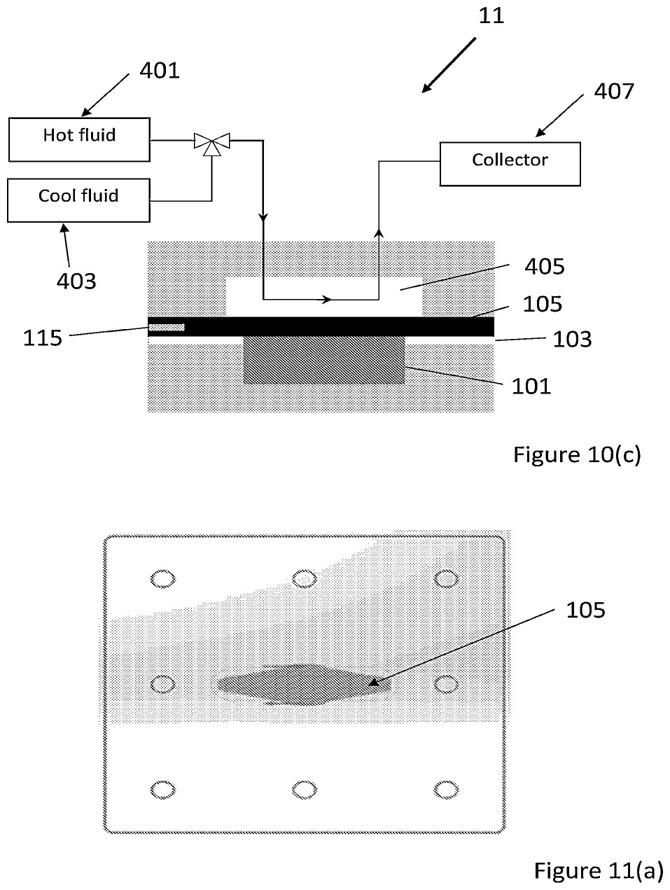

The structure of the microfluidic chips 1, 11, 2 according to the above embodiments enables various types of heat control elements to be employed as the thermally active element 107. In particular, the thermally active element 107 may comprise a thermoelectric heat pump (equivalently thermoelectric device, or Peltier device). An arrangement comprising a thermoelectric heat pump (Peltier device) 301 according to an embodiment is shown in FIG. 3. FIGS. 10(a) to (c) illustrate an alternative embodiment using a pair of hot and cold reservoirs 401 and 403 as the heat source and heat sink, respectively.

In an embodiment, the microfluidic chips 1, 11, 2 according to embodiments discussed above are plastic-based. Plastics offer a wide range of polymeric material selection with a wide range of material properties for various applications. This enables wide range of temperature control, chemical conditions, etc.

Further, plastics are suitable for a wide range of fabrication method options for the microreservoir/microchannel profile (injection moulding, hot embossing, nanoimprinting, micromachining, 3D printing, laser cutting, etc.). Established manufacturing processing can therefore be leveraged for making the microfluidic chips according to embodiments described herein making enabling straightforward integration into existing manufacturing lines and high-volume production. As discussed above, the use of a thermally conductive film in the heat transfer sealing layer also enables straightforward integration into these existing manufacturing methods. Thus, the simple channel structure of the embodiments of FIGS. 1(a)-(d) and 2(a) and (b) may enable straightforward mass production while still enabling temperature control.

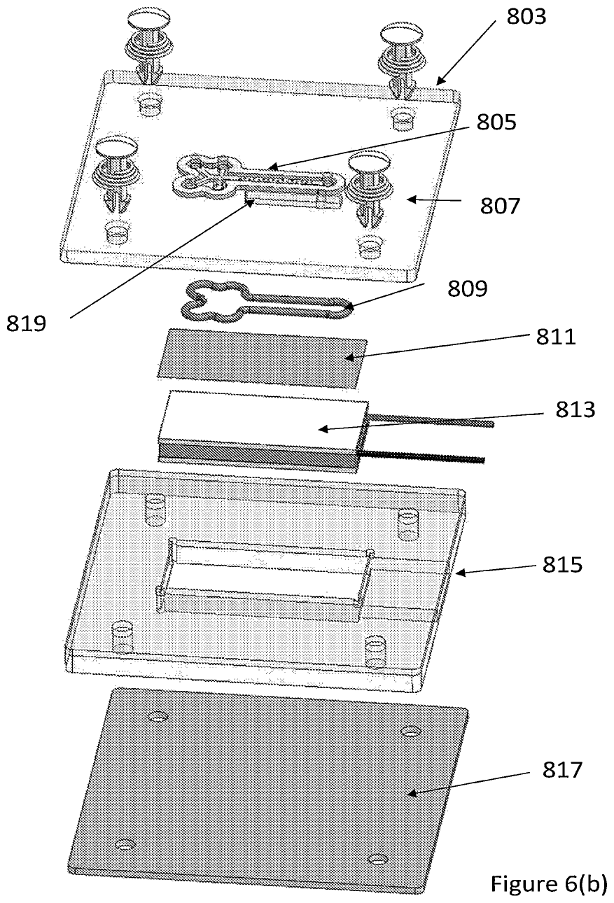

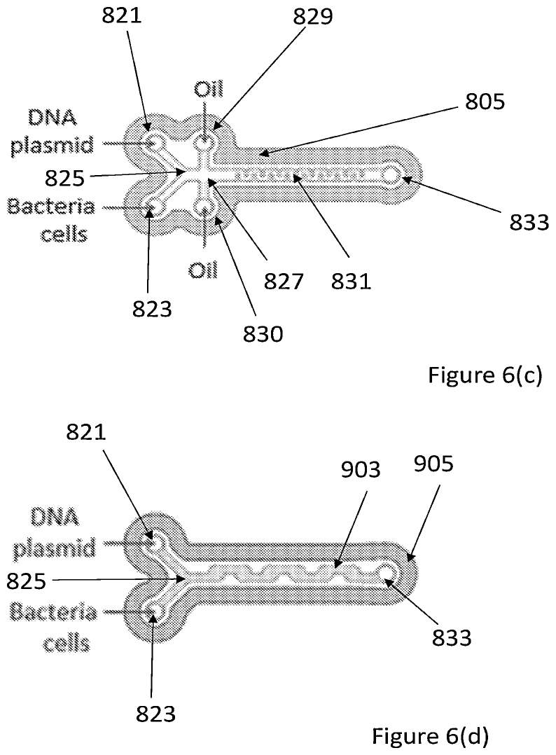

FIG. 3 shows a schematic example of a system according to an embodiment including a microfluidic chip 3 which is an example of the microfluidic chip 1, i.e. is in accordance with the embodiments of FIGS. 1(a) and (b). In this example, the thermally active element 107 is a solid-state thermoelectric heat pump 301 (also known as a Peltier device) backed by a heat sink 303. In the example of FIG. 3, the temperature of the heat transfer sealing layer 105 is controllable by the electric current flow through the thermoelectric heat pump 301. The heat will be conducted to the microchannel 101 through the heat transfer sealing layer 105 including the adhesive layer 103. It is straightforward to switch from cooling to heating of the microchannel 101 by reversing polarity of the DC current flowing through the thermoelectric heat pump 301. The thermoelectric heat pump 301 is placed in direct thermal contact, such as in direct physical contact or via a sandwiched layer of thermal compound or thermal paste, with the heat transfer sealing layer 105. As the thermoelectric heat pump 301 has high hardness, in this embodiment, the heat transfer sealing layer 105 may be as thin necessary to maintain a seal of the channel and/or the channel shape in conjunction with the structural support provided by the backing of the thermoelectric heat pump 301, while enabling handling during manufacture without breakage. Without the structural support of the thermoelectric heat pump 301, the heat transfer sealing layer 105 may not be able to hold the sample to be tested. The structural support provided by the thermoelectric heat pump 301 may enable the heat transfer sealing layer 105 to support the sample to be tested even in cases in which pressure build-up inside the microchannel is high and/or during high-flow or long duration applications. In an embodiment, the heat transfer sealing layer 105 may be less than about 50 μm in thickness, for example, about 36 μm (excluding the adhesive layer 103). The heat transfer sealing layer 105 including adhesive layer 103 (and optional elastomeric seal, such as or similar to an O-ring) backed by the thermoelectric heat pump 301 is then assembled with the main channel plate 113 to form the microchannel structure (examples of which are shown in FIGS. 6, 7 and 8). A heat sink 303 is attached to the side of the thermoelectric heat pump 301 opposite the microchannel 101. The heat sink 303 can be a passive heat sink (for example, a metal block with fins) or an active heat sink (for example, air cooling or liquid cooling), or a combination of both. A power supply and feedback control system 305 is used to control the thermoelectric heat pump 301. A feedback temperature control sensor (not shown) can be installed onto the heat transfer sealing layer 105 and/or the adhesive layer 103 of the heat transfer sealing layer 105 as in FIG. 1(a) or the thermoelectric heat pump surface in order to enable temperature monitoring via connection 307.

A thermoelectric heat pump 301 generates temperature gradient across it when a potential difference is applied. As such, by controlling the magnitude and polarity of the voltage applied across the thermoelectric heat pump 301, bipolar heating and cooling of sample/specimen in the microchannel 101 can be achieved.

In an embodiment, the system of FIG. 3 enables both isothermal and cyclic thermal control with two or more setpoint temperatures. Examples of isothermal operating modes according to embodiments include cooling the microfluidic channel to −20° C. from room temperature and holding the temperature at −20° C. (thereby deep freezing the sample in the microfluidic channel). In another example, the microfluidic chip 3 may be employed to culture bacteria in the microchannel at 37° C. In another example, the microfluidic chip 3 may be employed to perform isothermal of nucleic acid amplification, such as holding the temperature at 65° C. for 30 min during loop-mediated isothermal amplification (LAMP).

The person skilled in the art will appreciate that other isothermal processes could be implemented using microfluidic chips according to embodiments disclosed herein.

Examples of cycling thermal control according to embodiments include performing polymerase chain reaction (PCR) or reverse-transcription polymerase chain reaction (RT-PCR), such as heating to 95° C. for 30 s for initialization; 30 cycles of 95° C. for 10 s (for denaturation of the double-stranded DNA template), 58° C. for 30 s (allowing annealing of the primers to each of the single-stranded DNA template), and 72° C. for 30 s (for extension). Another example of a cyclic thermal control process is that of initiating heat shock in bacteria for transformation processes in which a temperature of a sample/specimen is to be cycled from 4° C. rapidly to 42° C. and then back to 4° C. With at least one temperature feedback sensor, the embodiment in FIG. 3 may be able to perform more than 2 set-point dynamic thermal control, e.g. heating from 0° C. to 37° C. and holding, then rapid cooling to −20° C. and holding. This programme effectively simulates thawing the sample/specimen, then incubating it, followed by deep freezing the sample/specimen. Thus, systems according to embodiments such as that of FIG. 3 are capable of a wide range of application.

The size, shape and capacity of the thermoelectric heat pump 301 may be customizable. The temperature control region may be altered by changing the size and shape of the thermoelectric heat pump 301 enabling localized heating/cooling in the microfluidic channel 101. Localized control may be achieved by using a smaller thermoelectric heat pump 301 which covers only a segment, or portion of the microchannel 101. The intersection area between the thermoelectric heat pump 301 and the microchannel 101 may be the intended region for localized control. Furthermore, a wide temperature range may be achieved by using a thermoelectric heat pump 301 with different capacity. In an embodiment, two or more thermoelectric heat pumps may be stacked to enable further increases in achievable temperature range.

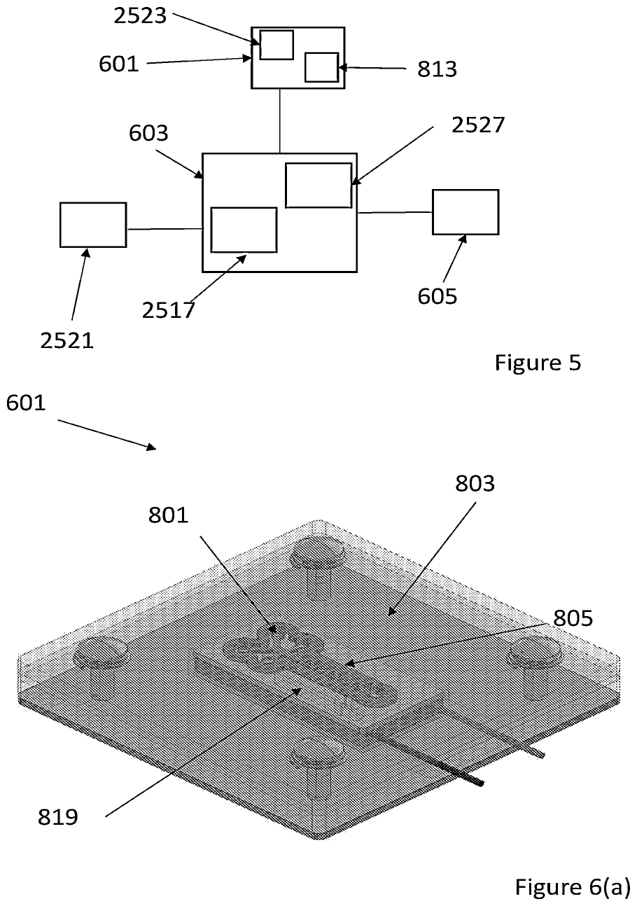

FIG. 4 illustrates a system according to an embodiment having a similar configuration as the system of FIG. 3, comprising a microfluidic chip 601 which is an example of the microfluidic chip 2 of the embodiment of FIG. 2(b), i.e. does not include an adhesive layer 103. Instead, an elastomeric seal around the microchannel profile in the microfluidic chip 601 is provided. FIG. 5 shows a detailed schematic representation of the system of FIG. 4. The microfluidic chip 601 comprises a thermoelectric heat pump 813, the thermoelectric heat pump 813 being electrically connected via electrical wires 607 to temperature feedback controller 603 for controlling the operation of the thermoelectric heat pump 813 itself and a power supply unit 605 connected to the temperature feedback controller 603 (together equivalent to the power supply and feedback control system 305 of FIG. 3) providing power to both the thermoelectric heat pump 813 and the temperature feedback controller 603. The system may also include an optional laptop or computer (not shown) for data logging.

FIG. 5 shows a more detailed schematic representation of the system of FIG. 4. The microfluidic chip 601 comprises temperature sensor 2523 connected to the temperature feedback controller 603. The temperature feedback controller 603 is configured to receive temperature data from the temperature sensor 2523 and control power supply to the thermoelectric heat pump 813 of the microfluidic chip 601. The temperature feedback controller 603 may comprise a processor 2517 configured to process the temperature data received from the temperature sensor 2523 and control the thermoelectric heat pump 813 accordingly. The temperature feedback controller 603 may comprise a memory 2527 configured to store set points for temperature control of the microfluidic chip 601 and/or a temperature control program to be performed by the processor 2517. The temperature feedback controller 603 may be connected to at least one power supply, such as power supply unit 605. The temperature feedback controller 603 may be connected to an input 2521 configured to receive inputs from a user or a network, for example, setpoint temperatures for the microfluidic chip 601, etc.

FIGS. 6(a) and 6(b) illustrate the chip 601 in detail (with heat sink 303 omitted for clarity).

In this embodiment, the chip comprises a single channel 801 suitable as a droplet-based fluidic mixer for on-chip DNA transformation. A detail of the channel profile is shown in FIG. 6(c).

In this embodiment, due to only the presence of a single channel 801 on the microfluidic chip a thermoelectric heat pump 813 with a relatively small surface area may be used. The main channel plate 803 contains the single channel 801 and a seal groove 805 that receives an elastomeric seal element, in this case an elastomeric seal 809 similar to an O-ring that is configured to be seated in the seal groove 805. In an embodiment, the elastomeric seal 809 is circular (as are widely commercially available) but is shaped manually or otherwise into the seal groove. In other embodiments, the elastomeric seal 809 may be custom made to match the shape of the seal groove 805.

The elastomeric seal 809 thus received in the seal groove functions as the sealing mechanism for the channel 101. The heat transfer sealing layer 811, comprising, for example, a copper film, and structurally supported by the thermoelectric heat pump 813 is secured onto the elastomeric seal 809 forming the sealed microchannel. The thermoelectric heat pump 813 is seated in a spacer plate 815 that is backed by a conductive support plate 817 which may be connected to a heat sink. The assembly can be secured by using a range of fastening mechanisms 807 including, but not limited to, snap-lock pins with spring elements, bolts and nuts, and magnets. The securing of the assembly in this way ensures the elastomeric seal 809 is secured onto the thermoelectric heat pump 813. Without the backing of the thermoelectric heat pump 813, the heat transfer sealing layer 811 may not be able to hold the sample to be tested in the single channel 801.

In this embodiment the sensor slot 819 does not pass through the heat transfer sealing layer 811, and therefore a temperature sensor received within the sensor slot 819 sits on the surface of the heat transfer sealing layer 811. In other embodiments, in accordance with the arrangement shown in, for example FIGS. 1(a) and 2(a), the sensor slot 819 may pass through the heat transfer sealing layer 811 so that a sensor received in the sensor slot 819 sits on the surface of the thermoelectric heat pump 813. This may be achieved by, for example, a hole in the heat transfer sealing layer 811.

FIG. 6(c) illustrates the channel profile of the channel 801. The channel profile comprises four inlets 821, 823, 829, 830, a periodically oscillating, or serpentine section, 831 and two junctions 825 and 827 joining the four inlets 821, 823, 829, 830 to the periodically oscillating section 831 via linear (straight) channels leading from each inlet. The periodically oscillating section 831 is further connected to an outlet 833 comprising a through-hole through the channel plate 803. Inlets 821 and 823 are connected via Y-junction 825 which, in turn is connected to inlets 829, 830 via cross junction 827 which is itself connected to periodically oscillating section 831. When employed as a droplet-based fluidic mixer for on-chip DNA transformation, DNA plasmid is introduced to the channel 801 via inlet 821, competent cells such as bacteria cells or yeast cells via inlet 823 and oil via inlets 829, 830. The seal grove 805 formed so as to completely surround and outline the shape of the channel profile is also shown in FIG. 6(c).

FIG. 6(d) shows an alternative channel profile which could be employed as a mixer. The channel profile is broadly similar to that of FIG. 6(c), comprising a periodically oscillating section 903 (albeit with oscillations which are more elongate than that of FIG. 6(c)) leading to an outlet 833 and inlets 821 and 823 fluidically connected to the periodically oscillating section 903 via junction 825. Oil inlets 829 and 830, however are omitted and therefore only one junction 825 is required. A seal grove 905 formed so as to completely surround and outline the shape of the channel profile is also shown in FIG. 6(d).

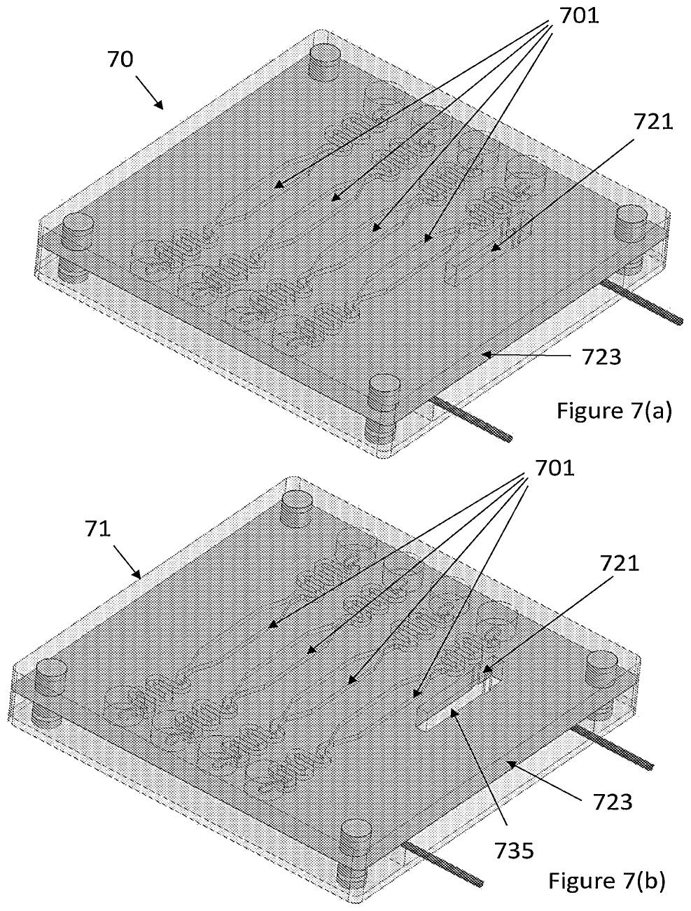

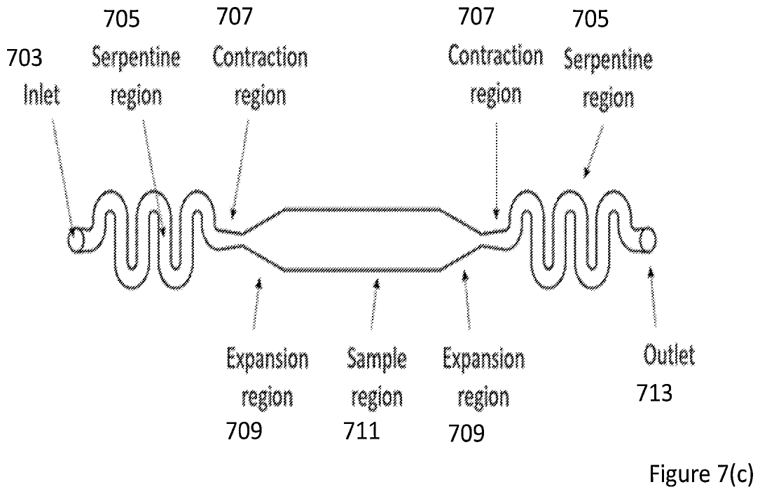

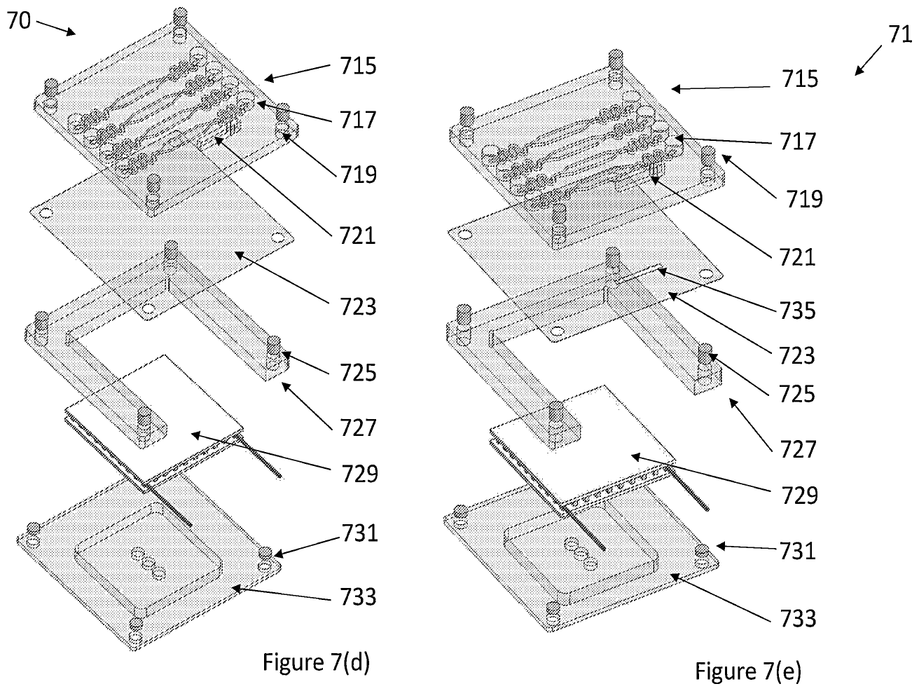

FIGS. 7(a) and 7(b) show a perspective view of two alternative microfluidic chips 70, 71 which are both examples of microfluidic chip 1, i.e. are in accordance with the embodiments of FIGS. 1(b) and (a), respectively. Both microfluidic chips 70, 71 could be employed with the platform of FIG. 4. Microfluidic chips 70, 71 include larger thermoelectric heat pumps than that employed in the embodiment of FIGS. 6(a)-(c).

Both microfluidic chips 70, 71 comprise 4 PCR (polymerase chain reaction) channels 701. In other embodiments the microchips 70, 71 may have five or six or more PCR channels 701. In other embodiments the microchips 70, 71 may have fewer PCR channels 701. The number of PCR channels 701 is not limited to six channels and can be further extended to increase the number of concurrent samples that can be processed. Each PCR channel 701 may be used as an independent reaction chamber.

In addition, both microfluidic chips 70, 71 comprise temperature sensor slots 721. In the embodiment of FIG. 7(a), the temperature sensor slot 721 is configured such that a temperature sensor received in the temperature sensor slot 721 sits on the heat transfer sealing layer 723, as described above in association with FIG. 1(b). In this case, a sensor received in the temperature sensor slot 721 will measure the temperature of the heat transfer sealing layer, i.e. the surface temperature of the liquid in the channel. In contrast, in the embodiment of FIG. 7(b), the temperature slot is configured to pass through the heat transfer sealing layer 723 so that a temperature sensor received within the slot sits on the surface of the thermoelectric heat pump, as described above in association with FIG. 1(a). In this case, the sensor will measure the temperature of the thermoelectric heat pump.

A detail of the profile of one of the PCR channels 701 formed in the channel plate 715 according to an embodiment is shown in FIG. 7(c). In particular, the PCR channel profile includes one inlet 703 feeding into a serpentine region 705, followed by a contraction region 707 in which the channel profile narrows and an expansion region 709 in which the channel profile widens leading to a main chamber 711 in which the PCR master mix and sample will reside during a PCR reaction, followed by a second expansion region 709—contraction region 707—serpentine region 705 leading to an outlet 713. The serpentine regions 705 act as flow resistors. During a PCR reaction, in some protocols, temperature of the sample is raised to over 90° C. As such, the vapor pressure of liquid sample is high and it can start to flow out of the channel. The serpentine regions 705 prevent the sample from escaping from the PCR channel 701 and undergoing evaporation.

Advantageously, the PCR channels 701 are loaded using an oil sandwiching method comprising loading an initial quantity of oil, followed by the sample, followed by another quantity of oil. Examples of suitable oils include but are not limited to mineral oil and silicone oil. The flow resistors in the form of the serpentine regions 705 according to the embodiment of FIG. 7(c) work in tandem with this sample loading method of sandwiching the sample with oil by preventing the sample from escaping.

An exploded view of the assemblies of the microfluidic chips 70, 71 is illustrated in FIGS. 7(d) and (e), respectively.

In both microfluidic chips 70, 71, the channel plate 715 comprises four PCR channels 701 with the form shown in detail in FIG. 7(c). Additionally, there is a small reservoir 717 at each of the inlets 703 and outlets 713 that acts as a catchment to contain fluid (oil, etc.) that might run out from the PCR channel 701 due to the vapor pressure caused by high temperatures. The channel plate 715 has one sensor slot 721 or more, for installing a temperature sensor to provide temperature feedback control. The heat transfer sealing layer 723 (for example, an aluminium film with a thin adhesive layer) backed, and therefore supported by the thermoelectric heat pump 729 is secured (by the adhesive) onto the channel plate 715 forming the sealed PCR channel 701. Without the structural support of the thermoelectric heat pump 729, the heat transfer sealing layer 723 may not be able to hold the sample to be tested. The heat transfer sealing layer 723, in this embodiment comprising, for example, a highly conductive film, may enable fast temperature cycling as required by some PCR protocols. In addition, the aluminium film is reflective and aids in enhancing the signal that can be collected by the detection module. Light emitted by fluorescent samples will be reflected back up to the detection module, and as such increase the signal that can be collected. Thermoelectric heat pump 729 is seated in a spacer plate 727 that is backed by a thermally conductive support plate 733 (such as a copper plate, or an aluminium plate) which can be connected to a heat sink. The spacer plate 727 may be made of non-conductive material such as plastics (ABS, PMMA, COC, PC, etc.). The assembly is secured using three sets of magnets 719, 725, 731. Alternatively, snap-lock pins with spring elements; bolts and nuts, etc could be employed. Using magnets in the set of fastening elements 719 securing the channel plate 715 in particular enables self-aligned quick assembly and disassembly, which may enable straightforward replacement of the channel plate and heat transfer sealing layer, for example for use with another sample.

In the embodiment of FIG. 7(e) (which is the exploded view of the microfluidic chip 71 shown in FIG. 7(b)) the conductive/adhesive layer 723 has a hole 735 enabling the sensor to pass through this layer, as explained above. In contrast, in the embodiment of FIG. 7(d) (which is the exploded view of the microfluidic chip 71 shown in FIG. 7(a)), no such hole is present in the conductive/adhesive layer 723.

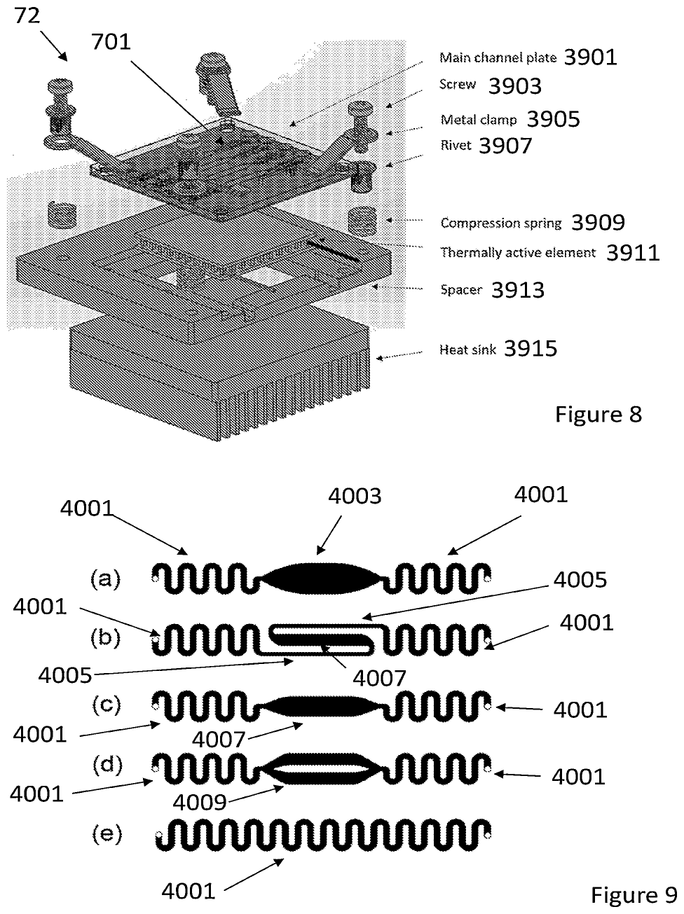

FIG. 8 shows an exploded view of another microfluidic chip 72 which is an example of the microfluidic chip 1, i.e. in accordance with the embodiment of FIG. 1(a) suitable for use with the platform of FIG. 4, including the assembly and mounting. As in the embodiments of FIGS. 7(a) and 7(b), microfluidic chip 72 comprises a channel plate assembly 3901 with a heat transfer sealing layer in the form of a conductive film and an adhesive layer (in FIG. 8, the conductive film with adhesive layer are shown as already affixed to the channel plate), and thermoelectric (Peltier) device 3911, as in the embodiment of FIGS. 7(a) and 7(b) seated in a spacer plate 3913. The spacer 3913 and thermoelectric heat pump 3911 are arranged on a heat sink 3915, the spacer plate 3913 being configured to support the thermoelectric heat pump 3911 on the heat sink 3915. The spacer plate 3913 may be made of non-conductive material such as plastics (ABS, PMMA, COC, PC, etc.). The channel design of the channel plate assembly 3901 is the same as that in of the embodiments of FIGS. 7(a) and 7(b), i.e. 4 PCR channels 701 (polymerase chain reaction channels) as described above in relation to FIG. 7(c).

In this embodiment, the main channel plate 3901 is mounted and secured onto the thermoelectric heat pump 3911 using 2 or more compression-spring-loaded clips, in the embodiment of FIG. 8 each formed from a screw 3903, metal clamp 3905, rivet 3907 and a compression spring 3909. In particular, the main channel plate assembly 3901 may be secured with 4 compression-spring-loaded clips, as in FIG. 8.

Compression-spring-loaded clips may advantageously self-balance the force applied onto the chip thereby ensuring evenly distributed contact force of the heat transfer sealing layer of the chip on the thermally active element (thermoelectric heat pump 3911, in the embodiment of FIG. 8), thereby structurally supporting the heat transfer sealing layer. In addition, the self-balancing nature of the configuration of the spring-loaded clips may ensure even contact of the thermally active element to the heat sink 3915. This self-balancing effect that ensures even contact between the main channel plate 3901, thermoelectric heat pump 3911 and heat sink 3915 and assist in enhancing the uniformity and stability of the temperature distribution across the microfluidic chip 72. The self-balancing effect may also correct for any minor manufacturing variations present in the microfluidic chip 72. Furthermore, the compression-spring-loaded clips enable easy and quick attaching and detaching the main channel plate 3901 onto the thermoelectric heat pump 3911. An optional layer of thermal compound or thermal paste may be applied between the contacting surface of main channel plate 3901 (including the heat transfer sealing layer) to the thermoelectric heat pump 3911 and between the contacting surface of the thermoelectric heat pump 3911 to the heat sink 3915 to enhance heat transfer.

FIGS. 9(a)-(e) show some alternative microfluidic channel designs suitable for PCR reactions according to embodiments, enabling different functionalities according to requirements. In embodiments, these designs can cater to samples of different volume, such as from 10 to 25 μl and may be customized by varying the depth and width of the channels to adapt to different volumes. As in the case of the PCR channel 701, these channels can work in tandem with the sample loading method of sandwiching the sample with oil as discussed above. All designs include periodic serpentine regions 4001 which may function as flow resistors.

FIG. 9(a) shows a channel profile design according to an embodiment consisting of a periodically oscillating serpentine region 4001, followed by a curved, tapered expansion and curved, tapered contraction region which forms an eye-shaped reaction chamber 4003, followed by another serpentine region 4001. In this design, the serpentine regions increase fluidic resistance to prevent samples and the reaction mix from flowing out of the reaction chamber when temperature is raised causing increases in vapor pressure. The serpentine region 4001 also acts as a mixer to enhance mixing of the reagents when being injected into the reaction chamber. The eye-shaped reaction chamber 4003, with its curvature along with the presence of oil may advantageously prevent the sample from breaking up into smaller portions under some temperature cycling conditions.

FIG. 9(b) shows another channel design according to an embodiment. The design consists of a serpentine region 4001, feeding into a narrower long and straight channel 4005, expanding into the wider reaction chamber 4007, followed by a narrower long and straight channel 4005 and finally another serpentine region 4001. This forms an S-shaped channel with the reaction chamber 4007 being in the middle of the S-shape, i.e. the channels 4005, 4007 form a region in which the channel profiles oscillates in a direction perpendicular to the serpentine regions 4001. The serpentine regions 4001 function as flow resistors. It also acts as a mixer to enhance mixing of the reagents when being injected into the reaction chamber. The narrower long and straight channels 4005 that precede and succeed the reaction chamber 4007 acts as a second pair of flow resistors. In combination with the loading method of sandwiching sample in oil (see above), this design is effective in ensuring that the sample stays in the reaction chamber when temperature is raised and cycled.

FIG. 9(c) shows a channel design according to an embodiment consisting of a serpentine region 4001, followed by a tapered region leading into the straight reaction chamber 4007, succeeded by a further tapered region and then another serpentine region 4001, i.e. the channel profile in the reaction chamber 4007 is wider than that of the serpentine, or oscillating regions. This design is a simplification of the channel design in FIG. 7(c) in that it does not have contraction regions at both ends of the reaction chamber 4007. The channel profile of FIG. 9(c) trades off slight reduction in flow resistance but advantageously provides for improved ease of manufacturing. The serpentine regions 4001 also act as mixers to enhance mixing of the reagents when being injected into the reaction chamber.

FIG. 9(d) shows a channel design according to an embodiment consisting of a serpentine region 4001, feeding into two or more straight-channel reaction chambers 4009 preceded and succeed by tapered regions, respectively, and finally another serpentine region 4001. The serpentine regions 4001 function as flow resistors. The serpentine region also acts as a mixer to enhance mixing of the reagents when being injected into the reaction chamber. The multiplexed straight-channel reaction chamber 4009 is configured to split the sample into multiple portions, in this embodiment two. A reaction chamber according to this embodiment may advantageously be employed to sub-sample the biological sample and run the sub-samples under the same conditions in parallel. Furthermore, the relatively narrower reaction chamber of the embodiment of FIG. 9(d) has higher fluidic resistance thereby increasing the range of temperature under which the sample would remain in the reaction chamber as temperature is raised.

FIG. 9(e) shows a channel design according to an embodiment entirely consisting of a serpentine region as the reaction chamber. In this embodiment, the entire channel profile functions as the flow resistor and the sample is spread along it. The serpentine region also acts as a mixer to enhance mixing of the reagents when being injected into the reaction chamber.

In an embodiment, a microfluidic chip may comprise one or more of the disclosed channel profile designs on a single channel plate. In one such example, a channel plate with channel profile designs according to FIG. 9(c), (e), (b), (a) all present on the single channel plate was fabricated by plastic milling. However, other methods of producing channel plates may be employed in accordance with embodiments, including but not limited to injection moulding, hot embossing nano-imprinting, micro-machining, 3D printing and laser cutting.

The depth of each channel on a single channel plate may be varied in order to cater for different reaction volumes.

In the embodiments described above in FIGS. 6(a) to 8, the channel plates 803, 715, 3901 carrying the channel profiles could be disposable or reusable; and could be mass produced using a number of methods known in the art, for example by injection moulding, hot embossing, etc. Indeed, any of the channel plates 113, 803, 715, 3901 according to embodiments described above along with heat transfer sealing layers 105, 811, 723, in common with those of any of the embodiments described herein may be removed and replaced with a replacement channel plate and a replacement heat transfer sealing layer, either separately or together. In particular, in embodiments in which the heat transfer sealing layer is affixed to the channel plate with adhesive, the channel plate—heat transfer sealing layer assembly may be replaced together by another channel plate—heat transfer sealing layer assembly as a single unit.

In an embodiment, the channel plates described above may be manufactured from a blank of suitable thickness made from a non-conductive material such as an optically transparent material (including but not limited to optically transparent plastics) with or without surface treatment. Examples of suitable materials include but are not limited to polymethyl methacrylate (PMMA), COC, PC, ABS, PS, all olefin polymers, and derivatives of any of these materials. As the skilled person will understand, surface treatment of channels may be required depending on the application to prevent fouling, unintended adsorption of reagents, unintended reaction of reagents with the adhesive layer or the heat transfer sealing layer, etc. Surface treatment can be achieved using processes well known in the art, including but not limited to chemical coating, chemical vapor deposition, hydrophilic/hydrophobic surface modification such as plasma, UV/ozone treatment, silanization, PEGylation, grafting polyelectrolytes, BSA coating etc.

In an embodiment, the channel profiles are laser cut into the blank, using for example CO₂ laser cutting. Alternatively, injection moulding, hot embossing, nano-imprinting, machining (such as milling), 3D printing or any combination of these methods in accordance with methods known in the art may also be employed in order to create the channel plate.

For mass production, plastic injection moulding may particularly be employed. For low volume production, machine methods such as milling or laser cutting may particularly be employed. Advantageously, laser cutting provides a particularly fast way of producing the channel profiles. Advantageously, milling achieves high accuracy and a good surface finish.

In an embodiment, the conductive film is a copper or aluminium film (suitable films are widely commercially available) which may be cut to the required size.

The conductive films may be secured to the channel plates 803, 715 using a suitable adhesive, for example using acrylic-based, silicone-based, rubber-based, polyester-based adhesive. The adhesive itself may be applied by, for example, manual application or lamination.

The spacers 815, 727, 3913 may comprise non-conductive material such as plastic (ABS, PMMA, COC, PC, etc.) and manufactured using, for example, machining (milling and laser cutting, etc.), 3-D printing, or plastic injection moulding.

Suitable thermoelectric heat pumps 813, 729, 3911 are commercially available.

The support plates 817, 733 may comprise a conductive material such as copper or aluminium and manufactured by machining methods such as milling, drilling, 3-D printing.

Suitable temperature sensors for use in accordance with embodiments are commercially available.

Suitable fastening elements employed to hold the microfluidic chip together are not particularly limited and are commercially available.

Suitable heat sinks 3915 are commercially available.

Suitable temperature control 603 and power supply modules 605 for use with microfluidic chips 601, 70, 71, 72 are commercially available.

Thus, the microfluidic chips 601, 70, 71, 72 can be fabricated by using established manufacturing techniques for mass production (e.g. injection moulding), and medium to low volume fabrication (e.g. hot embossing, nano-imprinting, micro-machining, 3D printing, laser cutting, etc.). Producing the microfluidic chips 601, 70, 71, 72 according to these well-known techniques increases the applicability of the microfluidic chips 601, 70, 71, 72. Most components of the microfluidic chips 601, 70, 71, 72 may be reusable which may lower the operational cost of the module.

FIGS. 10(a)-(c) illustrate examples of microfluidic chips 2, 1, 11 according to the embodiments of FIGS. 2, 1(a) and (b), and 1(c) and (d), respectively in which the thermally active element 107 is a fluidic heat exchanger comprising two reservoirs 401, 403, a temperature control channel 405 and a collector 407. Temperature control in the microchannel 101 is thus achieved by pumping hot or cold liquid at pre-determined temperatures from two reservoirs 401,403 into a temperature control channel 405 and subsequently into a collector 407. Heat is conducted into or out of the microchannel 101 depending on the temperature difference between the heat exchanger liquid with the environment in the microchannel 101. Isothermal temperature control is achieved by maintaining the relevant reservoir at a desired setpoint temperature and pumping it through the temperature control channel 405.

The heat transfer sealing layer is supported by the fluidic heat exchanger via the temperature control channel 405, the heat transfer sealing layer 105 (optionally including adhesive layer 103) being backed by the temperature control channel 405.

In an embodiment the reservoirs 401, 403 may be a thermostatic electric water bath or syringe heaters, in which the syringes filled with fluid act as the reservoirs, that is capable of heating and/or cooling (as required). Suitable water baths are commercially available.

Cyclic thermal control is achieved by maintaining the two or more reservoirs 401, 403 of fluids at the desired setpoint temperatures and pumping the liquid from the respective liquid reservoirs 401, 403 controlled through an external valve and pump system into the temperature control channel 405. For example, conditions for heat shock bacteria transformation (discussed above) can be created by maintaining two temperature reservoirs 401, 403 hot and cold, respectively and switching the flow of fluid into the temperature control channel 405 from the cold liquid to the hot liquid and then back to the cold liquid. Multi-step and multi-setpoint dynamic temperature control can be similarly achieved by employing as many temperature reservoirs 401, 403 as the desired setpoints.

The heat transfer sealing layer 105 in microfluidic chips according to this embodiment, comprising a fluidic heat exchanger, may comprise a film or a plate. In an embodiment, the heat transfer sealing layer 105 (excluding any adhesive layer 103) may be less than 2000 μm in thickness. The heat transfer layer 105 may be less than about 200 μm in thickness, particularly less than about 50 μm in thickness. In an embodiment, the heat transfer layer 105 may be about 36 μm or less. In an embodiment, it may be as thin as about 0.001 μm.

Likewise, an adhesive layer 103 may or may not be employed to secure the heat transfer sealing layer 105 to the main channel plate 113, as described above in accordance with embodiments. As discussed above in relation to FIGS. 1 and 2, the microfluidic chips 2, 11 and 1 comprise three different arrangements of adhesive according to embodiments. In FIG. 10(a), no adhesive layer is employed. In FIG. 10(b) there is an adhesive layer 103 included in the heat transfer sealing layer 105. In FIG. 10(c) there is an adhesive layer 103 included in a portion of the heat transfer sealing layer 105, however, it is not present on the portion of the chip comprising the microchannel 101. The thickness and properties of the adhesive layer 103 discussed above in relation to the embodiment of FIG. 1 are equally applicable to embodiments employing a fluidic heat exchanger, therefore for brevity will not be repeated here.

As described above in relation to FIGS. 1(a)-(c) and 2(a) and (b), the microfluidic chips 1, 11, 2 according to the embodiments of FIG. 10 may or may not comprise sensor slots 111, 115 for installing a temperature sensor for temperature feedback control of the reservoirs. In the embodiment shown in FIG. 10(c), the sensor slot 115 is located within the heat transfer sealing layer 105 itself. In other embodiments, a sensor slot 111 may pass through the heat transfer sealing layer 105, as shown, for example, in FIGS. 1(a) and 2(a).

In the embodiments of FIG. 10(a)-(c), localized temperature control may be achieved by modifying the area profile of the heat transfer sealing layer 105 and/or the temperature control channel. In an embodiment, the temperature control area may be determined by the intersection area of the heat transfer sealing layer 105 and the main channel 101.

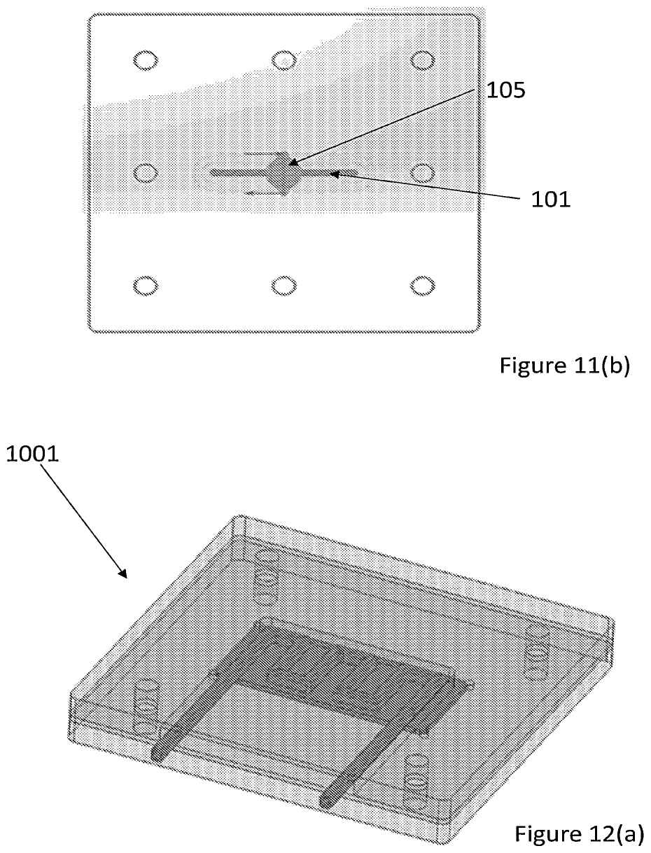

FIGS. 11(a) and (b) show plan views of two arrangements of a microfluidic channel 101 and heat transfer sealing layer 105 according to embodiments. FIG. 11(a) illustrates full control over the entire microchannel 101 with the heat transfer sealing layer 105 covering the entire microchannel 101 (hence the microchannel 101 is not visible). FIG. 11(b), in contrast, illustrates the case where the heat transfer sealing layer 105 only covers a small area of the microchannel 101. Thus, a temperature control channel is defined within the microchannel 101 at the area of intersection between the microchannel 101 and the heat transfer sealing layer 105. This feature will further be illustrated by embodiments discussed below.

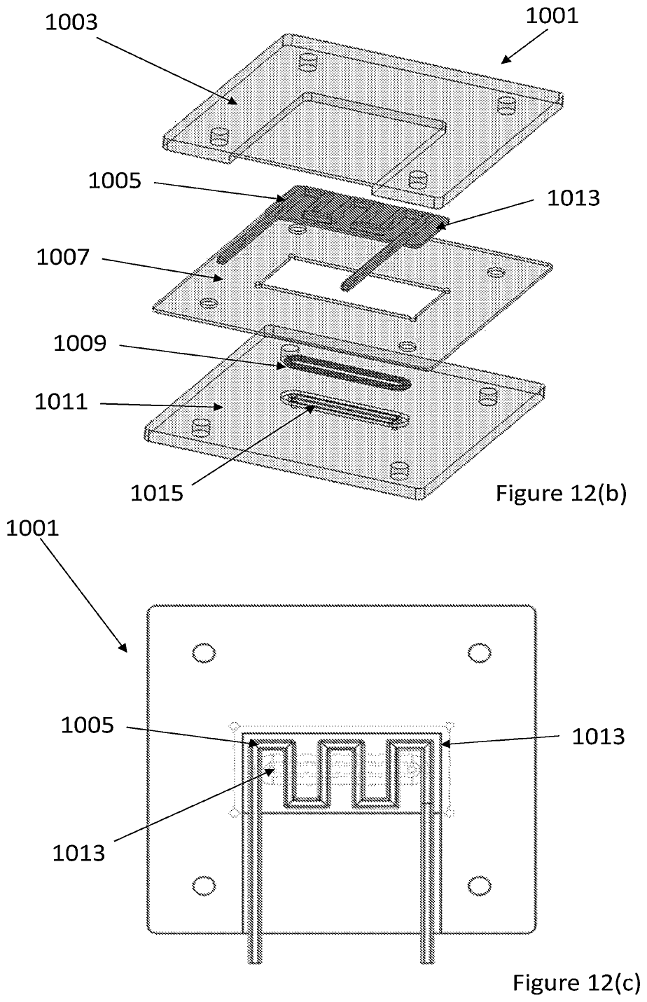

FIGS. 12(a) to (c) illustrate a microfluidic chip 1001 which is an example of microfluidic chip 2 (i.e. in accordance with the embodiment of FIGS. 2(a) and 2(b)) and incorporates a fluidic heat exchanger with full temperature control over the channel according to an embodiment, with FIG. 12(a) showing a perspective view of the assembled microfluidic chip 1001, FIG. 12(b) showing an exploded view and FIG. 12(c) showing a plan view of the assembled microfluidic chip 1001. The microfluidic chip 1001 includes an elastomeric seal surrounding the channel profile.

In this embodiment, the temperature control channel is a metallic heat pipe 1005 that is either directly in contact with the heat transfer sealing layer 1013 or is fabricated as a single piece element (i.e. so as to be integral) with the heat transfer sealing layer 1013, for example by 3-D printing.

The metallic heat pipe 1005 is connected to one or more reservoirs (not shown) of hot and/or cold water or other fluid though an optional one or more valves, according to requirements.

The microfluidic chip 1001 comprises, in addition to the main channel plate 1011 comprising a single, straight channel profile 1015, a heat channel comprising the heat pipe 1005 supporting the heat transfer sealing layer 1013, a spacer plate 1007 and a backing plate 1003. An elastomeric seal in the form of O-ring 1009 is provided in a seal groove around the channel profile 1015 to form a seal between the heat transfer sealing layer 1013 and the channel plate 1011. In this embodiment, the heat transfer sealing layer is a plate. In other embodiments, a film may be employed and the metallic heat pipe 1005 (forming the temperature control channel 405) provide structural support to the film to cooperate with the channel profile 1015 to form the microfluidic reservoir. Optionally, adhesive may be employed to form the seal. The heat transfer sealing layer 1013 covers the entire channel profile 1015 and is seated in the spacer plate 1007 for assembly.

In particular, the heat pipe 1005 may comprise copper or aluminium. Suitable pipes are commercially available.

The microfluidic chip 1001 further comprises fastening elements (not shown). These are not particularly limited and may include (but are not limited to) a bolt and a nut; a snap lock pin and a spring; and a magnet. In particular, the fastening elements may comprise a snap lock and spring.

In an embodiment, the channel plate 1011 may be manufactured from a blank of suitable thickness according to requirements made from a non-conductive material such as ABS, polymethyl methacrylate (PMMA), COC, PC, etc. In an embodiment, the channel profiles are laser cut into the blank, using for example using CO₂ laser cutting. Alternatively, injection moulding, hot embossing, nano-imprinting, machining such as milling, 3D printing or any combination of these methods in accordance with methods well known in the art may also be employed in order to create the channel plate.

In an embodiment, the heat transfer sealing layer 1013 and pipe 1005 are formed using by 3-D printing of high thermal conductivity materials such as aluminium, copper, etc.

The spacer 1007 and backing plates 1003 may comprise non-conductive materials such as plastic (ABS, PMMA, COC, PC, etc.) and manufactured using, for example, laser cutting, machining such as milling, 3-D printing or plastic injection moulding.

The elastomeric seal 1009 similar to an O-ring may be produced from fluorocarbon (such as Viton™), rubber, neoprene, etc. using moulding methods such as extrusion and secured in place with the elastomeric seal groove. Commercially available elastomeric seals may be employed, or custom-made rings may be used according to requirements.

In an embodiment, the channel plate 1011 may further comprise one sensor slot or more (not shown) for installing a temperature sensor for temperature feedback control to the valves controlling the flow of fluid from the one or more reservoirs. In an embodiment, the sensor slot may be arranged such that a sensor received in the slot is in direct contact with the surface of the metallic heat pipes 1005.

In other embodiments, the sensor slot is positioned such that a sensor received within the slot is in direct contact with the heat transfer sealing layer 1013.

As discussed above, the reservoirs (not shown) may be a thermostatic electric water or syringe heaters, in which the syringes filled with fluid act as the reservoirs, i.e. baths capable of heating and/or cooling (as required). Suitable water baths and syringe heaters are commercially available.

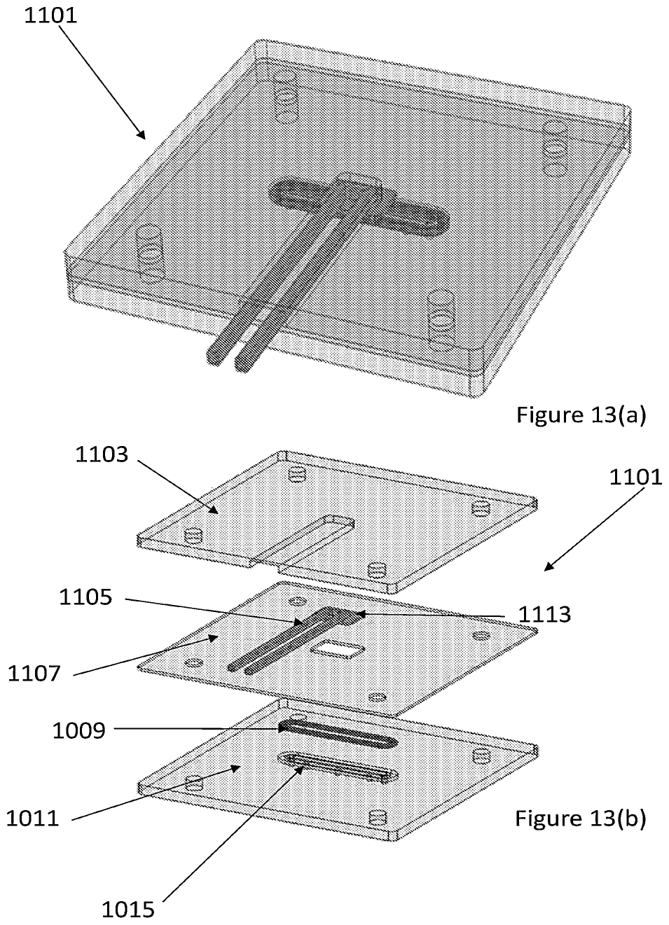

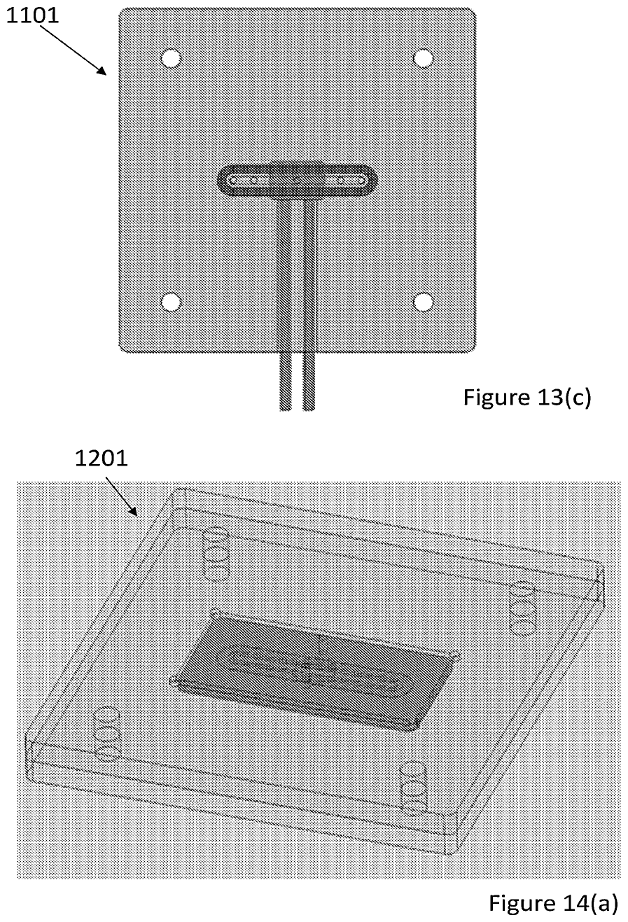

FIGS. 13(a)-(c) show perspective, exploded and plan views respectively of a microfluidic chip 1101 according to an embodiment having the same channel plate 1011 as that of FIG. 12 and comprising a fluidic heat exchanger with a metallic heat pipe 1105 supporting a heat transfer sealing layer 1113 that intersects with only a portion of the channel 1015 in order to enable localised heating of the channel 1015. As before, the heat pipe 1105 and heat transfer sealing layer 1113 are seated in a spacer plate 1107 for assembly. In addition to the main channel plate 1011, the chip 1101 further comprises a spacer plate 1107 and a backing plate 1103. The microfluidic channel, or equivalently microfluidic reservoir is formed by the channel profile 1015 in the main channel plate 1011 and the heat transfer sealing layer 1113. The elastomeric 1009 forms a seal around the main channel 1015 with the heat transfer sealing layer 1113 and the spacer 1107 (with the spacer 1107 providing the backing necessary to provide sealing for the area where the heat transfer sealing layer 1113 does not intersect with the channel 1015). As before, the temperature control channel 1105 is a metallic heat pipe that is directly in contact with the heat transfer sealing layer 1113 or is fabricated as a single piece element with the heat transfer sealing layer 1113, for example by 3-D printing.

The microfluidic chip 1101 according to this embodiment may be produced in the same way as that of FIG. 12, described above, with the size of the components and design of the spacer 1107 adjusted accordingly. The microfluidic chip 1101 has the same configuration as microfluidic chip 2 in FIG. 10(a).

Thus, FIGS. 13(a)-(c) show a fluidic heat exchanger configured for partial heating of the microfluidic channel, in an embodiment, the fluidic heat exchanger in this arrangement could be replaced by a small thermoelectric pump (Peltier device) in direct thermal contact and supporting the heat transfer sealing layer 1113. This arrangement would therefore enable partial heating of the microfluidic channel using a thermoelectric pump.

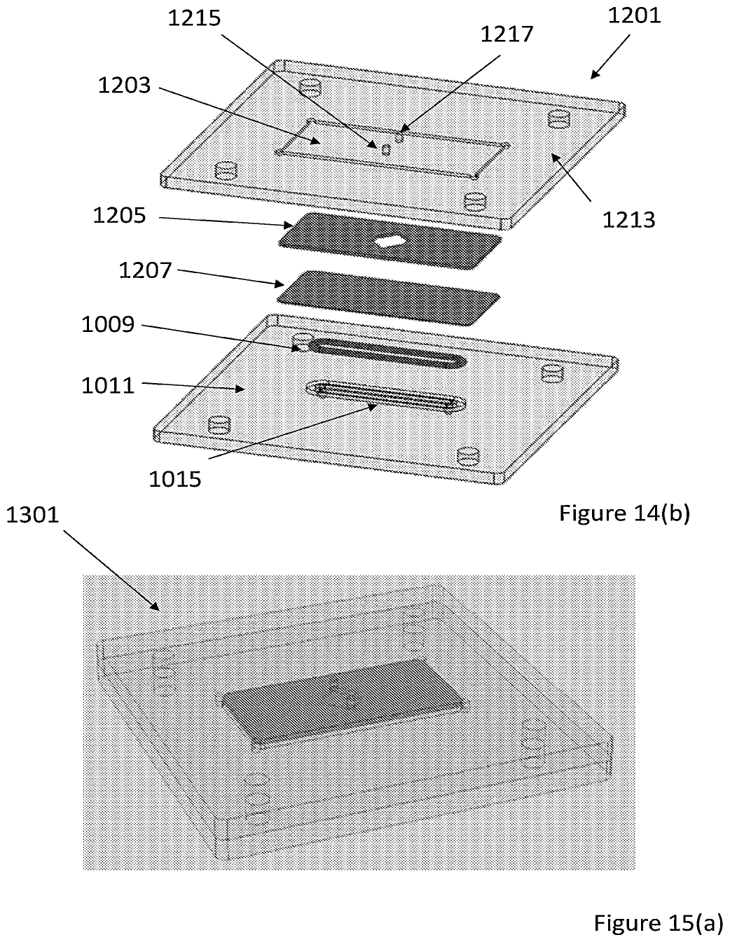

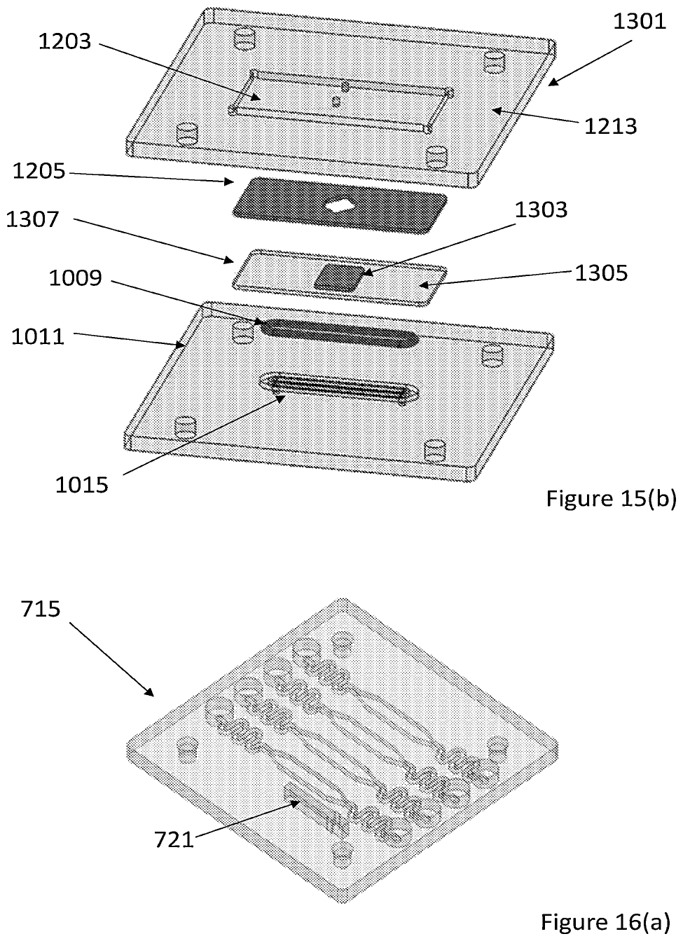

FIGS. 14 and 15 illustrate alternative assemblies 1201, 1301 forming part of microfluidic chips which are also in accordance with the embodiment of FIGS. 2(a) and 2(b) in which a fluidic heat exchanger is employed as the thermally active element for heating the microchannel, but in which the size of the heat transfer sealing layer is varied in order to enable flexible localisation of the temperature control.

FIGS. 14(a) and 14(b) show an assembly 1201 according to an embodiment with full temperature control. The exploded view is given in FIG. 14(b). In this embodiment, the temperature control channel 405 is formed by a support plate 1213 with a seat 1203 for receiving a seal 1205 (e.g. a gasket) having a central aperture, or cavity. In this embodiment, the heat transfer sealing layer 1207 spans the whole of the temperature control channel and covers the microfluidic channel profile 1015 formed in the channel plate 1011. The seal 1205 is arranged to support the heat transfer sealing layer 1207, i.e. the heat transfer sealing layer 1207 is supported by the fluidic heat exchanger, of which only the support plate 1213 and seal 1205 are shown in FIG. 14. As before, an elastomeric seal 1009 (e.g. formed from an O-ring) forms a seal between the heat transfer sealing layer 1207 and the channel profile 1015 to form the microchannel.

In this embodiment, a closed channel is formed by the support plate 1213, the cavity in the seal 1205 and the heat transfer sealing layer 1207 (FIG. 14(b)) after assembling the microfluidic chip 1201. The support plate 1213 comprises two through holes which form an inlet 1215 and an outlet 1217 for fluid heat control of the channel. The inlet 1215 can be connected to hot and cold reservoirs, as appropriate, for input in to the cavity in the seal 1205 for heating and/or cooling of the microchannel.

Thus, in this embodiment the heat transfer sealing layer 1207 covers the entirety of the channel profile 1015 and therefore temperature control of the entire microchannel is possible.

FIGS. 15(a) and 15(b) show an assembly 1301 with a similar arrangement to that of FIGS. 14(a) and (b), and including the same channel plate 1011 but with localized control of the temperature in the microchannel. The assembly 1301 has the same components as the assembly 1201, namely a support plate 1213 with a seat 1203 for receiving a seal 1205 (e.g. a gasket), channel profile 1015 formed in channel plate 1011 and an elastomeric seal 1009 (formed, e.g. from an O-ring) forming a seal between a heat transfer sealing layer 1303 and the channel 1015.

However, in the embodiment of FIGS. 15(a) and (b), the heat transfer sealing layer 1303 only spans a central portion of the channel profile 1015. In this embodiment, the fluidic heat exchanger (of which only a portion is shown) comprises not only support plate 1213 and seal 1205 but also further support plate 1305 which surrounds the heat transfer sealing layer 1303 and thereby supports it in cooperation with the seal 1205.

As the heat transfer sealing layer 1303 is smaller than the channel profile 1015, temperature control is localised to the area intersection between the heat transfer sealing layer 1303 and the channel profile 1015. The further support plate 1305 of the fluidic heat exchanger may comprise an insulating material such as plastic.

As above, in this embodiment, a closed fluidic channel is formed by the backing plate 1213, the cavity in the gasket 1205 and the heat transfer sealing layer 1303 after assembling the assembly 1301.

The backing plate 1213 may comprise non-conductive materials such as plastic (ABS, PMMA, COC, PC, etc.) and manufactured using, for example, laser cutting, milling, plastic injection moulding.

The seal 1205 may be produced from fluorocarbon (such as Viton™), rubber, neoprene, etc. using moulding methods such as extrusion and secured in place with the O-ring groove. Suitable O-rings are commercially available or can be custom-made according to requirements in accordance with methods well known in the art.

The heat transfer sealing layers 1207, 1303 may be formed from, for example copper or aluminium sheets and be fabricated, for example, using wire cut electrical discharge machining (EDM), die cutting or manual cutting with scissors. Alternatively heat transfer sealing layers 1207, 1303 may be formed from, for example, a film, for example a copper or aluminium film.

As discussed above, the reservoirs (not shown) may be a thermostatic electric water or syringe heaters, in which the syringes filled with fluid act as the reservoirs, i.e. baths capable of heating and/or cooling (as required). Suitable water baths are commercially available.

Optionally, a sensor may be incorporated into the microfluidic chip in order to enable feedback control of the temperature control channels.

The assemblies 1201, 1301 further comprise fastening elements to secure the microfluidic chips together (not shown). These are not particularly limited and may include (but are not limited to) a bolt and a nut; a snap lock pin and a spring; and a magnet.

All of the microfluidic chips of the embodiments described above, whether employing thermoelectric pumps (Peltier devices) or fluidic heat exchangers are adaptable and, in all cases, the channel plates could be replaced with a replacement channel plate (optionally along with a replacement heat transfer sealing layer) according to the requirements of the system.

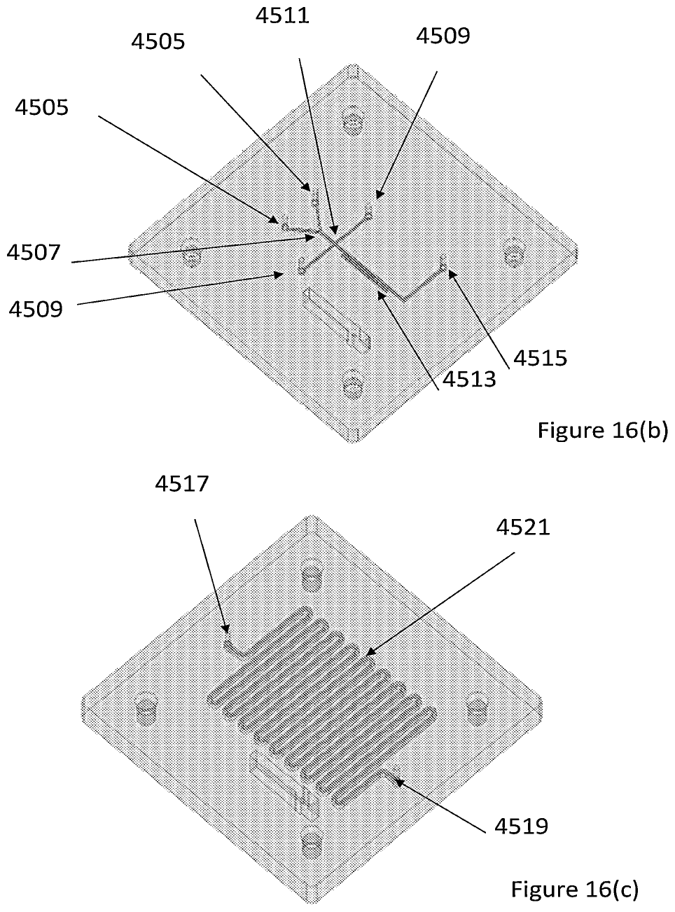

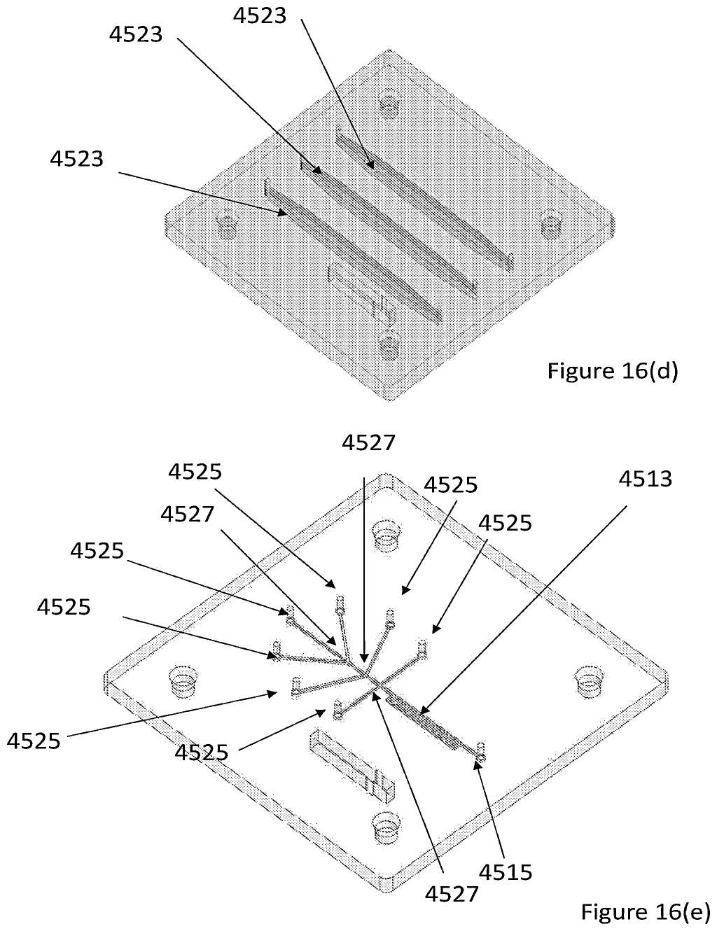

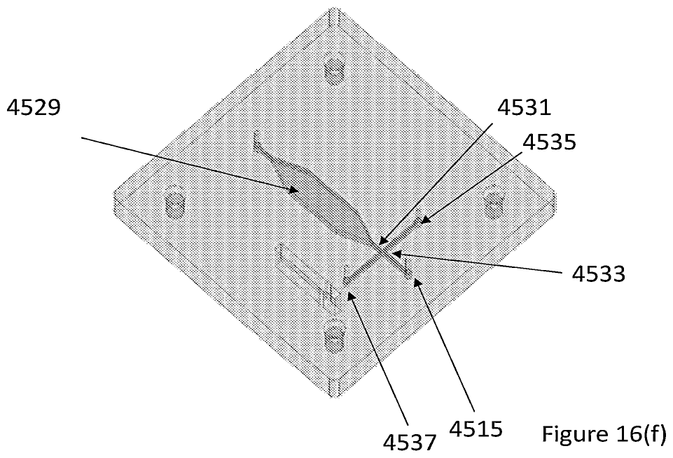

FIGS. 16(a)-(f) show various alternative channel plates for use in any of the microfluidic chips 1, 11, 2, 601, 70, 71, 72, 1001, 1101 according to embodiments described above for different applications, each comprising a least one microfluidic channel profile and an (optional) sensor slot.

FIG. 16(a) shows the channel plate 715 of the microfluidic chips of FIGS. 7 and 8 having four PCR channel profiles as described in detail above in relation to FIG. 7(c). The channel plate 715 further includes sensor slot 721.

FIG. 16(b) shows a channel plate forming a droplet-based micromixer for DNA transformation on chip. The plate includes four inlets connected via two channel junctions in a branch-like arrangement, specifically two inlets 4505 leading to straight channels which meet at a Y-junction 4507 which in turn leads to a channel cross junction 4511 with two further inlets 4509. Cross junction 4511 leads to a serpentine reaction chamber 4513 which in turn leads via an I-shaped section of the channel to outlet 4515. The plate further includes sensor slot 4503.

FIG. 16(c) shows a channel plate suitable for incubation or storage. The channel profile comprises a single input 4517 and a single output 4519 joined by an elongate oscillating (or serpentine) section 4521.

FIG. 16(d) shows a channel suitable for sample loading on the chip with small reagent volumes and includes three, unconnected elongate channels 4523, each of which taper to a point at both ends.

FIG. 16(e) shows a channel forming a droplet-based micromixer suitable for DNA assembly on chip, having seven inlets 4525 connected by linear channels in a branch-like arrangement, three channels joining at each of three sequential junctions 4527 and leading to a serpentine reaction chamber 4513 and outlet 4515.

FIG. 16(f) shows a channel plate suitable for reinjection of a droplet into a channel comprising a relatively broad channel 4529 with tapered ends leading into a narrow channel 4531 which itself joins with two channels from oil inlets 4535 and 4537 at channel junction 4533 leading to outlet 4515.

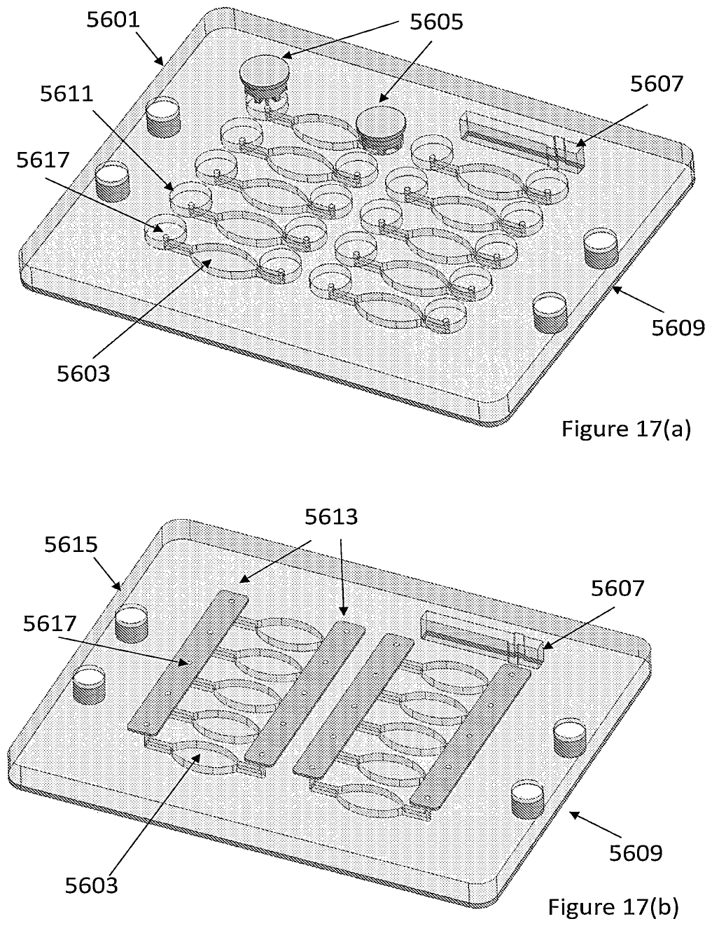

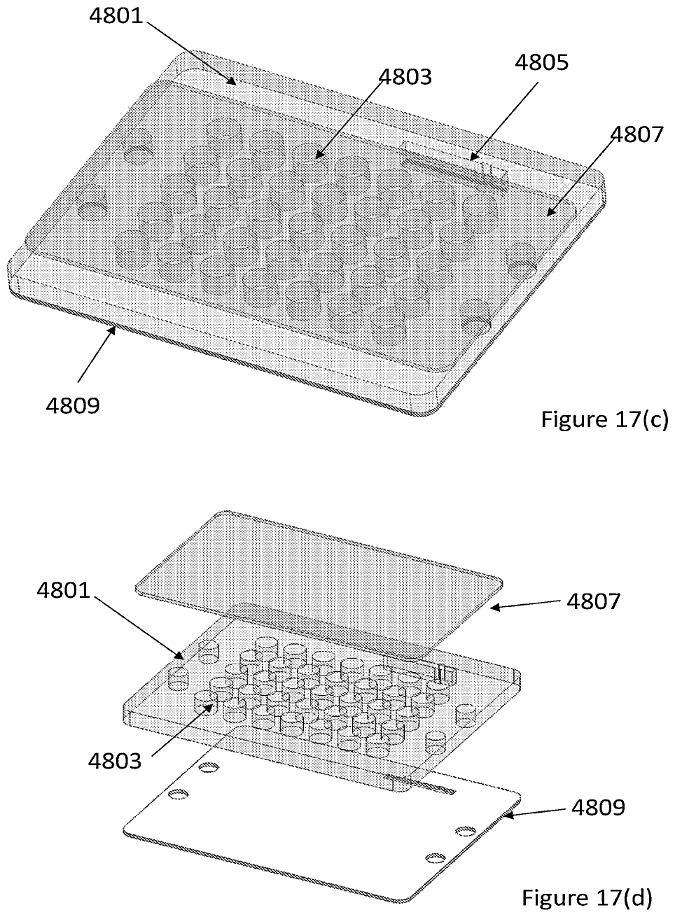

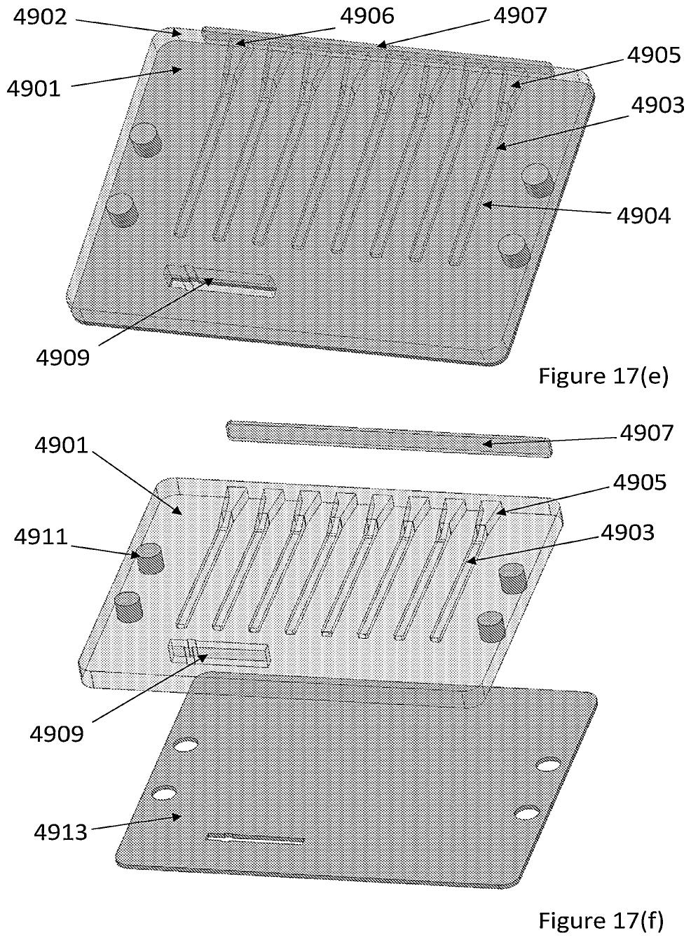

FIGS. 17(a) and 17(b) illustrate further alternative channel plates 5601, 5615, in combination with a heat transfer layer 5609, with channel profiles suitable for, for example, PCR reactions which do not require the oil sandwiching approach, instead employing plugs or an adhesive sealing film to prevent evaporation from the channels. Both channel plates 5601, 5615 include ten channel profiles 5603 with eye-shaped reaction chambers as in the channel profiles of FIG. 9(a) (with the serpentine regions 4001 of the channel profiles of FIG. 9(a) omitted), arranged in two columns. A heat transfer sealing layer 5609 is provided on the underside of the plates 5601 and 5615 and cooperates with each the channel profiles 5603 to form ten microchannels. A sensor slot 5607 is also included in each of the plates 5601, 5615.

In the plug-based alternative shown in FIG. 17(a), each of the channel profiles 5603 includes an inlet and an outlet in the form of small, circular reservoirs 5611 on the upper surface of the channel plate 5601, each operable to receive a circular plug 5605. At the centre of each reservoir 5611 is a small inlet in the form of through-hole 5617 through the channel plate 5601. After the sample is loaded into the microchannels via the inlets, a plug 5605 is inserted into each of the reservoirs 5611 of the channel profile 5603 by interference fitting. The exemplary type of plug 5605 shown in FIG. 17(a) has a central conical structure to plug the small inlet in the centre of the reservoir 5611. A sealed reaction chamber is thus formed by inserting the plugs 5605 into the inlet and outlet of each channel profile 5603 thereby preventing evaporation, the channels 5605 being sealed on the opposite side of the microfluidic relative to that in which the plugs 5605 are inserted by a heat transfer sealing layer 5609, such as a metallic film in accordance with embodiments described above.

The design of FIG. 17(a) additionally enables the chip comprising the plate 5601 to be connected to a flow control system where fluidic connections, such as tubes, can be inserted into a plug with a central through hole. A pipette-free system may use such configuration.

In the adhesive-based alternative shown in FIG. 17(b), the design of the channel profiles 5603 is the same as that of FIG. 17(a) but with the small reservoirs 5611 omitted, i.e. only through-holes 5617 are present at both ends of the channel profiles 5603. Four elongate strips of adhesive sealing film 5613 are arranged on the channel plate 5615 over the through-holes 5617, two strips for each row of channel profiles 5603. These strips of adhesive sealing film 5613 prevent evaporation of a sample contained within the channel. The strips of adhesive sealing film 5613 form sealed reaction chambers in cooperation with the heat transfer sealing layer 5609 and the channel profiles 5603.

The designs of FIGS. 17(a) and 17(b) are more compact than those shown in, for example, FIG. 9(a)-(e) because they do not require serpentine regions 4001 to prevent evaporation and therefore more channels 5603 can be fitted onto a single chip 5601, 5615.

Although FIGS. 17(a) and 17(b) show channel plates suitable for use in PCR reactions, the methods of sealing channels to prevent evaporation shown in those figures could be used in conjunction with any channel design disclosed herein.

FIG. 17(c) illustrates a further alternative channel plate 4801, in combination with a heat transfer sealing layer 4809, in which a plurality of through holes 4803 (in this case 35) are formed. The heat transfer sealing layer 4809 is arranged on one face of the channel plate 4801 and a sealing film 4807 is arranged on the other side of the channel plate 4801. An exploded view of the assembly is shown in FIG. 17(d). The chip 4801 also comprises a sensor slot 4805. When assembled, all of the through holes 4803 are simultaneously sealed by the heat transfer sealing layer 4809 and sealing film 4807 to form micro-reservoirs, or equivalently micro-wells. The arrangement of channel plate of 4801 of FIGS. 17(c) and (d) enables the number of reaction chambers on a chip to be significantly increased relative to other designs. The sealing film 4807 further prevents evaporation of the samples from the through holes 4803.

FIG. 17(e) and FIG. 17(f) illustrate a yet further alternative channel plate 4901, in combination with heat transfer sealing layer 4913, in which a plurality of microchannel profiles 4903 (in this example eight) having a microcuvette profile are formed. FIG. 17(e) shows a perspective view of the assembly and FIG. 17(f) shows an exploded view. Each microchannel profile 4903 comprises a straight elongate portion 4904 and a tapering portion 4905 which opens out towards an inlet 4906 in the form of an aperture on one edge 4902 of the channel plate 4901, such that the inlet 4906 is wider than the straight elongate portion 4904 of the channel. A sealing film 4907 is positioned along the edge 4902 simultaneously sealing each inlet 4906 of each of the microchannel profiles 4903. The heat transfer sealing layer 4913 is arranged on one face of the channel plate 4901, thereby cooperating with the microchannel profiles 4903 to form eight microcuvettes. The channel plate 4901 further comprises a sensor slot 4909. The assembly is held together by magnets 4911, as described above in accordance with embodiments.

In use, each microfluidic reservoir may be pre-loaded with reagents required for an amplification reaction either in liquid form or lyophilized form. Then, a user may add samples (such as nasal swab samples) and, optionally, water (in the case of, for example, pre-loaded lyophilized reagents) directly into the microfluidic reservoirs via the inlet 4906 by using a micro/nano-dropper or micropipette, and seal the inlet 4906 with the sealing film 4907. This approach enables minimal sample preparation steps. Alternatively, a pump, such as a portable pressure pump, may be used to drive samples into each reservoir by pressurized air—this further minimizes the contact of the user with the samples.

In one example protocol employing the assembly according to FIGS. 17(e) and 17(f), 5 μL of a nasal swab sample is loaded into a microfluidic reservoir pre-loaded with 20 μL of direct RT-PCR reagents in liquid form (examples include Resolute 2.0 SARS-CoV-2 Direct Detection Kit, Focusgen Influenza Virus and SARS-CoV-2 Multiplex Direct RT-PCR Test Kit).

In another example protocol, 5 μL of a nasal swab sample and 15 μL of water are loaded into a microfluidic reservoir pre-loaded with lyophilized direct RT-PCR reagents (examples include Xfree™ COVID-19 Direct RT-PCR).

As in the embodiments discussed above, all of these plates could be mass produced by injection moulding, hot embossing, laser cutting (e.g. with a CO₂ laser) 3-D printing, plastic milling in accordance with methods known in the art.

For mass production, plastic injection moulding may particularly be employed.

For low volume production, machine methods such as milling or laser cutting may particularly be employed. Advantageously, laser cutting provides a particularly fast way of producing the channel profiles. Advantageously, milling achieves high accuracy and a good surface finish.

Thus, the channel profile could be easily changed to suit various applications. As shown above, the possible channel profiles include but are not limited to that of a micromixer or a drop-based micromixer, a straight channel, a spiral channel, etc. for different purposes, including but not limited to polymerase chain reaction; DNA transformation, and DNA assembly; Eukaryotic cellular studies requiring thermal treatment e.g. study of heat shock responses in mammalian cells; heat treatment of proteins for biophysical studies, e.g. denaturation; isothermal DNA amplification, such as LAMP and RT-LAMP, recombinase polymerase amplification (RPA), nucleic acid sequence-based amplification (NASBA), strand displacement amplification (SDA), and helicase dependent amplification (HAD).

In particular, one or more microfluidic chips 1, 11, 2, 601, 70, 71, 72, 1001, 1101 in accordance with an embodiment described above, in particular those of FIGS. 7-9, may be employed in Polymerase Chain Reaction (PCR), such as qPCR and RT q-PCR, isothermal DNA amplification, such as LAMP and RT-LAMP, and recombinase polymerase amplification (RPA) for the detection of nucleic acids (DNA or RNA). Examples of such functionality are discussed below. The nucleic acids may be from a microorganism, virus, human, animal, plant, or of a synthetic source. One or more of the microfluidic chips according to embodiments may also be employed in direct RT-PCR and direct RT-LAMP, where no prior extraction is required, using a single microfluidic chip according to an embodiment described above with temperature control.

The temperature control microfluidic chips according to any of the above described embodiments can function as standalone units and may enable samples to be loaded directly into its channels. In other embodiments the microfluidic chips according to the embodiments described above may be connected to other functional modules for various temperature-dependent biological processes including isothermal and/or cyclic thermal control (polymerase chain reaction, isothermal amplification of nucleic acid, DNA assembly, DNA transformation, freeze thaw on chip, etc.); rapid temperature change in a microchannel or a shallow channel (biofilm growth, monolayer cell culture), etc. Multiple functional modules can be connected to form a multi-step nucleic acid quick extraction and amplification and coupled with a detection unit for diagnosing infectious disease such as COVID-19, influenza, etc. In other embodiments, multiple functional modules may be connected to the microfluidic chips in accordance with embodiments to form a multi-step multi-function biomanufacturing line (for example, a droplet generation and merging/mixing module).

For example, one or more microfluidic chips 70, 71 according to an embodiment described above in relation to FIGS. 7(a)-(e) may be employed in order to perform amplification of nucleic acid.

In another example, at least one microfluidic chip 601 according to an embodiment described above in relation to FIG. 6(a)-(d) may be employed in order to perform DNA assembly or transformation. These processes may require both isothermal and cyclic temperature control.

In embodiments, the microfluidic chip 601 having a main channel profile in the form of a either mixer (FIG. 6(d)), or a drop-based mixer (FIG. 6(c)) may be employed to perform DNA assembly and/or DNA transformation with temperature control on chip. In this case, the microfluidic chip 601 can function as a standalone chip for a simple biological process. In the case that a sample, or reagent volume is limited, this microfluidic chip 601 could be connected with a droplet module for forming a multi-step and multi-function biomanufacturing line. The droplets can also be prepared off-chip and be loaded onto the microfluidic chip 601 for temperature control. The use of the microfluidic chip 601 in this way is discussed in the below according to embodiments.

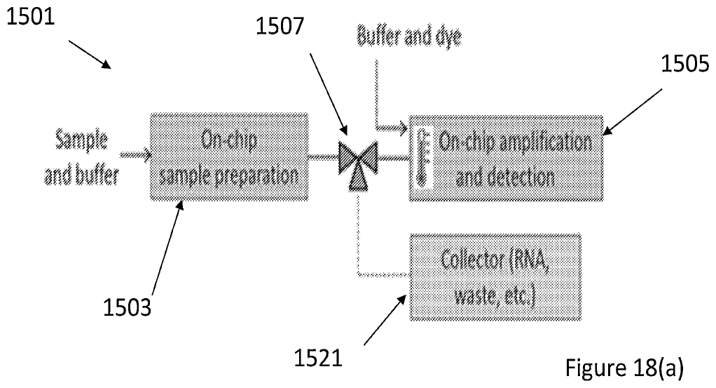

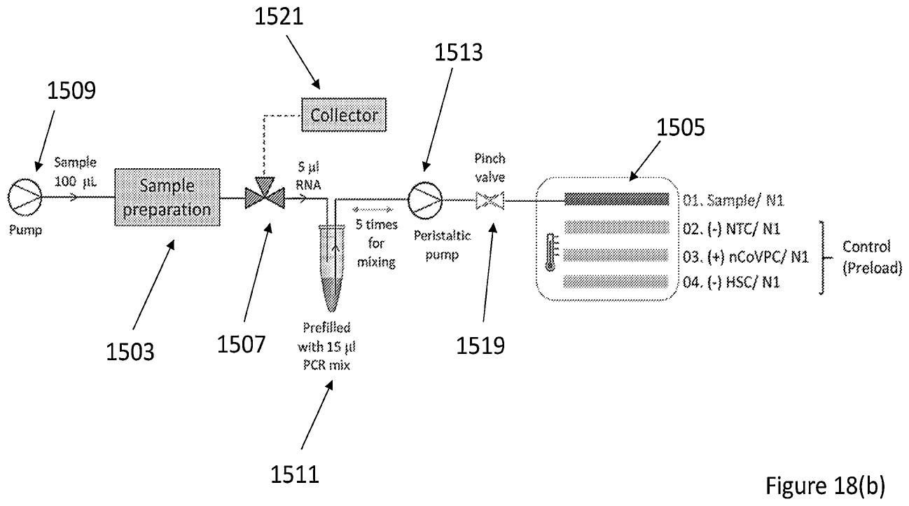

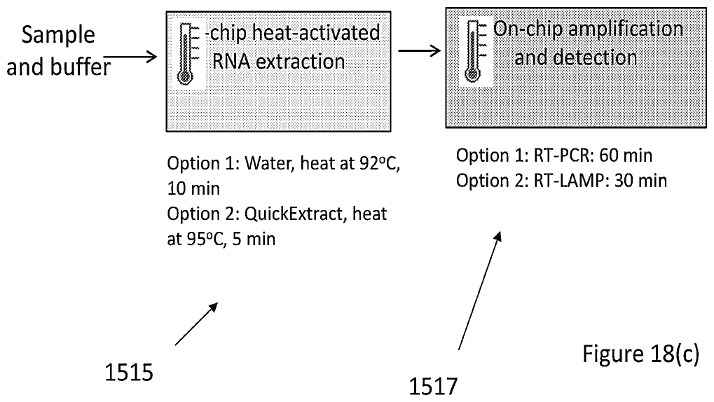

In an embodiment, one or more microfluidic chips in accordance with an embodiment described above are connected to other functional modules. Example systems according to these embodiments are shown in FIGS. 18(a)-(c). In some embodiments, all of the modules (i.e. microfluidic chips in the platform) may have temperature control in accordance with embodiments described above. In other embodiments, only some of the modules may have temperature control functionality in accordance with embodiments described above. This will vary according to the requirements of the step to be performed on a particular module and can be adapted to suit the requirements of the protocol. In some embodiments, only one module may require temperature control. These examples will be discussed below.

FIGS. 18(a)-(c) show a number of arrangements of microfluidic chips as platforms for nucleic acid detection of infectious disease.

FIG. 18(a) shows a microfluidic platform 1501 comprising two microfluidic modules: a sample preparation module 1503 where on-chip sample preparation is carried out and module 1505 wherein the on-chip amplification and detection is carried out. The first sample preparation module 1503 does not require temperature control and may be a conventional microfluidic chip without temperature control functionality, or it may be a microfluidic chip in accordance with embodiments. The second module 1505 comprises a microfluidic chip according to an embodiment described above with temperature control functionality, in particular according to the embodiments of FIGS. 7(a)-(e). The platform further comprises a micro-valve 1507 connecting the two microfluidic chips and a collector module 1521.

FIG. 18(b) shows the platform of FIG. 18(a) in more detail including an example of flow control according to an embodiment. In this embodiment, the sample is pumped into the sample preparation chip 1503 with pump 1509. The two modules 1503, 1505 are linked through a micro-valve 1507 enabling independent fluid flow control. The prepared samples are mixed with PCR mix 1511 using peristaltic pump 1513 before being directed to the first channel in temperature control module 1505 via pinch valve 1519.

The module 1505 includes a microfluidic chip in accordance with embodiments having temperature control including a channel plate which contains four PCR channels, each of which can be used as an independent reaction chamber. For example, channel 01 can be used to test the N1 gene of SARS-CoV-2, while channels 02, 03, and 04 can be used to test required controls, particularly a no template control, a 2019-nCoV positive control and a human sample/specimen control respectively.

FIG. 18(c) shows another example of a system for a platform for nucleic acid amplification and detection according to an embodiment. In this embodiment, heat-activated RNA extraction is performed on a first module, 1515, followed by on-chip amplification and detection on the second module 1517. Both modules 1515, 1517 include microfluidic chips having temperature control capability according to any of the embodiments described above.

A number of protocols are known in the literature for processes suitable for use with the platform of FIG. 18(c). For example, in accordance with one protocol (Jürgen Durner, Siegfried Burggraf, Ludwig Czibere, Tobias Fleige, Arleta Madejska, David C Watts, Frank Krieg-Schneider, and Marc Beckera, Fast and simple high throughput testing of COVID-19, Dent Mater. 2020 Apr. 6), the platform may take a patient's nasal swab and mix with DNAase/RNAase free water with the volume ratio of 1:1 and heat at 92° C. for 10 minutes in module 1515. The effluent may then be transferred and mixed with the one step reverse transcription PCR reagent or a RT-LAMP reagent in module 1517 and the temperature controlled according to the appropriate kit protocol. For example, for RT-PCR using the commercially available Takara One Step PrimeScript™ RT-PCR kit (Takara Bio Inc.), the temperature of the chip may be controlled so as to heat the sample up to 42° C., holding for 5 minutes, 95° C. and holding for 10 s, followed by 40 cycles of 95° C. for 5 s and 60° C. for 20 s, and finally a cool down to 4° C., as shown in FIG. 34.

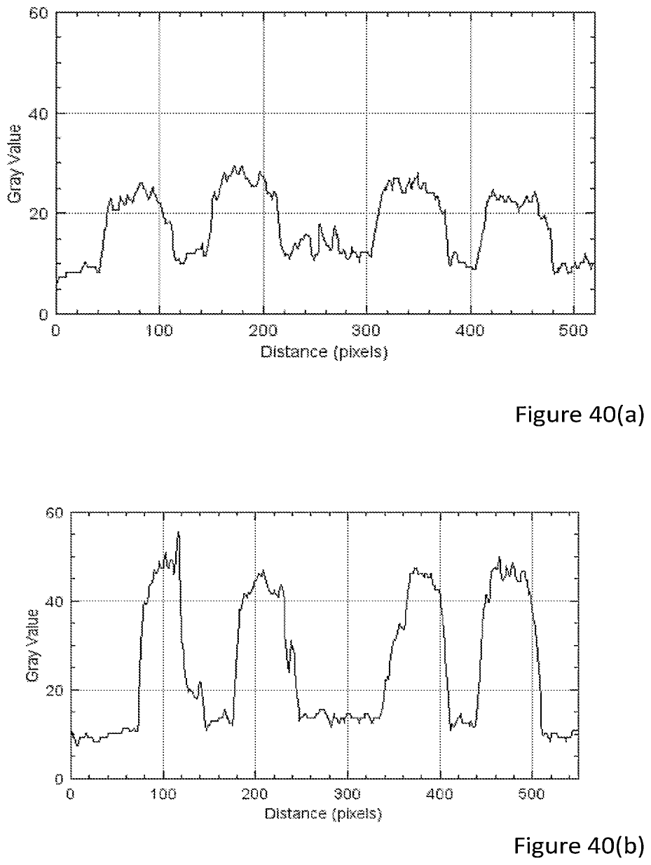

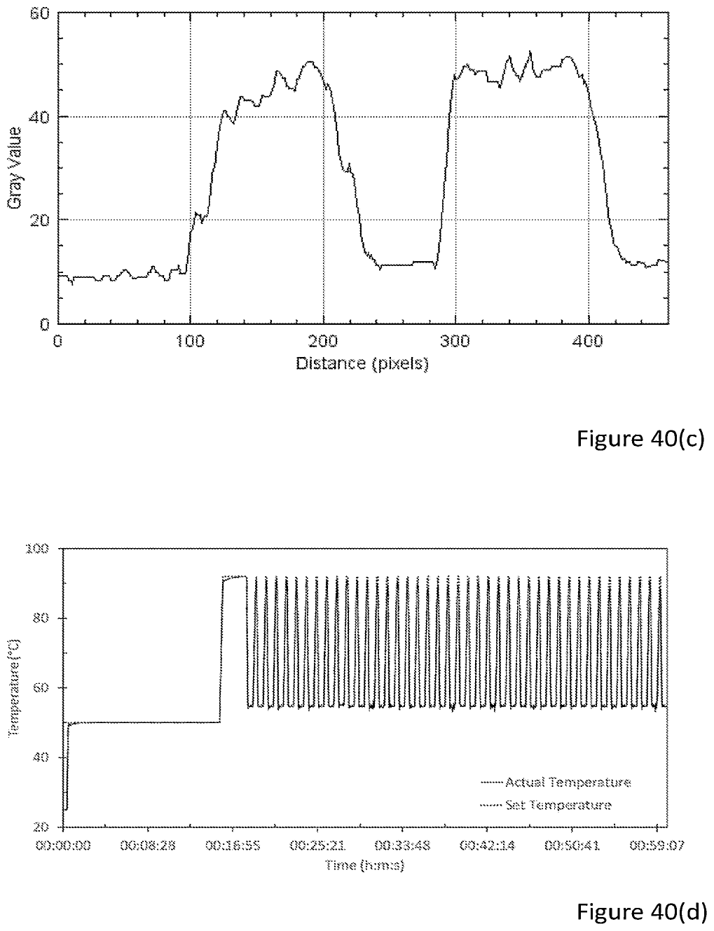

Other commercially available examples of one step reverse transcription PCR reagents include TaqPath™ 1-Step RT-qPCR Master Mix (ThermoFisher Scientific) for which the corresponding protocol comprises heating the sample up to 50° C., holding for 15 minutes, 95° C. and holding for 2 minutes, followed by 42 cycles of 95° C. for 3 s and 55° C. for 30 s, as shown in FIG. 40(d).

In accordance with another protocol (Viet Loan Dao Thi, Konrad Herbst, Kathleen Boerner, Matthias Meurer, Lukas P M Kremer, Daniel Kirrmaier, Andrew Freistaedter, Dimitrios Papagiannidis, Carla Galmozzi, Megan L. Stanifer, Steeve Boulant, Steffen Klein, Petr Chlanda, Dina Khalid, Isabel Barreto Miranda, Paul Schnitzler, Hans-Georg Kräusslich, Michael Knop, Simon Anders, A colorimetric RT-LAMP assay and LAMP-sequencing for detecting SARS-CoV-2 RNA in clinical samples, SCIENCE TRANSLATIONAL MEDICINE, 2020, Aug. 12), the patient's nasal swab may be heated at 95° C. for 5 minutes in module 1515. The effluent may then be transferred and mixed with a LAMP reagent in module 1517, for example WarmStart™ Colorimetric LAMP 2× Master Mix (DNA & RNA) from New England Biolabs. The temperature of the chip may be controlled to heat the sample to 65° C. and hold for 30 minutes. Positive samples may be determined by the visible colour change from pink to yellow of the reagent at the end of the incubation at 65° C.

In another protocol, the platform may take a patient's nasal swab and mix with QuickExtract Extraction solution (Lucigen) with a volume ratio of 1:1, followed by a short heating step at 95° C. for 5 minutes (Alim Ladha, Julia Joung, Omar O. Abudayyeh, Jonathan S. Gootenberg, Feng Zhang, A 5-min RNA preparation method for COVID-19 detection with RT-qPCR, 2020) on the first module 1515. As above, the effluent will then be transferred and mixed with the one step reverse transcription PCR reagent in module 1517 for corresponding temperature control as discussed above.

According to these protocols, the targeted time from sample to result may be 35 minutes, i.e. 5 minutes for quick heat-activated RNA extraction and 30 minutes for isothermal amplification RT-LAMP and detection. Longer times may also be employed such as 65 minutes, comprising, for example 5 to 10 minutes for quick heat-activated RNA extraction and 60 minutes for one step reverse transcription PCR and detection.

With the on-chip temperature control using a microfluidic chip according to an embodiment described above, RT-PCR or RT-LAMP can be performed at the desired temperature profiles and the signal detected either by colour change or by a fluorescence signal. The detection of such signals will be discussed in detail below. This platform is not only applicable to SARS-Cov-2 detection but also for other infectious diseases through similar protocols. Furthermore, the modularity and on-chip temperature control enable the detection pipelines to be composed of various combinations of sample preparation modules and detection methods.

Thus, through this lab-on-chip system, sample to results can be achieved in accordance with literature protocols in 35 to 65 minutes. In addition, the samples fully reside within closed fluidic circuits inside the microfluidic platform. No manual pipette of sample in and out is required which reduces the risk of cross-contamination. Furthermore, there is no need for centralized laboratory facilities such as a centrifuge, plate reader, or gel imager etc. This makes it possible to be readily deployed in airports, field clinics, community clinics, etc.

As discussed above, although the embodiments of FIGS. 18(a)-(c) show on-chip temperature control for RT-PCR or RT-LAMP requiring RNA extraction as a first step, microfluidic chips according to embodiments described above may also be employed in direct RT-PCR or direct RT-LAMP, where no prior extraction is required. In these embodiments, only a single PCR microfluidic chip with temperature control such as module 1505 is required.

According to one such protocol (Soon Keong Wee, Suppiah Paramalingam Sivalingam, and Eric Peng Huat Yap, Rapid Direct Nucleic Acid Amplification Test Without RNA Extraction for SARS-CoV-2 Using a Portable PCR Thermocycler, 2020), the platform may take a patient's nasal exudate or sputum and mix with Sputasol (Oxoid, Genes 2020, 11, 664 3 of 13 Basingstoke, England) in a volume ratio of 1:1. The effluent may then be mixed with primers and a onestep reverse transcription PCR reagent (for example, VitaNavi Direct One-Step S/P RT-qPCR TaqProbe Kit) to a total reaction volume of 20 μl and loaded onto module 1505 for corresponding temperature control and detection. The temperature conditions may comprise reverse transcription at 50° C. for 15 min and initial denaturation at 95° C. for 1 min, 45 cycles of denaturation at 95° C. for 10 s and annealing at 55° C. for 45 s. This direct PCR assay could be completed in 72 minutes. To further reduce turnaround time and reagent cost, the protocol mentioned above could be further modified to be performed using a total reaction volume of 10 μl, with temperature conditions accordingly modified, such as a modified reverse transcription at 50° C. for 5 min and initial denaturation at 95° C. for 30 s, 40 cycles of denaturation at 95° C. for 10 s and annealing at 55° C. for 15 s. This fast, direct PCR assay could thus be completed in 36 min.

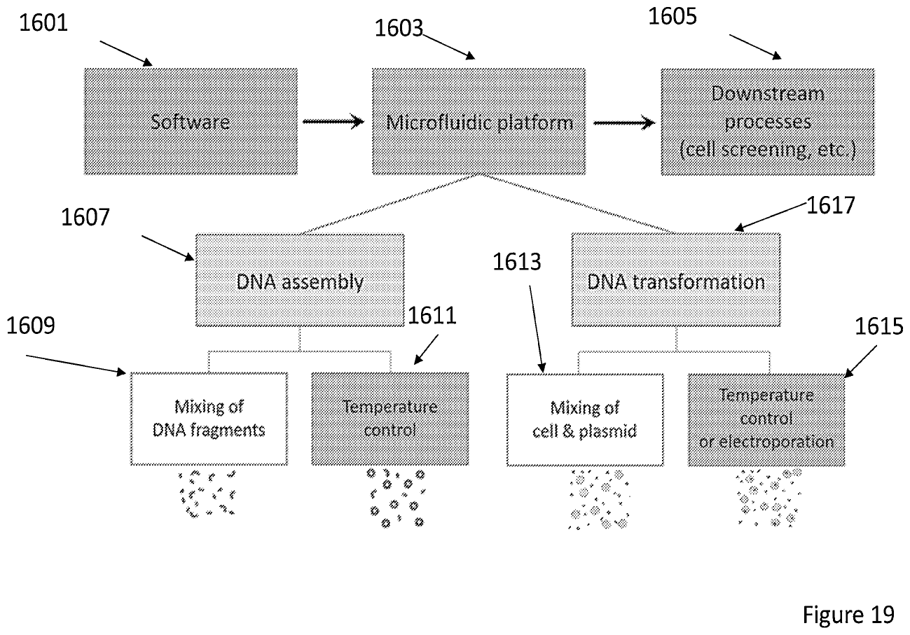

A microfluidic chip according to an embodiment described above may also form a basic unit to form a composable multi-step and multi-function biomanufacturing line. The concept of a modular biomanufacturing line or platform is illustrated in FIG. 19. A biomanufacturing line comprises a microfluidic platform 1603 composed of multiple units of modules, each of which performs singular or a subset of functions in a complex multi-step biological process. In an embodiment, one or more of the individual modules 1611, 1615 includes a microfluidic chip according to an embodiment described above comprising temperature control. The microfluidic platform itself may reside in the overall process between software 1601 and downstream processes 1605 such as cell screening etc.

FIG. 19 specifically shows the example of a biomanufacturing line for DNA assembly 1607 and DNA transformation 1617 employing microfluidic chips according to embodiments described above. Each of these steps themselves comprises two sub steps, firstly the mixing of DNA fragments 1609 or cells and plasmid 1613, as appropriate, and secondly a temperature control step 1611, 1615 which, in an embodiment, is performed on a microfluidic chip according to an embodiment discussed above.

The whole platform may include, but is not limited to, a droplet module encapsulating DNA fragments for DNA assembly, a droplet module for encapsulation and merging/pico-injection of plasmid to bacteria for DNA transformation and temperature control modules (for incubation) according to embodiments.

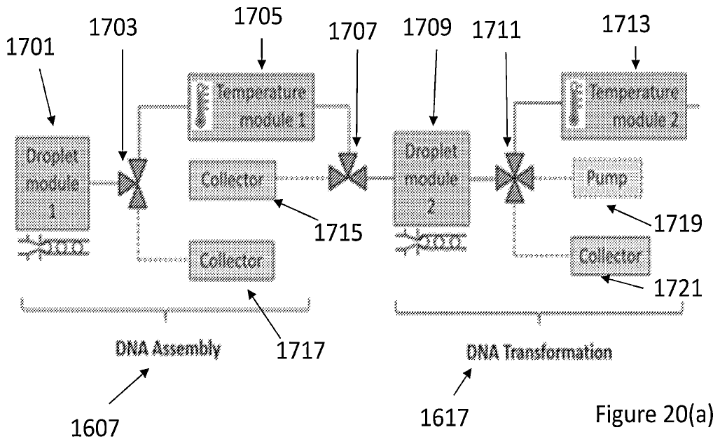

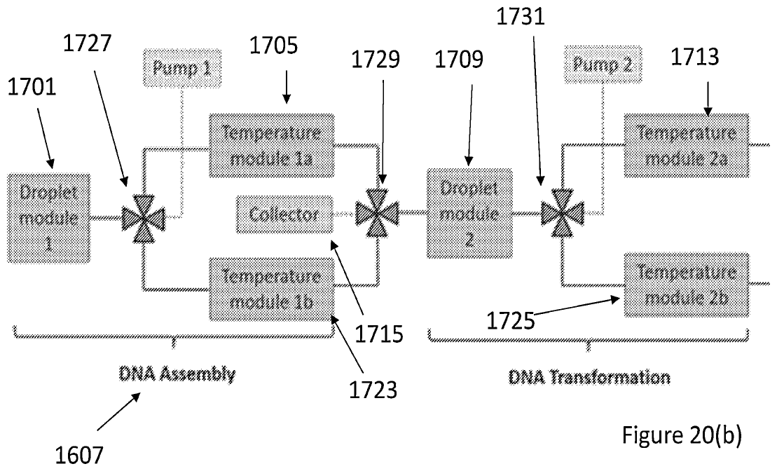

FIG. 20(a) illustrates a detailed configuration of the biomanufacturing line of FIG. 19 according to an embodiment. Combinations of DNA fragments are first encapsulated in droplets in the droplet module 1701 which is a microfluidic channel. The droplets are then infused via microvalve 1703 into a temperature control module 1705 comprising a microfluidic chip according to an embodiment described above for the DNA assembly to take place. Upon completion of DNA assembly, the assembled plasmids in droplet are then infused via microvalve 1707 into another droplet module 1709 where bacteria are injected or merged into the plasmid droplets. The merged droplets are finally moved into another temperature module 1713 comprising a microfluidic chip according to an embodiment described above via valve 1711 using pump 1719, to perform DNA transformation. The obtained product can be funnelled and collected (in collector 1721) or funnelled into more functional modules (such as colony picking). The basic functional modules are fluidically isolated through off-chip or on-chip micro-valves and/or manifolds such as 1703, 1707 and 1711 and samples can be collected via collector 1717, 1715 and 1721.

Thus, in this embodiment four chip modules 1701, 1705, 1709, 1713 are connected to form a biomanufacturing line for DNA assembly and DNA transformation. In embodiments, collectors 1717, 1715 and 1721 can be used for sample collection, waste disposal, pressure relief, etc. In an embodiment, the valves may be electrically controlled to enable automation of the platform.

In addition to serial connection of the functional microfluidic modules, microfluidic chips according to embodiments described above also enable scaling of functional modules by connecting them modules in parallel and controlling them with off-chip/on-chip micro-valves and/or manifolds. This is important in scaling the biomanufacturing line when some processes have long reaction times. FIG. 20(b) illustrates an embodiment in which one additional temperature module comprising a microfluidic chip according to an embodiment described above is added per stage to double the throughput relative to that of the embodiment of FIG. 20(a), i.e. the platform is a six-chip platform. In this platform, the same components are present as in FIG. 20(a) but temperature module 1b 1723 is added to the line through a 4-port micro-valve 1727. Droplets can be channelled into Temperature module 1b 1723 when Temperature module 1a 1705 is at full capacity and incubating for DNA assembly reactions. Similarly, Temperature module 2b 1725 is added to receive droplets when Temperature module 2a 1713 is at full capacity and incubating for the DNA transformation process. The configuration is not limited to adding one additional module per stage but can be scaled out to a large number of modules with using automation such as electronic valve control.

Thus, in this embodiment, more temperature modules comprising a microfluidic chip according to an embodiment described above may be added to improve the system throughput when temperature control over a long duration becomes a bottleneck in the process. As before, the collector 1715 could be used for sample collection, waste disposal, pressure relief, etc. In embodiments, the valves 1727, 1729 and 1731 may electrically controlled to enable automation of the platform.

In an embodiment, a microfluidic chip according to an embodiment described above with temperature control above may be employed as part of a DNA transformation module. In an embodiment, the microfluidic chip used for this process may have a channel plate design according to the embodiment of FIG. 16(b).

In this embodiment, DNA plasmids are mixed with bacterial competent cells, followed by encapsulation into a droplet. In this process, the plasmid with an antibiotic resistance gene and target gene will be transformed into the host bacterial competent cells under the appropriate temperature conditions. DNA transformation protocols for tube transformations such as that published by Zymo Research with the ratio of DNA plasmids to bacterial competent cells adjusted to account for the microscale of microfluidic channels may be employed. In an embodiment this process is performed on a microfluidic chip according to an embodiment described above with temperature control. The droplets containing transformed samples may be collected directly onto agar plates followed by incubation at 37° C. for the colonies to grow in order to validate the transformation efficiency. The transformed bacteria will grow and express desired protein for gene cloning or protein synthesis.

Components for assembling the microfluidic platforms discussed above such as microvalves (including electronic microvalves), pumps are commercially available.

Detection Module

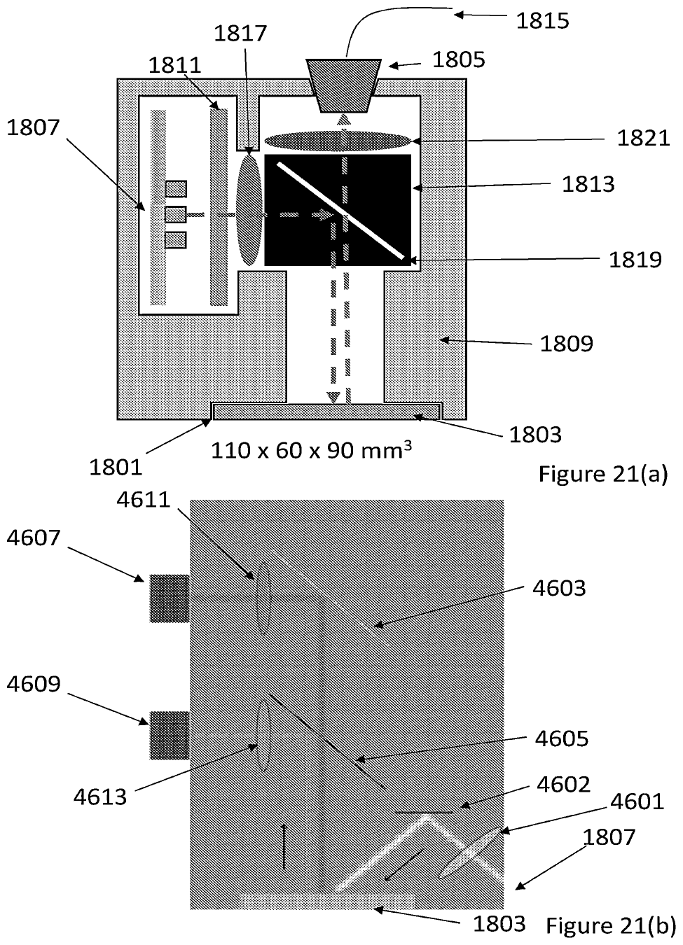

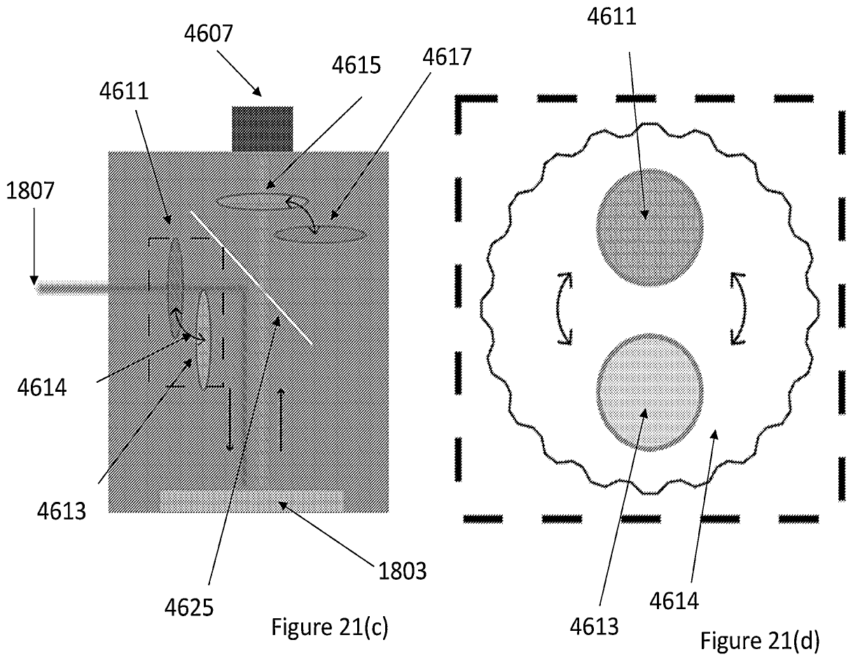

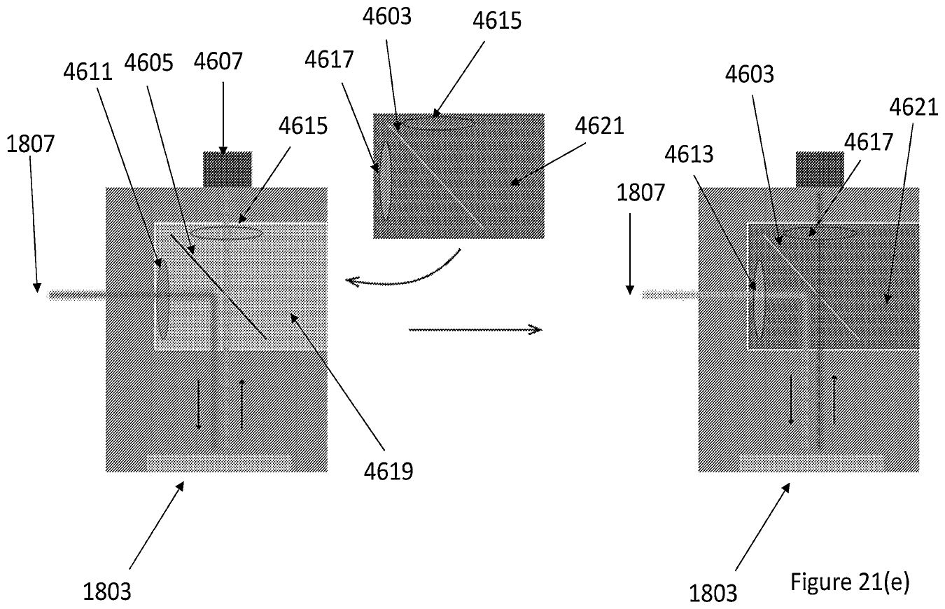

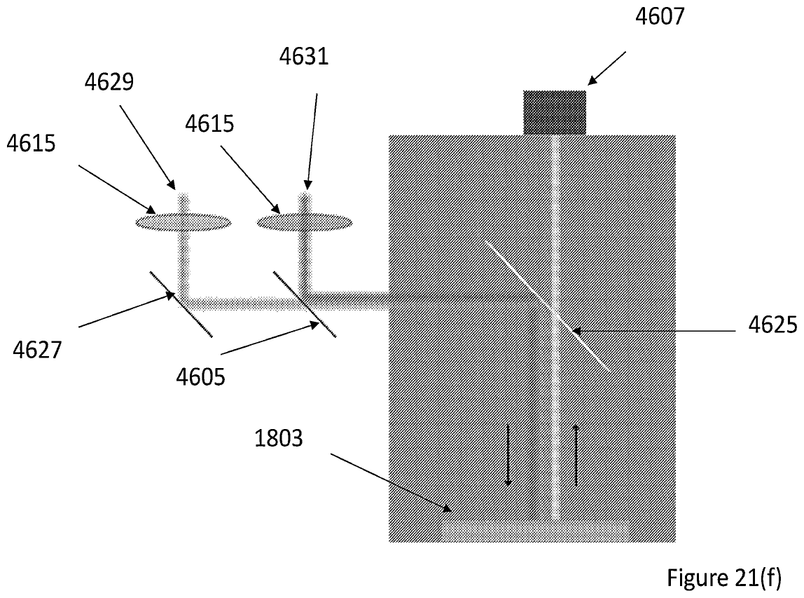

FIGS. 21(a)-(f) and 22 show detection modules, equivalently detectors, according to embodiments for detecting light (visible or otherwise) reflected from or emitted from a plurality of samples.

In the embodiments described below, the samples are contained within one or more microfluidic chips, including (but not limited to) one or more microfluidic chips according to any one of the embodiments described above. However, is envisaged that detectors according to embodiments described below may be applied to processes in which the samples are contained within a paper-based platform, lateral flow strips or sample holders such as a 4 well plate (e.g. L×W of 66 mm×66 mm) with or without temperature control, such as in fluorescence immunoassay applications (antigen-based detection).

Likewise, although the below description is primarily directed to embodiments in which the detector is employed with a microfluidic device having four or more microfluidic channels, the person skilled in the art will appreciate that detectors according to embodiments may also be employed with microfluidic devices having greater or fewer microfluidic channels.

In general, detectors according to embodiments comprise a slot 1801 for receiving a microfluidic chip 1803, a detector 1805 in the form of a camera and illumination source (or a plurality of illumination sources) 1807 for illuminating the sample.

In the embodiment of FIG. 21(a), the detection unit is composed of an ambient light shielding cassette 1809 housing optical components. In embodiments, the optical components may include but not be limited to one or more optical diffusers or collimators (e.g. a condenser lens) 1811, an illumination source 1807, a filter cube 1813 and a detector 1805 such as a camera, photodiode or PMT. It is envisaged that the optical components of the detector may be selected according to the precise configuration of the optical set up and sample from which data is to be collected. In an embodiment, a microprocessor in the detector (not shown) is used to transmit via connection 1815 the detected signal to a local device such as a laptop or a smartphone containing an analysis toolbox for analysis data received at the detector.

In an embodiment, the detector comprises a camera arrangement having a large field of view. In an embodiment, the detector may be configured such that the camera receives signals from all the reaction channels on the channel plate simultaneously, i.e. without mechanical movement of light source and/or detection system. In other words, the field of view of the camera is sufficiently large as to capture signals from all of the channels simultaneously. For example, in the case that the detector is employed with a microfluidic chip 70,71 according to the embodiment of FIG. 7(a) or (b), the arrangement of the camera and microfluidic chip 70, 71 may be such that the camera receives signals from all four microfluidic channels 701 on the microfluidic chip 70, 71 without requirement movement of any component. In an embodiment, this may be achieved using low magnification of the samples.

In an embodiment, the camera has a field of view (FoV) larger than the microfluidic chip 1803 loading the sample.

In an embodiment the FoV may be greater than 2 mm×2 mm, particularly greater than 20 mm×20 mm, more particularly greater than 40 mm×40 mm. The FoV may take any shape (e.g. rectangular, circular, irregular).

Advantageously, having a FoV larger than the microfluidic chip 1803 loading the sample enable the detection module to perceive the sample in 2D, i.e. shapes, number of samples, etc. The 2D FoV allows the detection module to capture signals from multiple samples and thus improves the throughput. The larger the FoV, the greater the potential throughput of samples. For example, for multi-channel microfluidic chip-based PCR as discussed above, all four (or six) channels may be accommodated and captured simultaneously. Additionally, systems according to embodiments may be advantageous in digital PCR applications which fluorescent signals from thousands of micro-droplets need to be simultaneously assessed.

In particular, the detector may employ a white light, single wavelength or multiple wavelength LED array or high-power LED with or without a fibre optic cable and a diffuser in order to illuminate all of the samples in the FoV uniformly.

Because detectors according to this embodiment advantageously do not require capability to move the detector and/or the light, the systems according to embodiments are compact and cost effective. The added advantage of larger FoV and lack of any moving parts includes shorter processing time as more samples can be processed simultaneously.

In an embodiment, the detector comprises only a single lens comprised within the camera (there is no lens in the optical path between the light source and the camera), thus rendering it more compact and with larger field of view and more cost effective compared to using an objective lens set. Further, detectors according to this embodiment have low power consumption. They are light, portable, can be battery operated and can be easily deployed at any location.

Thus, the detection module according to embodiments is designed to measure light intensity and not to capture a sharp image, therefore it does not require the use of additional lenses to prevent chromatic aberration. It is akin to using the camera as an array of micro-photodiodes. As such, the system can function without a focusing lens and is therefore lighter, more compact and more cost-effective than detectors employing objective lens sets.

In an embodiment, the distance between the LED and the excitation filter is approximately 10 mm, the filter cube and the microfluidic chip is approximately 26 mm, and the camera and the emission filter is approximately 5 mm. Suitable LEDs for use in this embodiment are Osram GV QSSPA1.13 LEDs. A suitable filter cube for use in this embodiment is Olympus U-MWB2. A suitable camera for use in this embodiment is Arducam OV5642.

In an embodiment, the camera employed in detectors according to embodiments may comprise an array of sensors. In an embodiment, the camera may comprise an array of photodiodes. This advantageously enables spatial resolution to be retained when capturing such a large field of view. This gives the detector the ability to analyse signals which are sensitive to spatial domain such as particle/droplet formation. This is because an image is taken by the detection module array instead of a single point value. In an embodiment, the resolution of the sensor array is greater than about 15 μm at plane of the microfluidic chip, particularly greater than about 10 μm.

In an embodiment, the detection modules according to embodiments can operate in two modes: fluorescence mode and colourimetry mode.

In the embodiment of FIG. 21(a) the detector is operating in fluorescence mode. In fluorescence mode there may be two approaches to optical detection setup according to embodiments: with a filter cube or with a pair of polarisers. In the embodiment of FIG. 21(a), the detection module consists of a light shielding cassette 1809, an illumination source 1807 in the form of an LED array, an optical diffuser or collimator 1811 (optional), a detector 1805 in the form of a camera, a microprocessor (not shown), and a filter cube 1813. In other embodiments, polarisers may be employed instead of the filter cube 1813.

In this embodiment, where the detector operates in fluorescence mode, the light from the illumination source 1807 may be arranged to pass through an optical diffuser or collimator 1811, an excitation filter 1817 and a dichroic mirror 1819 placed at 45°, in sequence, before reaching the test sample 1803. The emitted light then passes through the dichroic mirror 1819 and the emission filter 1821 that are configured to allow only specific light wavelengths to reach the detector 1805 in the form of a CMOS camera which is connected to a microprocessor (not shown).

In an embodiment, the light shielding cassette 1809 may be made of coloured poly lactic acid (PLA; commercially obtainable) and may be produced via 3D printing.

Advantageously, detectors produced as described above are both light and compact.

Although the specific embodiment shown in FIG. 21(a) is configured to operate in fluorescence mode, in embodiments it can operate in both fluorescent and colourimetry mode according to the camera employed, as will be appreciated by person skilled in the art. For example, a monochrome camera allows only fluorescent mode, whereas use of an RGB camera allows both fluorescent and colourimetry modes.

Thus, this setup also enables the detection of bright field images for colour detection. In such embodiments, a white light source is used, and the light reflected from the sample (coloured light based on colour of the sample) is detected.

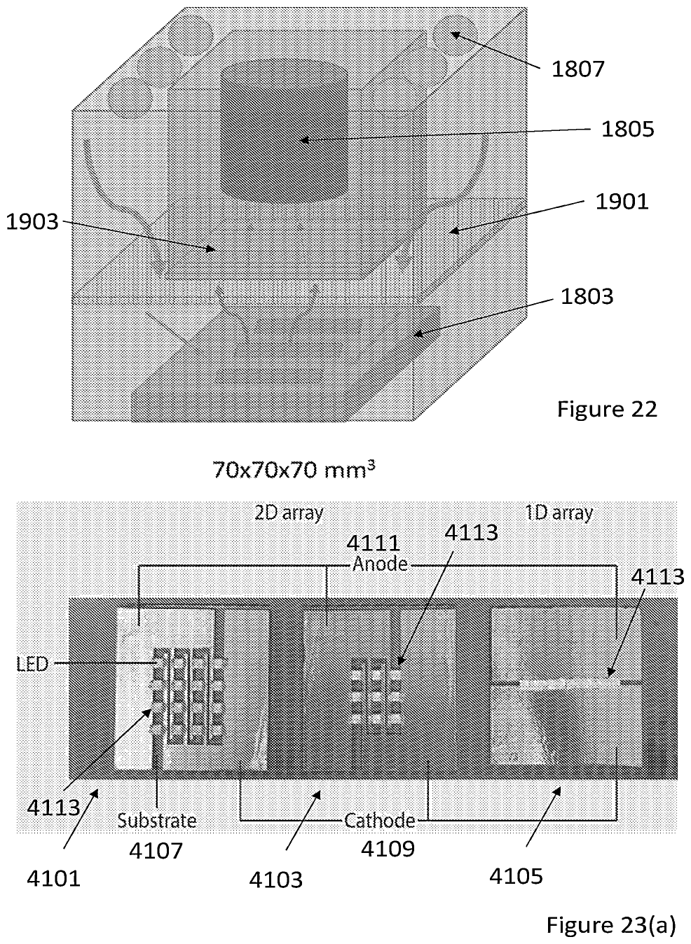

FIG. 22 shows a detector according to another embodiment which is configured to operate in fluorescence mode. In this embodiment, the excitation filters employed in the embodiment of FIG. 21(a) are rendered unnecessary by the use of LEDs for a specific wavelength of light according to the target fluorophore. The emission filters are also unnecessary since the irrelevant wavelengths from the light source will be completely filtered by polarising films placed at 90° to each other. In the embodiment of FIG. 22, two polarizing films 1901, 1903 orientated at 90° to each other are employed. This advantageously enhances the sensitivity of the setup: one polarizing film 1901 is positioned so as to polarize the excitation light from the illumination source 1807 and the other 1903 is positioned before the camera or photodiode/array of photodiodes to block all the excitation light and thereby improve the signal to noise ratio of the detected signal.

In embodiments, the entire optical set-up may or may not have an optional lens which focuses the light onto the camera. Regardless of the precise nature of the optical set up, however, as in the embodiment, of FIG. 21(a) the components are configured such that the field of view of the camera is large enough to capture a plurality of microfluidic channels. In an embodiment, the field of view of the camera is at least about 40×40 mm² and the spatial resolution is at least about 15 μm, particularly about 10 μm or more.

In an embodiment, the components of the detector are modular, enabling adaptation according to the required use. For example, an industrial CMOS camera (grey scale camera) may be employed in embodiments, while an RGB CMOS camera may be employed according to other embodiments. Due to the modularity of the design, the cameras can be directly mounted and onto the detection module and swapped without significant changes other modules in the disclosed system.

FIG. 21(b)-(f) show detectors according to embodiments capable of multiplexing. The detectors are configured to have broadly the same optical arrangement as the embodiment of FIG. 21(a), the light originating from illumination sources 1807, 4629 4631 passing through an excitation filter and a dichroic mirror placed at about 45°, in sequence, before reaching the test sample 1803. The emitted light then passes through the at least one dichroic mirror and an emission filter but enable the illumination of the test sample 1803 using different excitation wavelengths.

Note that in the embodiments of FIGS. 21(a)-(f), the excitation and emission filters are optional, except for FIG. 21(c) where excitation filters are required.

In the embodiment of FIG. 21(b), a white light or light with desired excitation wavelengths as illumination source 1807 passes through an (optional) broadband or multiband excitation filter 4601 and is directed by total reflection mirror 4602 to illuminate and excite the sample 1803; the emission wavelengths are directed to separate cameras 4607, 4609 via respective (optional) separate emission filters 4611, 4613 with respective dichroic mirrors 4603, 4605.

In the embodiment of FIG. 21(c), only a beam splitter 4625 is employed to direct the excitation wavelengths to the sample 1803. A wheel of excitation filters 4614 (shown schematically in FIG. 21(d)) is installed near the illumination source 1807. By rotating the wheel 4614, the excitation filters 4611 and 4613 can be interchanged to illuminate the sample 1803 with different wavelengths. The emission wavelengths can then be captured by the same camera/detector 4607. Note that in this embodiment, the emission filters 4615, 4617 (which may also be installed on a wheel, to enable interchange as appropriate) are optional.

In the embodiment of FIG. 21(e), the detector comprises a single dichroic mirror 4605 (as well as optional excitation 4615 and emission 4611 filters) but these are contained within a cartridge 4619, which can be swapped for a different optical cartridge 4621 containing a dichroic mirror 4603 (as well as optional excitation 4617 and emission 4615 filters). Thus, multiplexing can be achieved by swapping the optical cartridges 4619 and 4621 with corresponding dichroic mirror and/or excitation/emission filters as required.

In the embodiment of FIG. 21(f), light from multiple light sources 4629, 4631 is directed to a beam splitter 4625 which then directs light to the sample 1803. Light from the first light source 4629 is directed using a mirror 4627 which may be a total reflection mirror or a dichroic mirror. Subsequent light sources 4631 are directed with dichroic mirrors 4605. Respective light sources 4629, 4631 can be turned on and off in order to illuminate the sample with different wavelengths according to requirements. In an embodiment, the light sources 4629, 4631 and dichroic mirrors can be replaced with a conventional multi-channel light source.

In an embodiment, LEDs acting as the light source for the camera in the detectors according to embodiments described above can be arranged in a one-dimensional or two-dimensional array. In an embodiment, the LED array may be placed on a substrate and the LED connected in parallel with two electrodes.

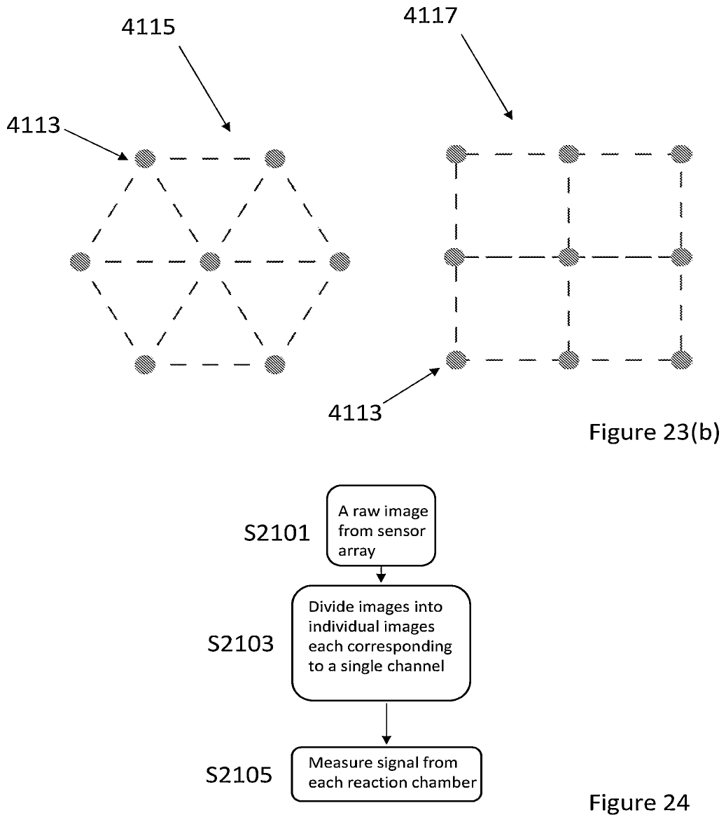

FIG. 23(a) shows three examples of illumination sources 4101, 4103 and 4105 suitable for use in detection modules according to embodiments. 4101 comprises a 4×4 grid array of 16 LEDs 4113. 4103 comprises a 3×3 grid array of 9 LEDs 4113, and 4015 comprises 11 LEDs 4113 arranged in a line. The LEDs 4113 are each connected to cathode 4109 and anode 4111.

FIG. 23(b) shows two further suitable arrangements of LEDs 4113: a triangular array 4115 and a square array 4117. Although the overall arrangement 4115 has an overall hexagonal form, any overall shape in which the LEDs are arranged into sub-units having a triangular arrangement may advantageously provide a uniform intensity distribution.

Likewise, square arrays of LEDs such at 4117 may adopt any overall shape consisting of sub-units having a square arrangement of LEDs according to embodiments.

In an embodiment, the LED array can also be replaced by a single large LED. In this embodiment, with collimation, the LED beams can uniformly illuminate the samples.

In an embodiment, the illumination source comprises an array or arrays of LEDs connected in parallel. An arbitrary array arrangement of an arbitrary number of LEDs can be created using two electrodes 4111, 4109.

In particular, an arrangement of LEDs in which the distance between each LED is substantially identical is employed as it may provide more uniform illumination. Advantageously, triangular arrays may ensure the distances among the closest LEDs are identical. Advantageously, square arrays may enable straightforward manufacture.

The shapes of the cathode 4109 and anode 4111 can be either rectangular, as in illumination source 4105 (for a one-dimensional LED array) or comb-shape as in illumination sources 4101 and 4013 (for a two-dimensional array). Depending on the arrangement of the LED array, the two-dimensional electrodes may take any shape provided they are not connected.

In an embodiment, the substrate for the LEDs may be formed of polymethyl methacrylate (PMMA) and the two-dimensional electrodes 4109, 4111 may be formed from aluminium tape. In other embodiments, the substrate may be glass, plastic or any metallic substrate or a printed circuit board. One-dimensional and two-dimensional arrays made with PMMA, aluminium tape, and LEDs. LEDs may be glued in place with silver epoxy. In this embodiment, the electrical conductivity of silver epoxy may connect the LEDs to the electrodes.

In particular, the substrate is formed of PMMA which may enable straightforward manufacturing. In particular, the substrate is produced using laser cutting.

Detection modules according to embodiments described above provide compact, portable and robust detection modality suitable for a wide range of applications, including but not limited to fluorescence, colourimetry, opacity, precipitation, droplet/bubble generations, as well as direct parasite detection without any amplification such as antigen or antibody test kits. They are able to differentiate signals in spatial domain with high resolution.

Detection modules according to embodiments can be easily integrated with any microfluidic platform that requires image-based detection and analysis. The stationary camera and optical set up of the device provide a high degree tolerance when the sample is not stationary. Furthermore, the wide field of view allows the signal detection from a larger area as opposed the conventional technique of point scanning that focuses on a specific point. Taken together, the embodiments described above offer a simple, cost-effective, compact and portable detection platform for point-of-care diagnostic in both clinical and household setups. Detectors according to these embodiments are also robust and can be easily modified as well as being compact and having low power consumption. They can also be battery operated.

Image Processing Daemon and Pipeline

In an embodiment, a detector according to one of the embodiments of FIGS. 21(a)-(f) and FIG. 22 described above is employed with an image processing daemon that converts images taken by the sensor array in the detection module into signals for analysis or eventual diagnostic.

A general method performed by the daemon is shown in FIG. 24.

In step S2101 the image processing pipeline takes a raw image that is taken from the sensor array as input. The raw image can be a multichannel image, such as RGB image, or a single channel image such as a greyscale image.

In step S2103, the image is divided into a plurality of images, such that each image contains data from only a single reaction channel.

In step S2105, the signal from each of the plurality of divided images is analysed and parameters of interest obtained.

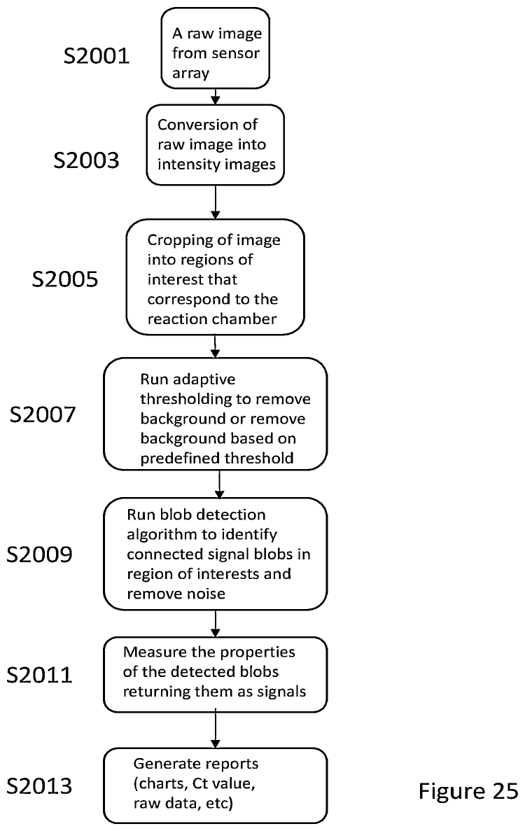

FIG. 25 shows one detailed example of a method performed by the image processing system (daemon) according to the embodiment of FIG. 24.

In step S2001 the image processing pipeline takes a raw image that is taken from the sensor array as input. The raw image can be a multichannel image, such as RGB image, or a single channel image such as a greyscale image.

In step S2003 the image is then converted into an intensity image. In an embodiment, this is done by taking the L2 norm of the image pixels, however the skilled person will appreciate that other methods may be employed according to embodiments.

In step S2005, it is cropped into regions of interest (ROI) that correspond to the reaction channel/chamber. In one embodiment, the image is cropped into 4 regions of interest that correspond to 4 channels on the chip.

In embodiments, there are two possible ways of performing this step:
1) The regions into which the image will be cropped are defined by user prior to running the pipeline using the top left and bottom right position of each ROI; and/or
2) Auto detection of the region of interest using the following steps:
   i. Identify the bright pixels cluster (connected elements) that is at the most top left position of the image. This position is used as the first anchor point of the first channel;
   ii. Based on the identified first anchor point, identify the bottom right most bright pixels cluster for the channel of the first channel. This acts as the second anchor point;
   iii. The ROI of the first channel is obtained by adding a predefined tolerance to the two anchor points and thus identifying the ROI of the first channel.
   iv. Subsequent channels are obtained by offsetting the ROI positions of the first channel by a predefined offset which is determined using the known dimensions and positions of the microfluidic chip and channels.

In step S2007, for each ROI, background noise is removed by intensity thresholding. The intensity threshold can be pre-set or determined by using an adaptive thresholding algorithm, such as but not limited to Otsu's method.

In step S2009 blob detection in each ROI is achieved by detecting connected pixels. In an embodiment, the degree of connectivity is pre-set in system configuration. Noise is further removed by removing blobs with area that is smaller than a pre-set area threshold.

In an embodiment, connected pixels are identified by progressively scanning for bright pixel neighbours of other bright pixels, according to the pre-set degree of connectivity.

For example, if the degree of connectivity in the system configuration is degree 2, a bright pixel is considered connected to another bright pixel if the second bright pixel is a 1 or 2 hop-neighbour to it (i.e. The position of the first pixel is related to the position of the second pixel by offsetting the x or y position at most once in each direction). In other embodiments, degree 1 (i.e. a single hop) connectivity may be employed.

In general, degree 2 connectivity will produce a smoother but larger blob while degree 1 produce more edgy but smaller blob. Both are sufficiently good approximations.

In step S2011, properties of the blobs, which may include but not be limited to intensity histogram, sum of intensity, mean intensity, area, centroid position, within the ROI are measured, recorded and aggregated as signals for downstream analysis.

In step S2013, using these signals, amplification plots and the threshold cycle value (Ct value) are generated by the pipeline as a report.

The person skilled in the art will appreciate that additional or fewer steps may be performed according to embodiments, according to the needs of the user and intended application of the system.

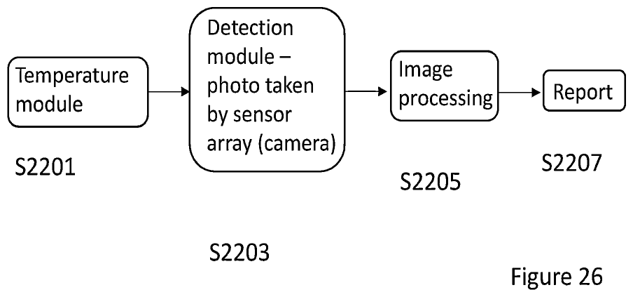

In an embodiment, the daemon is deployed together with the detection module and one or more microfluidic chips according to embodiments described above. This is illustrated according to an embodiment in FIG. 26, in which a method of performing the whole process of detection and diagnosis is shown schematically. This process will now be described in accordance with an embodiment.

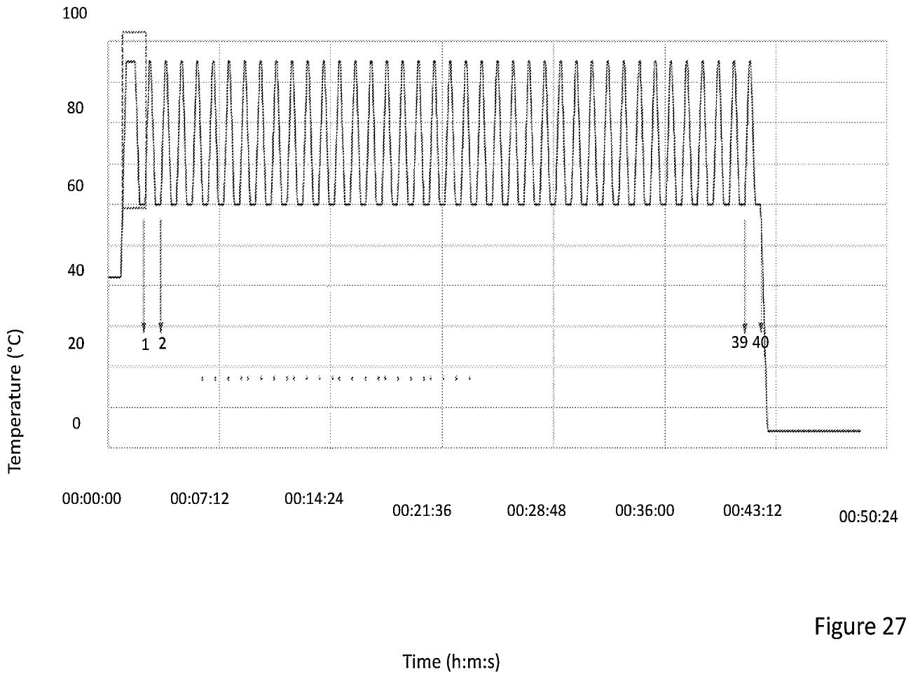

In step S2201 the temperature of the microfluidic chip (temperature module) is controlled as described according to one of the embodiments discussed above. The operation of the microfluidic chip can be adapted according to the requirements of the process. One example is shown in FIG. 27 in which reaction cycles are modulated by the microfluidic chip by oscillating the temperature between two set points, in this case 60° C. and 95° C. over a period of time, indicated by the y-axis.

In step S2203 the sensor array of the detection module takes at least one image of the microfluidic chip. In an embodiment, the detection module may be configured to take multiple images of the microfluidic chip. In an embodiment, the timing of the images may be dependent on the temperature control of the microfluidic chip. For example, the timing of image capture according to one example is indicated by arrows in FIG. 27 (the numbers indicate the count of images captured). In this example, an image is captured each time the temperature reaches its lower setpoint, 60° C. Thus, the sensor array is configured to make a measurement in conjunction to the end of a reaction cycle, as modulated by the temperature module.

In step S2205 the image is processed.

In this embodiment, the image processing daemon may run as a background process and check for new files output from the sensor array.

As new images are captured, they are detected by the daemon. It is then run through the image processing procedure as described in accordance with FIG. 24 or 25.

In Step S2207 a report may be issued with the results obtained from the reaction chamber. The nature of this report will be discussed below.

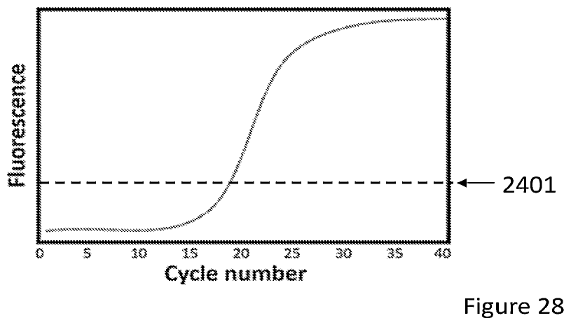

In an embodiment, metrics of the system, such as Ct value (threshold cycle value), and amplification plots for the reactions are updated near real-time. The determination of Ct value according to an embodiment is schematically illustrated in FIG. 28 in which a fluorescence signal exceeds a pre-set threshold value 2401, or Ct, after a given number of cycles (in this example, 20). Ct depends on a variety of factors such as platform, nature of the process being performed and protocol. For example, Ct may be set to a value above which the sample is considered to have tested positive for a particular component.

The ability to process and obtain these metrics near real-time may be important in the application of point-of-care diagnostic as it may hasten the time from sample to results. For example, where an amplitude above Ct indicates that the sample is positive for the detection of viral RNA, and it is determined that a sample has attained the Ct after 20 cycles, then there may be no need to wait until 40 reaction cycles have been run on the sample in order to obtain a measurement. Use of the image processing pipeline to determine Ct in near real-time may therefore greatly enhance detection efficiency.

In an embodiment the daemon may be configured to perform end-point analysis. End-point analysis is employed to determine whether or not an amplification reaction for a particular target is occurring, which, in turn leads to confirmation of the presence or absence of the target. This technique may be employed in screening in which the dynamics of the amplification are not of interest. In end-point analysis mode, at least one image of the microfluidic chip, with samples and reagents loaded, is obtained before any reaction cycles and compared with at least one image of the microfluidic chip at the end of a predetermined number of reaction cycles. The signal for each channel on the chip for the respective images, before and after the reaction cycles, can be quantified using the daemon using the image analysis algorithm as described above according to an embodiment. The difference between the signals (average signals if more than one set of before and after images are employed) before and after the reactions is quantified for each channel respectively. If the difference is statistically significant for a particular channel, the system would report that the target for amplification is present in the sample in that channel. If the difference is statistically insignificant for a particular channel, the system would report that target for amplification is absent in the sample in that channel.

For additional verification, a negative control channel can be used in the microfluidic chip. The difference between the signals received form a channel before and after a given number of reaction cycles is compared to that of the negative control channel. The target is determined to be present if there is statistically significant difference between the two difference signals (before and after the reaction cycles) of the sample channel relative to that of the negative control channel. The target would otherwise be determined to be absent.

For example, for detection of SARS-CoV-2 in a clinical sample, images of the microfluidic chip may be taken before any reaction cycles (i.e. cycle 0), and after 40 reaction cycles according to one protocol (i.e. cycle 40). The difference between the signal of cycle 40 and that of cycle 0 (such as total intensity, or mean intensity) can be used to conclude if the clinical sample is positive to SARS-CoV-2 when the difference is statistically significant while negative to SARS-CoV-2 when the difference is statistically insignificant. In such a case, according to an embodiment, the daemon would report that the person from which the clinical sample is collected from is positive or negative to SARS-CoV-2.

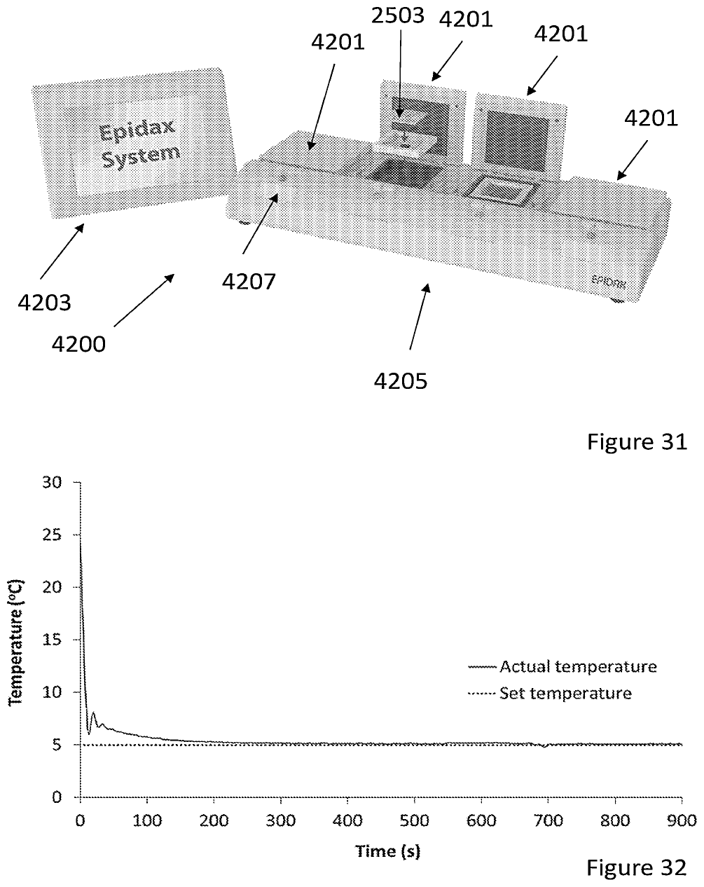

In an embodiment, multiple thermal units could be run in parallel (see, for example, the system of FIG. 31 in which each thermal unit carries one of the microfluidic chips according to embodiments) to further increase the throughput of the system. At the end of the reaction cycles, the microfluidic chips from multiple thermal units could be detected by either one detection module, or multiple detection modules with different wavelengths (for example FAM, HEX, etc.) when there is a need for multiplexed detection.

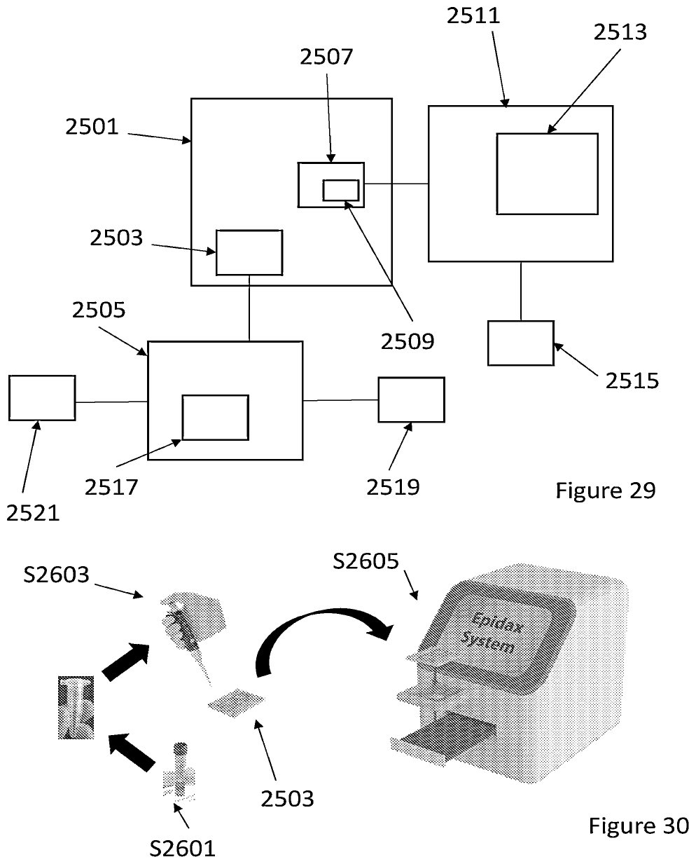

FIG. 29 shows a schematic diagram of a system which performs the method of FIG. 26, combining both one of the microfluidic chips and a detection module according to an embodiment described above. In an embodiment, the system performs amplification and detection of a nucleic acid. In other embodiments, other processes may be performed. Such systems according to embodiments generally comprise a temperature controller, a detection module over the temperature module, power supply and micro-controller, data logger (laptop computer, tablet PC, remote server, etc.)

The system according to an embodiment of FIG. 29 comprises a detection module 2501 in which is placed a microfluidic chip 2503 which is an example of one of the microfluidic chips i.e. in accordance with one of the embodiments described above. The microfluidic chip 2503 may be controlled by an external temperature control device 2505. The temperature control device may comprise a processor 2517 configured to control the temperature of the microfluidic chip 2503. The temperature control device may comprise an input 2521 by which instructions for controlling the temperature of the microfluidic chip 2503 may be received. The input 2521 may include a keyboard, a disk, a connection to a network over which instructions may be received, or any other method of inputting instructions or configuration parameters to the system. The temperature control device may further comprise an external power supply 2519 as discussed above in relation to FIGS. 4 and 5.

The detector module further comprises a camera or imaging system 2507 comprising processor 2509 which outputs data (i.e. serves as an input to) the image processing system 2511. The image processing system comprises a processor 2513 configured to perform an image processing method according to an embodiment. In an embodiment, the processor performs the method of FIG. 24 or 25.

The image processing system 2511 further comprises an output 2515 by which it outputs data. Output devices 2515 may include a video monitor, an output to a disk or network or any other method of outputting data from the system.

Systems according to the embodiment of FIG. 29 may be compact and can be deployable in a point-of-care setting.

Samples and reagents are loaded onto the microfluidic chip 2503 and placed onto the temperature module for processing based on required protocols such as reverse transcription, nucleic acid amplification, isothermal amplification, thermal cycling, etc.

The detection module can be operated independently if continuous measurement is not required by the protocols to be run on the system. In this case, measurement can be taken at required time point or processing steps by placing the detection module 2501 on top of the microfluidic chip 2503.

In an embodiment, the system of FIG. 29 is a portable micro-PCR system as illustrated in FIG. 30. In this embodiment, the sample may be first collected in step S2601 and pipetted into the microfluidic chip 2503 in step S2603 under a biosafety cabinet, placed on a cartridge before being inserted into the micro-PCR system in step S2605 which houses the PCR and detection modules and temperature controller. The microfluidic chip 2503 enclosed within the cartridge may ensure that user will not be exposed to the clinical sample as the chip is being loaded into the machine. Both microfluidic chip 2503 and cartridge may be disposable.

FIG. 31 shows another system 4200 according to an embodiment comprising multiplexed thermal units 4201 (four in the example of FIG. 31) for multiplexed processing to increase throughput. The multiplexed thermal units 4201 can run independent protocols using the same or different microfluidic chips 2503 depending on requirements. The protocols can be configured using screen 4203 or individual input panels 4207 and started independently. In this embodiment, the detection module 4205 is operated independently of the multiplexed thermal units 4201 and measurement of signals is conducted separately when required, such as at the start and at the end of the operation. Additional multiplexed detection modules according to embodiments can also be mounted on the embodiment of FIG. 31 in order to enable continuous measurement.

Advantages of the embodiments described above will now be described with the aid of non-limiting examples.

### Examples

Temperature Control with Microfluidic Chip

Two prototype microfluidic chips according to the embodiments of FIGS. 6(a) and 7(b) were prepared in order to test the temperature control functions of microfluidic chips according to embodiments.

For the first prototype, configured according to the embodiment of FIG. 6(a), the channel plate 803 was manufactured using a 2 mm thick blank made from polymethyl methacrylate (PMMA) to a dimension of 45 mm×45 mm (L×W). A channel profile according to the design of FIG. 6(c) was milled into the blank using a micro milling machine to a channel depth of 500 μm. An O-ring 809 was inserted into the seal groove 805 and a copper film of thickness 35 μm (commercially obtained from RS Components and cut to a size to match the channel plate) was employed as the thermally conductive film 811 and secured to the channel plate by manual application. The spacer was produced by laser cutting of PMMA. The Peltier device (commercially obtained from TE Technology) was placed within the spacer, backed by a support plate. The support plate was machined by using copper material. The components were secured together using snap lock pins and compression springs through holes drilled into the components at the corners.

For the second prototype, having the design shown in FIG. 7(b), the channel plate 715 was manufactured using a 2.8 mm thick blank made from polymethyl methacrylate (PMMA) to a dimension of 45 mm×45 mm (L×W). Four channel profiles according to the design of FIG. 7(c) were cut into the blank using a CO₂ laser with a channel depth of approximately 700 μm. A temperature sensor slot 721 was cut through using a CO₂ laser. An aluminium film of thickness 36 μm (commercially obtained from Sigma Aldrich and cut to a size to match the channel plate) was employed as the heat transfer sealing layer 723 and secured to the channel plate 715 by manual application. The spacer plate 727 was produced by laser cutting of PMMA. The Peltier device 729 (commercially obtained from TE Technology) was placed within the spacer plate 727, backed by the support plate 733. A temperature sensor was inserted into the temperature sensor slot 721. The support plate 733 was machined by using copper material. The components were secured together using magnetic fastening elements through holes drilled into the components at the corners.

The Peltier device and temperature sensor were electrically connected via electrical wires to a temperature controller to which power was supplied from a power supply.

The results of temperature control testing using the two prototypes are shown in FIGS. 32 to 35, with the first prototype being employed in obtaining the results shown in FIGS. 32, 33 and 35 and the second prototype being employed in obtaining the results of FIG. 34.

The temperature set by the temperature controller for the thermally active element is indicated by a dotted line in all of the FIGS. 32 to 35, while the actual temperature detected by a control sensor (see above) is indicated by a solid line. Water was employed as the sample in the channel in all cases.

As shown in FIG. 32, the first prototype was able to perform isothermal control of the sample at 5° C. and to hold the temperature for 900 s, starting from room temperature of ˜25° C. The ramp time to the set temperature was set at 15 s.

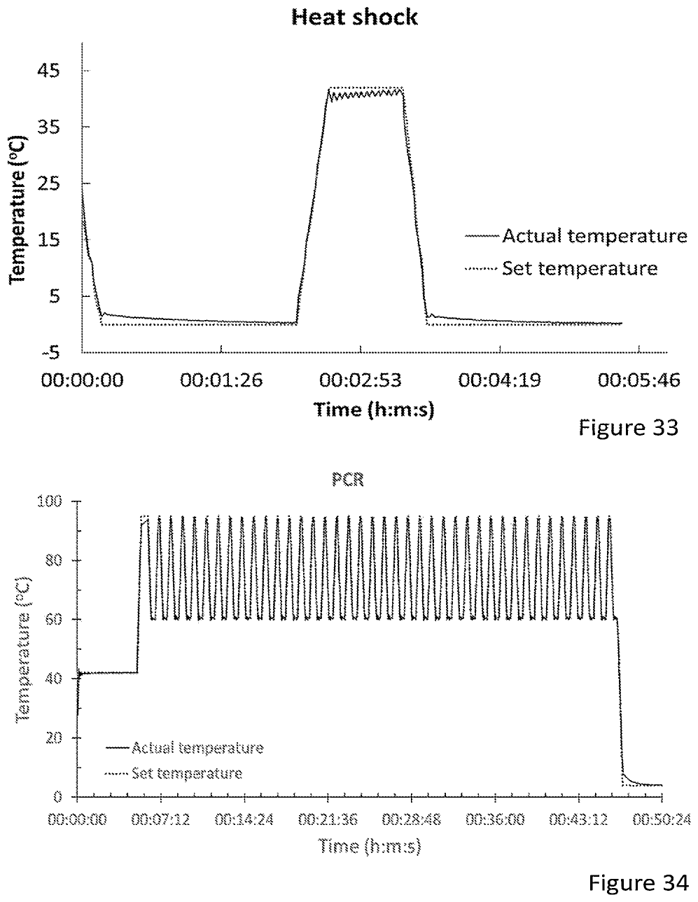

FIG. 33 shows temperature control performed by first prototype in cyclic mode. The results show cyclic temperature control suitable for performing heat shock. The module started from a room temperature of approximately 20° C. and the temperature was reduced to 0° C., holding this temperature for 120 s before increasing to 42° C. and holding 45 s. Cooling to 0° C. and holding for 150 s were subsequently performed. The observed ramp time was 15 s.

FIG. 34 shows temperature control using the second prototype suitable for a reverse transcription PCR cycle, namely heating of the sample up to 42° C. and holding for 5 minutes, 95° C. and holding for 10 s, followed by 40 cycles of 95° C. for 5 s and 60° C. for 20 s, and finally a cool down to 4° C.

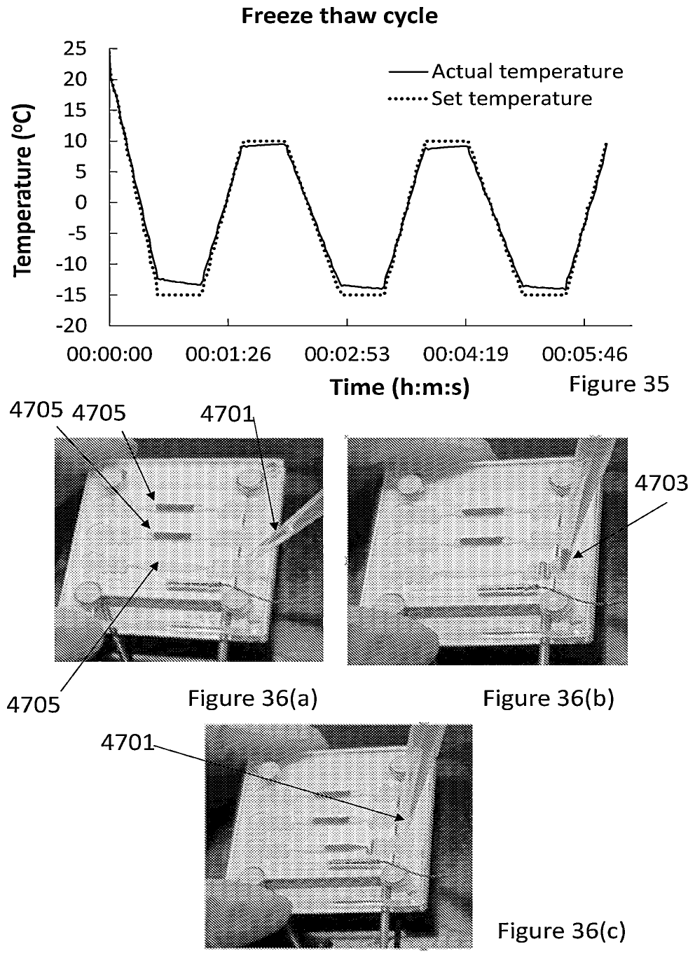

FIG. 35 shows temperature control using the first prototype suitable for a freeze thaw cycle. The protocol comprises 3 cycles of freezing at −15° C. for 30 s and thawing at 10° C. for 30 s.

As can be seen from these figures, the actual temperature detected by the control sensor matches that of the set temperature very closely. Temperature control according to embodiments may therefore be precise and suitable for a range of applications with a small ramp time.

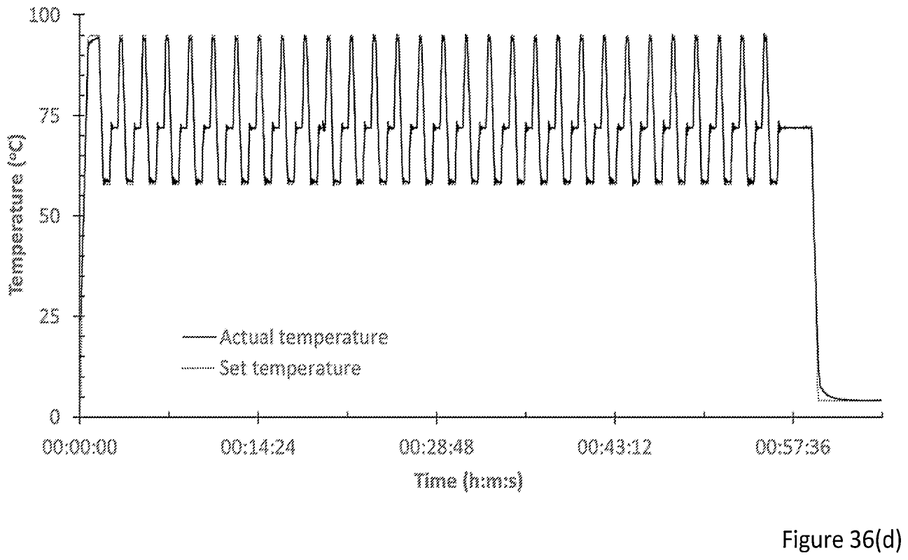

A third prototype microfluidic chip produced in according with the process described above in relation to the second prototype microfluidic chip but with three channels 4705 as opposed to four and a transparent, polyester-based film obtained from Brooks Life Sciences as the heat transfer sealing layer, was employed to evaluate temperature control for a PCR reaction for the evaluation of a gene of interest (in this case a GFP gene) in a DNA plasmid with a size of 2815 base pairs. The third prototype microfluidic chip is shown in FIGS. 36(a) to 36(c). The third prototype microfluidic chip had an overall dimension of 45 mm×45 mm (L×D) and the channel depth was approximately 700 μm. The components were secured together using snap lock pins and compression springs through holes drilled into the components at the corners. The reactions were executed in 10 μL total volume using Q5 High-Fidelity DNA polymerase (New England Biolabs). PCR mix prepared following the manufacturer's manual was mixed with 1 μL of DNA template with a concentration of 430 ng/μL. For better visualization on the chip, an additional 1 μL of DNA loading dye was added into the reaction mix.

The channels were loaded using the oil sandwiching method described above. FIGS. 36(a)-(c) show the steps of the loading process. In FIG. 36(a), 10 μl of mineral oil 4701 was first added to the channels. Subsequently, as shown in FIG. 36(b), the total volume of 11 μL of PCR master mix and dye, primers and DNA plasmid template as described above 4703 was added. Finally, in step FIG. 36(c), another 10 μl of mineral oil 4701 was loaded. The mineral oil was used to prevent evaporation issue during the denaturation step of the PCR cycle.

The PCR reaction was performed on the third prototype microfluidic chip and compared against a tube experiment performed on the Bio-Rad CFX96 (commercially available from Bio-Rad Laboratories, Inc.). Each PCR reaction was performed as follows: heating to 95° C. for 30 s; 30 cycles of 95° C. for 10 s, 58° C. for 30 s, and 72° C. for 30 s; followed by a final extension for 2 min and then cool down to 4° C. as shown in the temperature control profile of FIG. 36(d). All reactions on the third prototype microfluidic and in the tubes were performed in parallel. PCR products from both systems were then evaluated by performing gel electrophoresis and the Bio-Rad ChemiDoc™ Touch Imaging System (commercially available from Bio-Rad Laboratories, Inc.) was used to visualize the results. Gel images of the PCR product using a 1 kb ladder (New England Biolabs) were generated for comparison. DNA bands from the experiments on the third prototype microfluidic chip according to embodiments were comparable to those from the tube experiments on a Bio-Rad CFX96 system (using PCR mixes with loading dye added before the PCR run, to imitate the chip experiment).

The ability to perform deep freezing of sample in microfluidic channel was also tested using a fourth prototype microfluidic chip in accordance with the embodiment illustrated in FIGS. 6(a) and (b).

For this fourth prototype, the channel plate 803 was manufactured using a 2 mm thick blank made from polymethyl methacrylate (PMMA). A single channel (600 μm wide and 500 μm deep) and an O-ring-like groove (1100 μm wide and 750 μm deep) according to the design of FIG. 6(d) were milled into the channel plate. A temperature sensor slot was cut through using a CO₂ laser. A copper film of thickness 35 μm (commercially obtained from RS Components and cut to a size to match the channel plate) was employed as the thermally conductive film and secured to the channel plate by manual application. The spacer was produced by laser cutting of PMMA. The Peltier device (commercially obtained from TE Technology) was placed within the spacer, backed by a support plate. A temperature sensor was inserted into the temperature sensor slot. The support plate was machined by using copper material.

The fourth prototype was tested using water as the sample loaded onto the channel and was able to achieve sub-zero temperature as low as −20° C. within a short period of time. It was visibly observed that the liquid within the channel was frozen and frost formed on the surface of the fourth prototype chip. This proves the potential applicability of embodiments in deep freeze processes, such as freeze-thaw cycle. Microfluidic chips according to embodiments may also be employed for the storing of samples for downstream processes as samples at low temperature can be collected directly from the chip. In the case of droplets microfluidics, water-based droplets are suspended in an oil phase. At temperatures below freezing, such as −20° C., oil remains in liquid phase while the water-based samples freeze, which can be collected into a tube and kept in a freezer.

Channel Designs

The fluidic functions of the main channel illustrated in FIG. 6(d) were tested using a fifth prototype chip produced as described above in relation to the fourth prototype of the microfluidic chip with a single channel and an O-ring-like groove of the design of FIG. 6(d) milled into the channel plate.

Initially, one fluid (coloured yellow) was infused into the single channel, followed by the additional infusion of a second fluid. When both fluids were pumped at high flow, there were low levels of mixing. When flow rates of both fluids were reduced, the two flows mixed better. When the fluids were stopped, the two fluids well mixed by diffusion. This shows the potential to perform DNA transformation on chips according to embodiments. In such processes according to literature protocols (for example the Zymo Research protocol), two fluids may be infused with fluid 1 containing DNA plasmid and fluid 2 containing competent bacterial cells. After the mixing of samples (i.e. DNA plasmid and bacterial cells), there may be a long incubation period (15-20 min or more). Mixing followed by temperature control is therefore necessary.

A sixth prototype microfluidic chip having the channel designs shown in FIGS. 9(c), (e), (b), (a) all present on the single prototype microfluidic chip was produced in order to test the functionality of each design. The sixth prototype microfluidic chip was produced as described above in relation to the second prototype microfluidic chip but with the channel designs created in the plastic blank by laser cutting.

The channel designs were tested by running RT-PCR protocols on samples loaded into the channels.

The RT-PCR protocols such as that of the Takara protocol may include, but are not limited to, raising the temperature above 90° C. (such as 90° C.-95° C.) and holding for a few seconds such as 3-5 s, following by cooling to some lower temperature such as 60° C.-72° C. and holding for some amount of time such as 20-30 s and then repeating the cycle.

The samples used were water with blue dye and they were loaded onto the channels as follows. Each channel was filled with 15 μl of mineral oil; 25 μl of water mixed with blue food dye; and 15 μl of mineral oil. Photos of the microfluidic chip at Cycle 0 (before thermal cycling at room temperature), Cycle 28 and Cycle 44 of thermal cycling were taken, showing that the samples remained in the reaction chamber regions and did not break up into smaller sample units, thus indicating that the designs were functioning.

Likewise, a seventh prototype microfluidic chip with four identical channels on a single chip according to the embodiment of FIG. 7(b), produced in the same way as the second prototype microfluidic chip, but using plastic milling to create the channel design was tested by running the same RT-PCR protocol on samples produced as described above loaded into the channels.

Photos of the seventh prototype microfluidic chip at Cycle 0 (before thermal cycling at room temperature) and Cycle 44 of thermal cycling showed that the sample remained in the reaction chamber regions.

On-Chip Protocols with Temperature Control

An eighth prototype microfluidic chip was produced in accordance with the method described above in relation to the second prototype microfluidic chip but with a channel plate having the design of FIG. 16(b) produced using plastic milling of the blank. Snap lock pins and springs were used as fastening elements to secure components together.

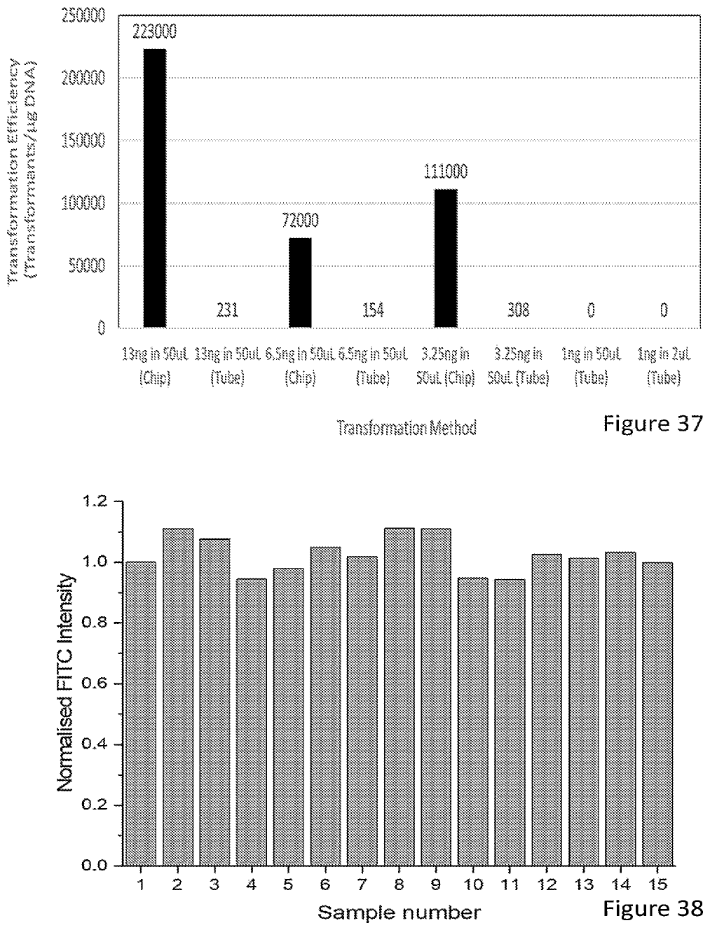

On-chip DNA transformation using this eighth prototype microfluidic chip was demonstrated and compared with conventional transformation in tube by using the following plasmid concentrations (in DNAse free water): 13 ng in 50 μL, 6.5 ng in 50 μL and 3.25 ng in 50 μL. The plasmid used which was GFP nanobody AmpR (from Addgene) was mixed with ice cold 50 μL of competent E. Coli according to vendor protocol (see Zymobroth Fast Transformation of ‘mix-n-go’ competent cells protocol). In the chip, this was achieved by mixing two flows (first flow with plasmid and second flow with competent E. Coli cells), then encapsulating into droplets. The droplets which contained plasmid and competent cells were then collected directly onto an agar plate which selected for successful transformants. In the tube, this was achieved by adding the plasmid directly into the competent cells, followed by gentle mixing. As a control, 2 μL of the plasmid (1 ng in 2 μL) was mixed with 50 μL of the competent cells in the tube. The results for the prototype microfluidic chip were significantly better than that of the tube, with a calculated transformation efficiency of up to 223000 transformants/μg DNA as shown in FIG. 37 for 13 ng in 50 μL for the chip compared with 231 transformants/μg DNA for the tube. It was thus demonstrated that the on-chip transformation employing chips according to embodiments may be efficient.

Platforms according to this embodiment may therefore facilitate the optimization of transformation parameters allowing transformation of a variety of plasmids and cell types inside microfluidic droplet systems.

A ninth prototype microfluidic chip produced as described above in relation to the second prototype microfluidic chip with a channel plate with a design comprising four channels according to the embodiment of FIG. 9(a) produced by laser cutting was employed to perform isothermal amplification as part of reverse transcription loop-mediated isothermal amplification (RT-LAMP).

Two channels on the prototype microfluidic chip were loaded with RT-LAMP reagents, synthetic RNA and primers for the ORF1ab region and N gene respectively; a third channel was a no-template control, containing water, primers and the RT-LAMP reagents only. The fourth channel was left empty.

Primers targeting the ORF1ab and nucleocapsid (N) regions were designed based on Rapid Detection of Novel Coronavirus (COVID-19) by Reverse Transcription Loop-Mediated Isothermal Amplification (Laura E. Lamb et al, medRxiv, 2020), and Rapid Molecular Detection of SARS-CoV-2 (COVID-19) Virus RNA Using Colorimetric LAMP (Yinhua Zhang et al, medRxiv, 2020), respectively, as follows:

ORF1ab Gene (ORF1ab, 5′-3′):
ORF1ab-F3:
(SEQ ID NO: 1)
TCCAGATGAGGATGAAGAAGA
ORF1ab-B3:
(SEQ ID NO: 2)
AGTCTGAACAACTGGTGTAAG
ORF1ab-FIP:
(SEQ ID NO: 3)
AGAGCAGCAGAAGTGGCACAGGTGATTGTGAAGAAGAAGAG
ORF1ab-BIP:
(SEQ ID NO: 4)
TCAACCTGAAGAAGAGCAAGAACTGATTGTCCTCACTGCC
ORF1ab-LF:
(SEQ ID NO: 5)
CTCATATTGAGTTGATGGCTCA
ORF1ab-LB:
(SEQ ID NO: 6)
ACAAACTGTTGGTCAACAAGAC
Nucleocapsid Gene (N, 5′-3′):
GeneN-A-F3:
(SEQ ID NO: 7)
TGGCTACTACCGAAGAGCT
GeneN-A-B3:
(SEQ ID NO: 8)
TGCAGCATTGTTAGCAGGAT
GeneN-A-FIP:
(SEQ ID NO: 9)
TCTGGCCCAGTTCCTAGGTAGTCCAGACGAATTCGTGGTGG
GeneN-A-BIP:
(SEQ ID NO: 10)
AGACGGCATCATATGGGTTGCACGGGTGCCAATGTGATCT
GeneN-A-LF:
(SEQ ID NO: 11)
GGACTGAGATCTTTCATTTTACCGT
GeneN-A-LB:
(SEQ ID NO: 12)
ACTGAGGGAGCCTTGAATACA

A 10× primer mix was prepared according to Zhang et al.: 16 μM of FIP/BIP primers (each), 2 μM of F3 and B3 primers (each), 4 μM of LF and LB primers (each).

RT-LAMP was performed using a set of primers for either Gene N or ORF1ab, and the colorimetric LAMP mastermix (NEB, #M1800L), DNase/RNase free water, and synthetic RNA control (ATCC, #VR-3276SD, diluted to 1000 copies/μL). The reaction mixture contained 2 μL of 10× primer mix, 10 μL LAMP mastermix, 5 μL water and 3 μL of RNA template, and the reaction mixture was set at 65° C. for 30 mins. The temperature module was used to maintain the isothermal condition required by the RT-LAMP protocol.

In general, a detection module is optional in conducting RT-LAMP as presence of target RNA can be determined by the colour change of the sample after RT-LAMP. However, detection modules according to embodiments described above could be employed to perform colorimetry on the resultant sample or fluorescence imaging for fluorescence measurement to obtain quantitative results.

The samples with RT-LAMP reagents were loaded onto the prototype microfluidic chip before RT-LAMP was initiated according to the protocol given above. Samples were pink in colour. The sample changed colour to yellow after RT-LAMP on the chip according to the embodiment. The sample colour was benchmarked to that of RT-LAMP performed in a Bio-Rad CFX96. The colour change achieved on the prototype microfluidic chip was comparable to tube results obtained on the Bio-Rad system.

Tenth and eleventh prototype chips based on the embodiment of FIG. 17(b), employing adhesive sealing film to seal 10 and 12 channels formed in the prototype microfluidic chips, respectively, were produced. The channels 5603 were loaded with water mixed with food dye and heated at 65° C. for 30 minutes in accordance with the RT-LAMP protocol. The channels were examined after the heating and no significant evaporation of the samples was observed.

Detection Module

A first prototype detector according to the embodiment of FIG. 21(a) was built with a filter cube. The dimensions of the detection unit were 110×60×90 mm³ and it weighed approximately 250 g. Thus, detectors according to this embodiment may be light and compact.

The prototype detector was fabricated with a filter cube comprising a dichroic mirror, an excitation filter and emission filter. Due to a circular opening, the field of view was a circle of ϕ40 mm. The filter cube used was Olympus U-MWB2 designed for fluorescein isothiocyanate (FITC) dye.

The prototype detector was fabricated by 3D printing the casing using a commercial 3D printer with designated slots for the LED array, diffuser, filter cube, microfluidic chip and the camera. The material used for the casing was polylactic acid (PLA). A one-dimensional LED array, i.e. illumination source 4105 according to the embodiment shown in FIG. 23(a) was made by employing aluminium tape, as the electrodes and glass as the substrate. Additionally, silver epoxy glue was used to fasten the LEDs to the electrode and a copper wires were connected to the aluminium electrodes to serve as leads to supply the desired voltage or current.

A simple microfluidic chip was fabricated with 2.8 mm polymethyl methacrylate (PMMA) using CO₂ laser cutting. The chip comprised of four identical microfluidic channels with a design according to FIG. 7(c) to hold four samples for detection at a time.

FIG. 38 shows the intensities measured in 15 different channels (obtained by employing the 4-channel chip four times) with values normalised by the first sample of the FITC signal (100 μg/ml) measured by the detection module. The signals obtained with the detection module were consistent. The different channels were infused with 100 μg/ml of FITC solution in DI water. The measurements from the 15 different channels were recorded and the readings were consistent with mean (μ) at 1.023 and standard deviation (σ) at 0.059.

After optimization with FITC dye measurement, the prototype detector was employed to detect signal from PCR results of Ribonuclease P gene plasmid (Hs_RPP30 Positive Control from IDT). For a PCR reaction of 10 μl, 2.5 μl of each different plasmid concentration, specifically 0, 1, 10, 100, 1,000, 10,000, 100,000 and 200,000 copies/μl, was mixed with 7.5 μl of PCR master mix (TaqMan® Fast Virus 1-Step Master Mix from ThermoFisher Scientific) and primers/probe with 5-FAM tag (2019-nCoV CDC EUA Kit from IDT). The mixtures were then used to perform 40 cycles of PCR on Bio-Rad CFX96 qPCR machine (commercially obtained from Bio-Rad Laboratories, Inc). At the end of cycle 40, the mixtures were loaded onto the PCR channel of the prepared chip to be detected with the prototype detector.

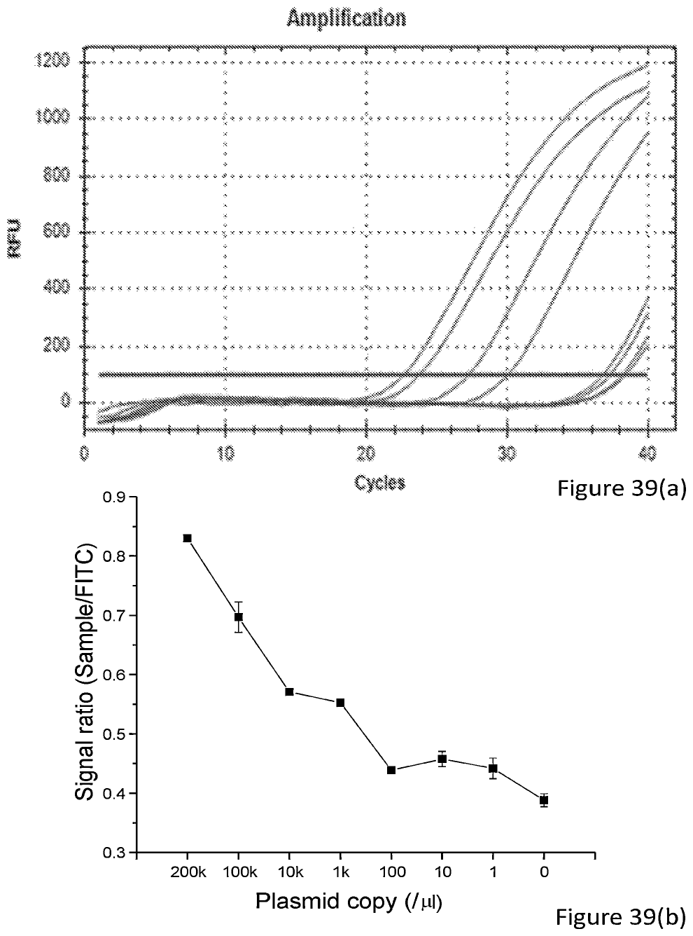

FIG. 39(a) shows the fluorescence signal detected by a Bio-Rad CFX96 qPCR machine at individual PCR cycle for 40 cycles. The curves from left to right corresponding to 200,000, 100,000, 10,000, 1,000, 100, 10, 1, 0 copies/μl respectively.

FIG. 39(b) shows the results using the prototype detector at the end of cycle 40.

As is shown by comparing FIGS. 39(a) and (b), fluorescence signals obtained with the prototype detector were consistent with the readout from Bio-Rad qPCR machine.

A second prototype detector according to an embodiment was fabricated in the same manner as the first.

FIGS. 40(a)-(d) show results for this second prototype detector benchmarked against the commercially available tube-based Bio-Rad CFX96 qPCR machine for sensitivity based on amplification efficiency measured as mean fluorescence intensity. The benchmark experiments were conducted using samples of volume 10 μl, particularly 2.5 μl of synthetic RNA at concentration of 10 copies/μl (AcroMetrix™ Coronavirus 2019 COVID-19 (RNA control (RUO)) was mixed with 7.5 μl of RT-PCR master mix (TaqPath™ 1-Step RT-qPCR Master Mix, ThermoFisher Scientific), primers and probes for N1 gene of SARS-CoV-2 from IDT (product codes IDT 2019-nCoV_N1). 42 PCR cycles were run on the prototype system according to embodiments with a microfluidic chip consisting of 4 channels as shown in FIG. 9(a) with each channel being loaded with 10 μl of sample. Fluorescence images at Cycle 0 (FIG. 40(a)) and Cycle 42 (FIG. 40(b)) on the prototype system are shown. Temperature profile of the 42 PCR cycles is shown in FIG. 40(d). Concurrently, 42 PCR cycles were run on Bio-Rad CFX96 qPCR machine and then the samples were loaded onto an empty microfluidic chip for the detection of signals using the second prototype detector according to an embodiment. The signal strength was measured as the mean fluorescence intensity across each channel as plotted in FIG. 40(c). The mean intensity measured in the four channels at the end of Cycle 42 (FIG. 40(b)) on the second prototype detector according to an embodiment were comparable to that of the Bio-Rad machine (FIG. 40(c)). This indicates that the amplification efficiency may be comparable.

To conduct qPCR using the prototype system, the second prototype detector was used in tandem with the temperature module. Fluorescence images of the channel were taken at each amplification cycle and the images fed into a data logger in real-time while software according to embodiments described above conducted measurements at each cycle.

The Ct value (see discussion above) was also measured using the Bio-Rad machine.

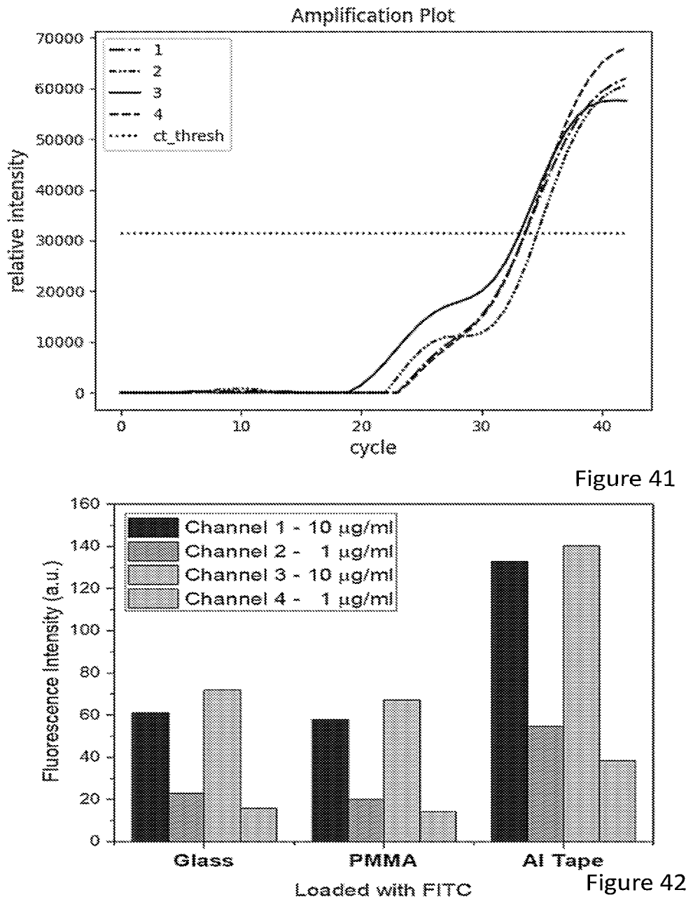

The amplification plot using total intensity obtained using the system described above after smoothing and baseline correction is plotted in FIG. 41. A 1D Gaussian filter was used for smoothing. For baseline subtraction, linear regression over the initial cycles (typically cycles 3 to 15) was used to establish a baseline. The Ct values obtained i.e. the cycle number at which the signal exceeded Ct (ct_thresh), for the 4 channels were 34, 35, 34 and 34 respectively. This is comparable to the Ct value of 35.08 obtained using the tube-based Bio-Rad machine.

To characterize the effect of different composition of microfluidic chips, the detection of FITC dye loaded in microfluidic channels was performed with three kinds of substrate: a glass slide (transparent), a smooth piece of PMMA (transparent), and aluminium film (non-transparent and reflective). A channel plate was manufactured using a 2 mm thick blank made from polymethyl methacrylate (PMMA). Channels according to the design of FIG. 7(c) were laser cut into the plate using CO₂ laser cutting. PMMA glue was used to bond the laser cut chip to the substrate.

As shown in FIG. 42 a two-fold increase in fluorescence signal could be observed from the aluminium film in comparison to the glass and PMMA substrates. This is likely due to the reflection of the fluorescence signal by the aluminium surface, which may serve as a signal amplifier and hence help to increase the sensitivity of the system.

To minimize the background noise and uneven distribution, a third prototype detector according to FIG. 21(a) was fabricated using the same method and according to the same design as the first prototype but with black PLA material.

The third prototype detector was tested with a twelfth prototype microfluidic chip having four PCR reaction channels of the design shown in FIG. 7(c) fabricated as described above in relation to the second prototype microfluidic chip.

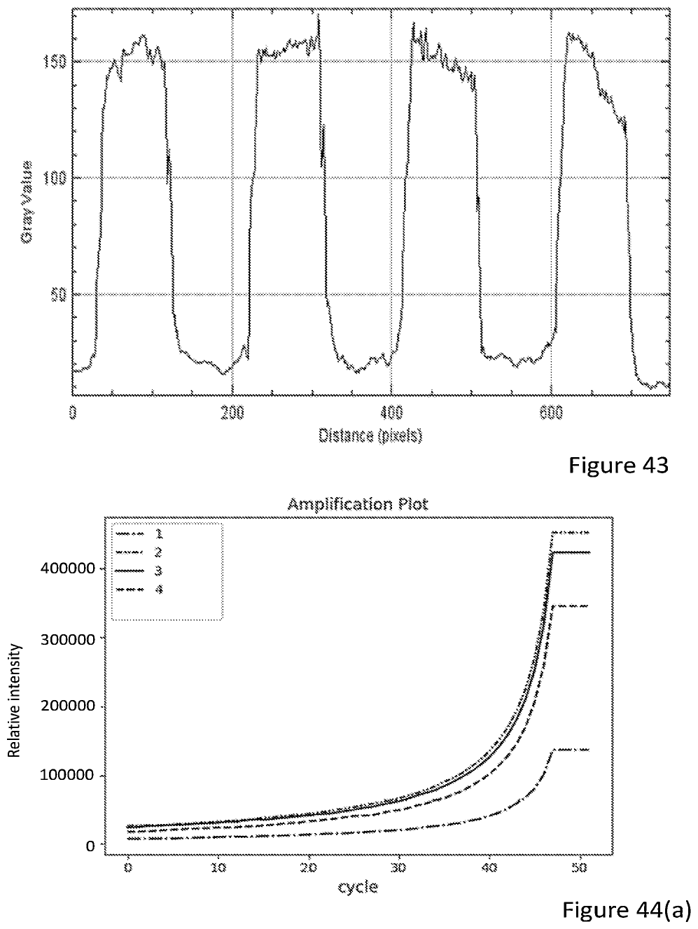

The fluorescence readout recorded was consistent and the background noise was minimal, as shown in FIG. 43 in which is plotted the quantification of fluorescence distribution along a straight line through the four PCR reaction channels, each channel being loaded with 50 μg/ml of FITC in DI water.

This third prototype detector was employed to detect the SARS-CoV-2 synthetic RNA sample (VR-3276SD™ from ATCC) after 40 cycles of PCR—using TaqPath™ 1-Step RT-qPCR master mix from ThermoFisher Scientific and primers for N1 and N2 genes from IDT (product codes IDT 2019-nCoV_N1 and IDT 2019-nCoV_N2 respectively). The synthetic RNA sample was first diluted in nuclease free water to achieve various concentrations as 100,000 copies/μl, 10,000 copies/μl, 1,000 copies/μl, 100 copies/μl, copies/μl and 1 copy/μl. After that, 2.5 μl of RNA sample of each concentration was mixed with 7.5 μl of PCR master mix and primers for a PCR reaction of 10 μl. The mixtures were then used to perform 40 cycles of PCR on Bio-Rad CFX96 qPCR machine.

At the end of cycle 40, the mixtures were loaded onto a PCR channel on the twelfth prototype microfluidic chip described above to be detected with the third prototype detection module. With reduced background signal and uniform excitation, the sensitivity of the detection system was high and fluorescence signals from sample concentration as low as 10 copies/μl were captured.

Daemon

To examine the processing of the images by a daemon according to embodiments described above, proxy images were used to simulate images taken by the sensor array. The proxy images were created as a set of RGB with intensities adjusted to 50 incremental exponential steps corresponding to 50 cycles of PCR reactions in order to simulate the exponential increment of intensity expected in the exponential phase of the PCR reaction. Four reaction channels were simulated.

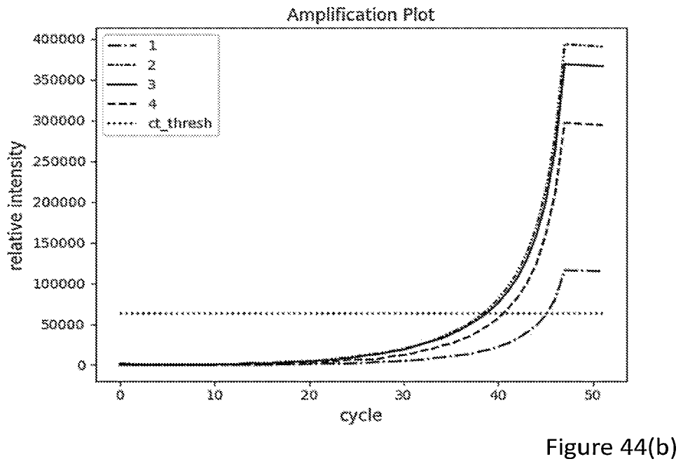

As an image in the proxy image set was created and then detected by the daemon, it was converted into an intensity image and cropped into the ROI. For each ROI, blobs were detected, and signals measured, such as intensity histogram, fluorescence value and Ct value (if applicable). As each subsequent proxy image was created, the daemon updated the amplification plot of FIG. 44(a) (without baseline subtraction) and FIG. 44(b) (with baseline subtraction) in near real-time and the Ct value for each channel was detected as soon as the fluorescence value passed the threshold, which was set to the total intensity of 60000. For baseline subtraction, linear regression over the initial cycles (typically cycles 3 to 15) was used to establish a baseline.

To demonstrate the actual signals that can be obtained from the image processing daemon according to embodiments, an image taken after running through 40 PCR cycles in a four-channel PCR reaction microfluidic chip using the third prototype detector was passed through the daemon. After processing by the daemon, the blobs were detected. The signals computed from the blobs are tabulated in Table 1. Given a set of images taken across reaction cycles, amplification plots and Ct values were obtained by the daemon.

TABLE 1
Signals computed by the daemon

| | Average | Fluorescence | Log |
|---|---|---|---|
| Channel | intensity | value | (Fluorescence value) |
| 0 | 67 | 704037 | 5.85 |
| 1 | 72 | 697858 | 5.84 |
| 2 | 0 | 0 | — |
| 3 | 95 | 1360296 | 6.13 |

The image analysis daemon and pipeline according to embodiments were also employed to perform image analysis on images captured by the second detection prototype described above at various cycles of PCR. The image processing daemon automatically ran the image processing pipeline on new images as they were captured and updated the reports (such as the amplification plots). Fluorescent samples were automatically detected as foreground in each of the four channels of the chip according to an embodiment. The image processing daemon then measured the signals as the mean intensity and the luminous flux over the area of the samples.

Having now described the invention, it should be apparent to one of ordinary skill in the art that many modifications can be made hereto without departing from the scope as claimed.

1. A microfluidic chip comprising:
at least one microfluidic reservoir having a wall portion and a heat transfer sealing layer cooperating with the wall portion for receiving a sample to be tested, the heat transfer sealing layer being arranged to be contiguous with the sample to be tested; and
an active temperature control device arranged to provide structural support to the heat transfer sealing layer and operable to control a temperature of the sample via transmission of heat through the heat transfer sealing layer.

2. A microfluidic chip according to claim 1, wherein the transmission of heat through the heat transfer sealing layer includes transmission of heat from the sample to the active temperature control device.

3. (canceled)

4. A microfluidic chip according to claim 1, wherein the heat transfer sealing layer comprises an adhesive layer or a metallic film, or is integral with the active temperature control device.

5. (canceled)

6. (canceled)

7. A microfluidic chip according to claim 1, wherein the wall portion defines a first microfluidic reservoir profile and wherein the first microfluidic reservoir profile comprises a first periodically oscillating section.

8. A microfluidic chip according to claim 7, wherein the first microfluidic reservoir profile further comprises two or more substantially linear sections and a junction fluidically connecting the two or more substantially linear sections to the first periodically oscillating section.

9. A microfluidic chip according to claim 1, wherein the wall portion defines a second microfluidic reservoir profile and wherein the second microfluidic reservoir profile comprises: a first periodically oscillating section; a second periodically oscillating section; a third section arranged between the first and second periodically oscillating sections, the third section having a non-oscillating configuration and comprising a first chamber region; first and second tapering portions which fluidically connect the first and second periodically oscillating sections, respectively, to the first chamber region; and a width, the width being greater at the first chamber region than in the first and second oscillating sections.

10. A microfluidic chip according to claim 9, wherein each of the first and second tapering portions is curved.

11. A microfluidic chip according to claim 9, wherein each of the first and second tapering portions is substantially straight.

12. A microfluidic chip according to claim 9, wherein the third section further comprises a second chamber region, and wherein the microfluidic reservoir profile further comprises third and fourth tapering portions which fluidically connect the first and second periodically oscillating sections to the second chamber region, respectively, wherein the width is greater at the second chamber region than in the first and second periodically oscillating sections.

13. A microfluidic chip according to claim 1, wherein the wall portion defines a third microfluidic reservoir profile comprising a first periodically oscillating section arranged to oscillate in a first direction; a second periodically oscillating section arranged to oscillate in the first direction; and a third periodically oscillating section positioned between and fluidically connected to the first and second periodically oscillating sections and arranged to oscillate in a second direction which is perpendicular to the first direction.

14. A microfluidic chip according to claim 1, wherein the wall portion defines a fourth microfluidic reservoir profile and wherein the microfluidic chip further comprises an elastomeric seal arranged to surround the fourth microfluidic reservoir profile and wherein the heat transfer sealing layer cooperates with the wall portion via the elastomeric seal.

15. (canceled)

16. (canceled)

17. A microfluidic chip according to claim 1, wherein the at least one microfluidic reservoir comprises at least one of a second inlet and a second outlet, the microfluidic chip further comprising an adhesive sealing layer arranged to seal the at least one of the second inlet and the second outlet.

18. A microfluidic chip according to claim 1 further including a reservoir plate comprising: the wall portion and an edge surface, and wherein the at least one microfluidic reservoir comprises a third inlet comprising an aperture in the edge surface of the reservoir plate.

19. A microfluidic chip according to claim 1, comprising:
a plurality of the microfluidic reservoirs, each microfluidic reservoir having a respective wall portion, wherein the heat transfer sealing layer cooperates with each of the respective wall portions for receiving a respective sample to be tested, the heat transfer sealing layer being arranged to be contiguous with each respective sample to be tested, the active temperature control device operable to control a temperature of each of the respective samples via transmission of heat through the heat transfer sealing layer.

20. A microfluidic chip according to claim 19, wherein each respective wall portion comprises a through hole.

21. A microfluidic chip according to claim 19, wherein each respective wall portion defines a respective microfluidic reservoir profile, and wherein at least two of the respective reservoir profiles differ in at least one of shape and sample capacity.

22. A microfluidic chip comprising:
at least one microfluidic reservoir having a wall portion and a heat transfer sealing layer cooperating with the wall portion for receiving a sample to be tested, the heat transfer sealing layer being arranged to be contiguous with the sample to be tested; and
a fluidic heat exchanger operable to control a temperature of the sample via transmission of heat through the heat transfer sealing layer.

23. A microfluidic chip according to claim 22, wherein the heat transfer sealing layer includes a metal plate.

24. A microfluidic system comprising a microfluidic chip according to claim 1; and a further at least one microfluidic reservoir comprising a further wall portion, and a further heat transfer sealing layer cooperating with the further wall portion for receiving a further sample to be tested, the further heat transfer sealing layer being arranged to be contiguous with the further sample to be tested, the further at least one microfluidic reservoir operable to replace the at least one microfluidic reservoir, the active temperature control device being further operable, upon replacement of the at least one microfluidic reservoir with the further at least one microfluidic reservoir, to provide structural support to the further heat transfer sealing layer and to control a temperature of the further sample via transmission of heat through the further heat transfer sealing layer.

25. A microfluidic platform comprising a plurality of fluidically connected microfluidic chips, the plurality of microfluidic chips including one or more microfluidic chips according to claim 1.

26-45. (canceled)
